(19) United States
(12) Patent Application Publication
(10) Pub. No.: US 2024/0358758 A1
(43) Pub. Date: Oct. 31, 2024

(54) METHODS FOR TREATMENT OF AUTOIMMUNE DISEASES

(71) Applicant: Nkarta, Inc., South San Francisco, CA (US)

(72) Inventors: David Richard Shook, Pacifica, CA (US); James Barnaby Trager, Albany, CA (US); Nishi Kothari, Santa Monica, CA (US); Alison Blaus, Morristown, NJ (US); Phung Gip, San Francisco, CA (US); Alexandra Leida Liana Lazetic, San Jose, CA (US); Sandeep Hora, Dublin, CA (US); Wendy Yu, San Mateo, CA (US); Meriam Vejiga, Foster City, CA (US); Mira Tohmé, San Francisco, CA (US)

(21) Appl. No.: 18/626,710

(22) Filed: Apr. 4, 2024

Related U.S. Application Data
(60) Provisional application No. 63/494,902, filed on Apr. 7, 2023.
(60) Provisional application No. 63/523,598, filed on Jun. 27, 2023.
(60) Provisional application No. 63/544,111, filed on Oct. 13, 2023.
(60) Provisional application No. 63/618,144, filed on Jan. 5, 2024.
(60) Provisional application No. 63/567,812, filed on Mar. 20, 2024.

(51) Int. Cl. A61K 35/17; A61K 31/675; A61K 39/00; C12N 5/0783

(52) U.S. Cl. CPC A61K 35/17; A61K 31/675; A61K 39/4613; A61K 39/4631; A61K 39/464412; C12N 5/0646; A61K 2239/13; A61K 2239/17; A61K 2239/21; A61K 2239/38

(57) ABSTRACT

Provided herein are methods of treating subjects having or suspected of having an autoimmune disease (e.g., systemic lupus erythematosus and/or lupus nephritis) with natural killer (NK) cells, and related compositions, uses, and articles of manufacture. In some aspects, the NK cells express a recombinant receptor, such as a CD19-directed chimeric antigen receptor (CAR).

### CROSS-REFERENCE TO RELATED APPLICATIONS

This application claims priority to U.S. Provisional Application No. 63/494,902, filed Apr. 7, 2023, U.S. Provisional Application No. 63/523,598, filed Jun. 27, 2023, U.S. Provisional Application No. 63/544,111, filed Oct. 13, 2023, U.S. Provisional Application No. 63/618,144, filed Jan. 5, 2024, and U.S. Provisional Application No. 63/567,812, filed Mar. 20, 2024, the entire contents of each of which is incorporated by reference herein.

### FIELD

The present disclosure relates in some aspects to methods of treating autoimmune diseases (e.g., lupus) with natural killer (NK) cells, and related compositions, uses, and articles of manufacture. The NK cells generally express recombinant receptors, such as chimeric antigen receptors (CARs) for targeting an antigen, such as CD19. In some embodiments, the subject has or is suspected of having an autoimmune disease (e.g., a B cell-mediated autoimmune disease). In some embodiments, the subject has or is suspected of having systemic lupus erythematosus (SLE) and/or lupus nephritis (LN).

### BACKGROUND

Autoimmune diseases include a myriad of heterogenous conditions in which a subject's immune system attacks the subject's healthy cells, tissues, and/or organs. B cells contribute to autoimmune disease pathogenesis in multiple ways, including by producing autoantibodies, serving as antigen-presenting cells (APCs), and producing cytokines. Current strategies for treating autoimmune diseases include use of corticosteroids, immunosuppressive agents, and B cell-targeting agents. Such strategies often have limited potency and/or persistence, are not suitable for chronic use, or both. Thus, effective therapies for patients with autoimmune diseases are needed. Provided are methods and uses that meet such needs.

### INCORPORATION BY REFERENCE OF MATERIAL IN SEQUENCE LISTING FILE

This application incorporates by reference the material contained in the Sequence Listing XML file being submitted concurrently herewith: File name: NKT104A_ST26.xml; created on Apr. 2, 2024 and is 39,468 bytes in size.

### SUMMARY

Provided herein are methods of treating an autoimmune disease, the methods comprising administering to a subject having an autoimmune disease a composition comprising natural killer (NK) cells genetically engineered to express a chimeric antigen receptor (CAR) that binds to CD19. In some embodiments, the autoimmune disease is a B cell-mediated autoimmune disease.

Also provided herein are methods of reducing B cells in a subject, the methods comprising administering to a subject having a B cell-mediated disease a composition comprising natural killer (NK) cells genetically engineered to express a chimeric antigen receptor (CAR) that binds to CD19. Also provided herein are methods of reducing the level of an autoantibody in a subject having a B cell-mediated disease, the methods comprising administering to a subject having a B cell-mediated disease a composition comprising natural killer (NK) cells genetically engineered to express a chimeric antigen receptor (CAR) that binds to CD19.

Also provided herein is a method of reducing B cells in a subject having a B cell-mediated disease comprising administering to the subject a composition comprising natural killer (NK) cells genetically engineered to express a chimeric antigen receptor (CAR) that binds to CD19, wherein: (i) the composition comprising the NK cells genetically engineered to express a CAR is administered to the subject in a dosing regimen comprising a dosing cycle; and (ii) the method reduces peripheral B cells in the subject by at least about 90%; peripheral B cells are significantly reduced in the subject for the duration of the dosing cycle; and/or at least about 75% of repopulating peripheral B cells are non-class-switched B cells. In some embodiments, the method reduces peripheral B cells in the subject by at least about 90%. In some embodiments, peripheral B cells are significantly reduced in the subject for the duration of the dosing cycle. In some embodiments, at least about 75% of repopulating peripheral B cells are non-class-switched B cells.

In some embodiments, the B cell-mediated disease is an autoimmune disease. In some embodiments, the genetically engineered NK cells are allogeneic to the subject.

In some embodiments, the method reduces B cells in the subject by at least about 70%, at least about 75%, at least about 80%, at least about 85%, at least about 90%, at least about 91%, at least about 92%, at least about 93%, at least about 94%, at least about 95%, or at least about 99%. In some embodiments, the method reduces B cells in the subject by at least about 70%. In some embodiments, the method reduces B cells in the subject by at least about 75%. In some embodiments, the method reduces B cells in the subject by at least about 80%. In some embodiments, the method reduces B cells in the subject by at least about 85%. In some embodiments, the method reduces B cells in the subject by at least about 90%.

In some embodiments, the method reduces B cells in the subject for at least about 30 days, at least about 45 days, at least about 60 days, at least about 75 days, or at least about 90 days. In some embodiments, the method reduces B cells in the subject for at least about 30 days. In some embodiments, the method reduces B cells in the subject for at least about 45 days. In some embodiments, the method reduces B cells in the subject for at least about 60 days. In some embodiments, the method reduces B cells in the subject for at least about 75 days. In some embodiments, the method reduces B cells in the subject for at least about 90 days. In some embodiments, the method reduces B cells in the subject for about 30 days, about 45 days, about 60 days, about 75 days, or about 90 days. In some embodiments, the method reduces B cells in the subject for about 30 days. In some embodiments, the method reduces B cells in the subject for about 45 days. In some embodiments, the method reduces B cells in the subject for about 60 days. In some embodiments, the method reduces B cells in the subject for about 75 days. In some embodiments, the method reduces B cells in the subject for about 90 days. In some embodiments, the B cells are peripheral B cells.

In some embodiments, the autoimmune disease is a T cell-mediated autoimmune disease. In some embodiments, the autoimmune disease is a plasma cell-mediated autoimmune disease.

In some embodiments, the subject is seropositive for an autoantibody. In some embodiments, the autoantibody is associated with the autoimmune disease. In some embodiments, the autoantibody is associated with the B cell-mediated disease. In some embodiments, the autoantibody is an anti-nuclear antibody (ANA). In some embodiments, the autoantibody is an anti-thyroid antibody. In some embodiments, the autoantibody is an anti-neutrophil cytoplasmic antibody (ANCA). In some embodiments, the autoantibody is an anti-thrombin antibody. In some embodiments, the autoantibody is an anti-citrullinated peptide (CP) antibody. In some embodiments, the autoantibody is an anti-actin antibody. In some embodiments, the autoantibody is an anti-phospholipid antibody. In some embodiments, the autoantibody is an anti-smooth muscle antibody. In some embodiments, the autoantibody is an anti-mitochondrial antibody. In some embodiments, the autoantibody is an anti-ganglioside antibody. In some embodiments, the autoantibody is an anti-signal recognition peptide (SRP) antibody. In some embodiments, the autoantibody is an anti-nicotinic acetylcholine receptor (AChR) antibody. In some embodiments, the autoantibody is an anti-muscle-specific kinase (MuSK) antibody. In some embodiments, the autoantibody is an anti-voltage-gated calcium channel (VGCC) antibody. In some embodiments, the autoantibody is an anti-Vinculin antibody. In some embodiments, the autoantibody is an anti-Hu (ANNA-1) antibody. In some embodiments, the autoantibody is an anti-RF antibody. In some embodiments, the method reduces the level of an autoantibody in the subject.

In some embodiments, the subject is seropositive for an anti-EBV antibody.

In some embodiments, the autoimmune disease is selected from the group consisting of systemic lupus erythematosus (SLE), lupus nephritis (LN), scleroderma, rheumatoid arthritis (RA), myasthenia gravis (MG), multiple sclerosis (MS), NMDA/NMDAR encephalitis, transverse myelitis, neuromyelitis optica spectrum disorder (NMOSD), myelin oligodendrocyte glycoprotein antibody disease (MOGAD), myelin oligodendrocyte glycoprotein spectrum disorder (MOGSD), idiopathic inflammatory myopathy (IIM; also known as myositis), Sjogren's disease, pemphigus vulgaris, bullous pemphigoid (BP), membranous nephropathy (MN), immune thrombocytopenia (ITP), Hashimoto' disease, Grave's disease, insulin resistance, type I diabetes, antiphospholipid syndrome, vasculitis, anti-neutrophilic cytoplasmic antibodies (ANCA) vasculitis (AAV), and anti-synthetase syndrome (ASSD). In some embodiments, the autoimmune disease comprises idiopathic inflammatory myopathy (IIM), multiple sclerosis (MS), myasthenia gravis (MG), rheumatoid arthritis (RA), scleroderma, thyroid disease, type 1 diabetes, vasculitis, or any combination thereof. In some embodiments, the autoimmune disease is selected from the group consisting of SLE, LN, scleroderma, MG, IIM, and vasculitis.

In some embodiments, the autoimmune disease comprises scleroderma. In some embodiments, the autoimmune disease is scleroderma. In some embodiments, the autoimmune disease comprises systemic sclerosis (also known as systemic scleroderma). In some embodiments, the autoimmune disease is systemic sclerosis (also known as systemic scleroderma). In some embodiments, the autoimmune disease comprises localized scleroderma. In some embodiments, the autoimmune disease is localized scleroderma.

In some embodiments, the autoimmune disease comprises myositis (also known as IIM). In some embodiments, the autoimmune disease is myositis (also known as IIM). In some embodiments, the autoimmune disease is selected from the group consisting of anti-synthetase syndrome (ASSD), overlap myopathy (OM), dermatomyositis (DM), clinically amyopathic dermatomyositis, juvenile myositis (JM), necrotizing myopathy (NM; e.g., necrotizing autoimmune myopathy (or immune-mediated necrotizing myopathy), polymyositis (PM), and sporadic inclusion body myositis (sIBM). In some embodiments, the autoimmune disease is ASSD. In some embodiments, the autoimmune disease is OM. In some embodiments, the autoimmune disease is DM. In some embodiments, the autoimmune disease is JM. In some embodiments, the autoimmune disease is NM. In some embodiments, the autoimmune disease is PM. In some embodiments, the autoimmune disease is sIBM.

In some embodiments, the autoimmune disease comprises vasculitis. In some embodiments, the autoimmune disease is vasculitis. In some embodiments, the vasculitis is large-vessel vasculitis. In some embodiments, the vasculitis is medium-vessel vasculitis. In some embodiments, the vasculitis is small-vessel vasculitis. In some embodiments, the vasculitis is anti-neutrophilic cytoplasmic autoantibody (ANCA) vasculitis. In some embodiments the ANCA vasculitis is granulomatosis with polyangiitis (GPA). In some embodiments the ANCA vasculitis is microscopic polyangiitis (MPA). In some embodiments the ANCA vasculitis is eosinophilic granulomatosis with polyangiitis (EGPA).

In some embodiments, the autoimmune disease comprises myasthenia gravis (MG). In some embodiments, the autoimmune disease is MG. In some embodiments, MG is ocular MG. In some embodiments, MG is early-onset generalized MG. In some embodiments, MG is late-onset MG.

In some embodiments, the autoimmune disease comprises multiple sclerosis (MS). In some embodiments, the autoimmune disease is MS. In some embodiments, MS is primary progressive MS (PPMS). In some embodiments, MS is secondary-progressive MS (SPMS). In some embodiments, MS is relapsing-remitting MS (RRMS).

In some embodiments, the autoimmune disease comprises systemic lupus erythematosus (SLE). In some embodiments, the autoimmune disease is systemic lupus erythematosus (SLE). In some embodiments, the autoimmune disease is SLE without lupus nephritis (LN).

In some embodiments, the autoimmune disease comprises lupus nephritis (LN). In some embodiments, the autoimmune disease is lupus nephritis (LN). In some embodiments, the autoimmune disease comprises SLE and LN. In some embodiments, the autoimmune disease is SLE and LN.

Also provided herein is a method of reducing B cells in a subject, the method including administering to a subject having a B cell-mediated disease a composition comprising natural killer (NK) cells genetically engineered to express a chimeric antigen receptor (CAR) that binds to CD19, wherein the CAR comprises: (a) an extracellular antigen-binding domain; (b) a transmembrane domain; and (c) an intracellular signaling domain. Also provided herein is a method of reducing the level of an autoantibody in a subject, the method including administering to a subject having a B cell-mediated disease a composition comprising natural killer (NK) cells genetically engineered to express a chimeric antigen receptor (CAR) that binds to CD19, wherein the CAR comprises: (a) an extracellular antigen-binding domain; (b) a transmembrane domain; and (c) an intracellular signaling domain. In some embodiments, the B cell-mediated disease is an autoimmune disease. In some embodiments, the genetically engineered NK cells are allogeneic to the subject.

Also provided herein is a method of treating systemic lupus erythematosus (SLE), the method including administering to a subject having SLE a composition comprising natural killer (NK) cells genetically engineered to express a chimeric antigen receptor (CAR) that binds to CD19, wherein the CAR comprises: (a) an extracellular antigen-binding domain; (b) a transmembrane domain; and (c) an intracellular signaling domain. Also provided herein is a method of preventing systemic lupus erythematosus (SLE), the method including administering to a subject determined to be at risk of SLE a composition comprising natural killer (NK) cells genetically engineered to express a chimeric antigen receptor (CAR) that binds to CD19, wherein the CAR comprises: (a) an extracellular antigen-binding domain; (b) a transmembrane domain; and (c) an intracellular signaling domain.

Also provided herein is a method of treating lupus nephritis (LN), the method including administering to a subject having LN a composition comprising natural killer (NK) cells genetically engineered to express a chimeric antigen receptor (CAR) that binds to CD19, wherein the CAR comprises: (a) an extracellular antigen-binding domain; (b) a transmembrane domain; and (c) an intracellular signaling domain. Also provided herein is a method of preventing lupus nephritis (LN), the method including administering to a subject determined to be at risk of LN a composition comprising natural killer (NK) cells genetically engineered to express a chimeric antigen receptor (CAR) that binds to CD19, wherein the CAR comprises: (a) an extracellular antigen-binding domain; (b) a transmembrane domain; and (c) an intracellular signaling domain.

In some embodiments, the extracellular antigen-binding domain comprises a heavy chain variable region (VH) having a CDR-1, a CDR-2, and a CDR-3 comprising the amino acid sequences set forth in SEQ ID NOS: 24, 25, and 26, respectively; and a light chain variable region (VL) having a CDR-1, a CDR-2, and a CDR-3 comprising the amino acid sequences set forth in SEQ ID NOS: 27, 28, and 29, respectively. In some embodiments, the VH comprises the amino acid sequence set forth in SEQ ID NO:35. In some embodiments, the VL comprises the amino acid sequence set forth in SEQ ID NO:36. In some embodiments, the VH comprises the amino acid sequence set forth in SEQ ID NO:35, and the VL comprises the amino acid sequence set forth in SEQ ID NO:36. In some embodiments, the extracellular antigen-binding domain is a single-chain variable fragment (scFv) comprising the amino acid sequence set forth in SEQ ID NO: 37.

Also provided herein is a method of treating an autoimmune disease, the method comprising administering to a subject having an autoimmune disease a composition comprising natural killer (NK) cells genetically engineered to express a chimeric antigen receptor (CAR) that binds to CD19, wherein the CAR comprises: (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) having a CDR-1, a CDR-2, and a CDR-3 comprising the amino acid sequences set forth in SEQ ID NOS: 27, 28, and 29, respectively; (b) a transmembrane domain comprising a CD8alpha transmembrane region; and (c) an intracellular signaling domain comprising an intracellular signaling region of OX40 and a CD3zeta domain.

Also provided herein is a method of preventing an autoimmune disease, the method comprising administering to a subject having an autoimmune disease a composition comprising natural killer (NK) cells genetically engineered to express a chimeric antigen receptor (CAR) that binds to CD19, wherein the CAR comprises: (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) having a CDR-1, a CDR-2, and a CDR-3 comprising the amino acid sequences set forth in SEQ ID NOS: 27, 28, and 29, respectively; (b) a transmembrane domain; and (c) an intracellular signaling domain. In some embodiments, the genetically engineered NK cells are allogeneic to the subject.

Also provided herein is a method of preventing an autoimmune disease, the method comprising administering to a subject having an autoimmune disease a composition comprising natural killer (NK) cells genetically engineered to express a chimeric antigen receptor (CAR) that binds to CD19, wherein the genetically engineered NK cells are allogeneic to the subject. In some embodiments, the CAR comprises: (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) having a CDR-1, a CDR-2, and a CDR-3 comprising the amino acid sequences set forth in SEQ ID NOS: 27, 28, and 29, respectively; (b) a transmembrane domain; and (c) an intracellular signaling domain.

Also provided herein is a method of treating an autoimmune disease, the method comprising administering to a subject having an autoimmune disease a composition comprising natural killer (NK) cells genetically engineered to express a chimeric antigen receptor (CAR) that binds to CD19, wherein the genetically engineered NK cells are allogeneic to the subject. In some embodiments, the CAR comprises: (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) having a CDR-1, a CDR-2, and a CDR-3 comprising the amino acid sequences set forth in SEQ ID NOS: 27, 28, and 29, respectively; (b) a transmembrane domain; and (c) an intracellular signaling domain.

Also provided herein is a method of treating an autoimmune disease, the method comprising administering to a subject having an autoimmune disease a composition comprising natural killer (NK) cells genetically engineered to express a chimeric antigen receptor (CAR) that binds to CD19, wherein the CAR comprises: (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) having a CDR-1, a CDR-2, and a CDR-3 comprising the amino acid sequences set forth in SEQ ID NOS: 27, 28, and 29, respectively; (b) a transmembrane domain; and (c) an intracellular signaling domain. In some embodiments, the genetically engineered NK cells are allogeneic to the subject.

Also provided herein is a method of treating an autoimmune disease, the method comprising administering to a subject having an autoimmune disease a composition comprising natural killer (NK) cells genetically engineered to express a chimeric antigen receptor (CAR) that binds to CD19, wherein: (i) prior to administration of the composition comprising NK cells genetically engineered to express a CAR to the subject, the subject has been administered a lymphodepleting therapy; and (ii) the lymphodepleting therapy comprises administration of cyclophosphamide and does not comprise administration of fludarabine; and (iii) the autoimmune disease is selected from the group consisting of scleroderma, myositis, and vasculitis.

Also provided herein is a method of treating an autoimmune disease, the method comprising administering to a subject having an autoimmune disease a composition comprising natural killer (NK) cells genetically engineered to express a chimeric antigen receptor (CAR) that binds to CD19, wherein: (i) the composition comprising the NK cells genetically engineered to express a CAR is administered to the subject in a dosing regimen comprising a dosing cycle, wherein the dosing cycle comprises a first dose, a second dose, and a third dose of the composition; (ii) each of the first, second, and third doses of the dosing cycle comprises between about 1×10⁹ CAR-expressing NK cells and about 2.5×10⁹ CAR-expressing NK cells; (iii) the second dose is administered to the subject about 2-4 days after the first dose is administered to the subject, and the third dose is administered to the subject about 2-4 days after the second dose is administered to the subject; and (iv) about three days prior to administration of the composition comprising NK cells genetically engineered to express a CAR to the subject, the subject is administered a lymphodepleting therapy consisting of a single dose of about 1000 mg/m2 of cyclophosphamide. In some embodiments, each dose of the dosing cycle comprises between about 1×10⁸ CAR-expressing NK cells and about 1×10¹⁰ CAR-expressing NK cells, or between about 3×10⁸ CAR-expressing NK cells and about 3×10⁹ CAR-expressing NK cells, each inclusive. In some embodiments, each dose of the dosing cycle comprises between about 1×10⁸ CAR-expressing NK cells and about 1×10¹⁰ CAR-expressing NK cells, each inclusive. In some embodiments, each dose of the dosing cycle comprises between about 3×10⁸ CAR-expressing NK cells and about 3×10⁹ CAR-expressing NK cells, each inclusive.

In some embodiments, the autoimmune disease is a B cell-mediated disease.

Also provided herein is a method of reducing B cells in a subject, the method comprising administering to a subject having B cell-mediated disease a composition comprising natural killer (NK) cells genetically engineered to express a chimeric antigen receptor (CAR) that binds to CD19, wherein the CAR comprises: (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) having a CDR-1, a CDR-2, and a CDR-3 comprising the amino acid sequences set forth in SEQ ID NOS: 27, 28, and 29, respectively; (b) a transmembrane domain comprising a CD8alpha transmembrane region; and (c) an intracellular signaling domain comprising an intracellular signaling region of OX40 and a CD3zeta domain. Also provided herein is a method of reducing the level of an autoantibody in a subject, the method comprising administering to a subject having B cell-mediated disease a composition comprising natural killer (NK) cells genetically engineered to express a chimeric antigen receptor (CAR) that binds to CD19, wherein the CAR comprises: (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) having a CDR-1, a CDR-2, and a CDR-3 comprising the amino acid sequences set forth in SEQ ID NOS: 27, 28, and 29, respectively; (b) a transmembrane domain comprising a CD8alpha transmembrane region; and (c) an intracellular signaling domain comprising an intracellular signaling region of OX40 and a CD3zeta domain. In some embodiments, the B cell-mediated disease is an autoimmune disease. In some embodiments, the genetically engineered NK cells are allogeneic to the subject.

In some embodiments, the autoimmune disease is a T cell-mediated disease. In some embodiments, the autoimmune disease is a plasma cell-mediated disease. In some embodiments, the subject is seropositive for an autoantibody. In some embodiments, the autoantibody is associated with the autoimmune disease.

In some embodiments, the autoimmune disease comprises scleroderma. In some embodiments, the autoimmune disease is scleroderma. In some embodiments, the autoimmune disease comprises systemic sclerosis (also known as systemic scleroderma). In some embodiments, the autoimmune disease is systemic sclerosis (also known as systemic scleroderma). In some embodiments, the autoimmune disease comprises localized scleroderma. In some embodiments, the autoimmune disease is localized scleroderma.

In some embodiments, the autoimmune disease comprises myositis (also known as IIM). In some embodiments, the autoimmune disease is myositis (also known as IIM). In some embodiments, the autoimmune disease is selected from the group consisting of anti-synthetase syndrome (ASSD), overlap myopathy (OM), dermatomyositis (DM), clinically amyopathic dermatomyositis, juvenile myositis (JM), necrotizing myopathy (NM), polymyositis (PM), and sporadic inclusion body myositis (sIBM). In some embodiments, the autoimmune disease is ASSD. In some embodiments, the autoimmune disease is OM. In some embodiments, the autoimmune disease is DM. In some embodiments, the autoimmune disease is JM. In some embodiments, the autoimmune disease is NM. In some embodiments, the autoimmune disease is PM. In some embodiments, the autoimmune disease is sIBM.

In some embodiments, the autoimmune disease comprises vasculitis. In some embodiments, the autoimmune disease is vasculitis. In some embodiments, the vasculitis is large-vessel vasculitis. In some embodiments, the vasculitis is medium-vessel vasculitis. In some embodiments, the vasculitis is small-vessel vasculitis. In some embodiments, the vasculitis is anti-neutrophilic cytoplasmic autoantibody (ANCA) vasculitis. In some embodiments the ANCA vasculitis is granulomatosis with polyangiitis (GPA). In some embodiments the ANCA vasculitis is microscopic polyangiitis (MPA). In some embodiments the ANCA vasculitis is eosinophilic granulomatosis with polyangiitis (EGPA).

In some embodiments, the autoimmune disease comprises myasthenia gravis (MG). In some embodiments, the autoimmune disease is MG. In some embodiments, MG is ocular MG. In some embodiments, MG is early-onset generalized MG. In some embodiments, MG is late-onset MG.

In some embodiments, the autoimmune disease comprises multiple sclerosis (MS). In some embodiments, the autoimmune disease is MS. In some embodiments, MS is primary progressive MS (PPMS). In some embodiments, MS is secondary-progressive MS (SPMS). In some embodiments, MS is relapsing-remitting MS (RRMS).

In some embodiments, the autoimmune disease comprises systemic lupus erythematosus (SLE). In some embodiments, the autoimmune disease is systemic lupus erythematosus (SLE). In some embodiments, the SLE does not comprise LN. In some embodiments, the autoimmune disease comprises lupus nephritis (LN). In some embodiments, the autoimmune disease is lupus nephritis (LN). In some embodiments, the autoimmune disease comprises SLE and LN. In some embodiments, the autoimmune disease is SLE and LN.

Also provided herein is a method of treating systemic lupus erythematosus (SLE), the method comprising administering to a subject having SLE a composition comprising natural killer (NK) cells genetically engineered to express a chimeric antigen receptor (CAR) that binds to CD19, wherein the CAR comprises: (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively; (b) a transmembrane domain; and (c) an intracellular signaling domain.

Also provided herein is a method of treating lupus nephritis (LN), the method comprising administering to a subject having LN a composition comprising natural killer (NK) cells genetically engineered to express a chimeric antigen receptor (CAR) that binds to CD19, wherein the CAR comprises: (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively; (b) a transmembrane domain; and (c) an intracellular signaling domain.

In some embodiments, the transmembrane domain comprises a CD8alpha hinge. In some embodiments, the transmembrane domain comprises a CD8alpha transmembrane region. In some embodiments, the transmembrane domain comprises a CD8alpha hinge and a CD8alpha transmembrane region. In some embodiments, the intracellular signaling domain comprises an intracellular signaling region of OX40. In some embodiments, the intracellular signaling domain comprises a CD3zeta domain. In some embodiments, the intracellular signaling domain comprises an intracellular signaling region of OX40 and a CD3zeta domain.

In some embodiments, the VH comprises the amino acid sequence set forth in SEQ ID NO:35. In some embodiments, the VL comprises the amino acid sequence set forth in SEQ ID NO: 36. In some embodiments, the VH comprises the amino acid sequence set forth in SEQ ID NO: 35, and the VL comprises the amino acid sequence set forth in SEQ ID NO:36. In some embodiments, the extracellular antigen-binding domain comprises the amino acid sequence set forth in SEQ ID NO:37. In some embodiments, the extracellular antigen-binding domain is an scFv comprising the amino acid sequence set forth in SEQ ID NO:37.

In some embodiments, the CD8alpha hinge comprises the amino acid sequence set forth in SEQ ID NO:6. In some embodiments, the CD8alpha transmembrane region comprises the amino acid sequence set forth in SEQ ID NO:8. In some embodiments, the transmembrane domain comprises the amino acid sequence set forth in SEQ ID NO:6. SEQ ID NO:8, and/or SEQ ID NO: 10. In some embodiments, the transmembrane domain comprises the amino acid sequence set forth in SEQ ID NO:8 or SEQ ID NO:10. In some embodiments, the transmembrane domain comprises the amino acid sequence set forth in SEQ ID NO:8. In some embodiments, the transmembrane domain comprises the amino acid sequence set forth in SEQ ID NO:6 and SEQ ID NO: 8. In some embodiments, the transmembrane domain comprises the amino acid sequence set forth in SEQ ID NO:10. In some embodiments, the intracellular signaling region of OX40 comprises the amino acid sequence set forth in SEQ ID NO: 14. In some embodiments, the CD3zeta domain comprises the amino acid sequence set forth in SEQ ID NO: 16. In some embodiments, the CAR comprises the amino acid sequence set forth in SEQ ID NO:38.

In some embodiments, the NK cells genetically engineered to express a CAR are also engineered to express interleukin-15 (IL15). In some embodiments, the NK cells genetically engineered to express a CAR also express a membrane-bound interleukin-15 (mbIL15). In some embodiments, the mbIL15 comprises the amino acid sequence set forth in SEQ ID NO:22. In some embodiments, the mbIL15 comprises the amino acid sequence set forth in SEQ ID NO:23. In some embodiments, the mbIL15 comprises the amino acid sequence set forth in SEQ ID NO:40. In some embodiments, the CAR and the mbIL15 are bicistronically encoded by the same nucleic acid molecule. In some embodiments, the nucleic acid sequences encoding the CAR and the mbIL15 are separated by a nucleic acid sequence encoding a T2A peptide. In some embodiments, the T2A peptide comprises the amino acid sequence set forth in SEQ ID NO:20.

In some embodiments, the NK cells genetically engineered to express a CAR are also genetically edited. In some embodiments, the NK cells are genetically edited to increase IL15 signaling. In some embodiments, the methods comprise genetically editing the NK cells to increase IL15 signaling. In some embodiments, the NK cells are genetically edited to reduce expression of the CISH gene. In some embodiments, the methods comprise genetically editing the NK cells to reduce expression of the CISH gene. In some embodiments, the NK cells are genetically edited to reduce expression of the Cis protein. In some embodiments, the methods comprise genetically editing the NK cells to reduce expression of the Cis protein. In some embodiments, the NK cells comprise a disruption in one or both alleles of the CISH gene. In some embodiments, the NK cells comprise a disruption in one allele of the CISH gene. In some embodiments, the NK cells comprise a disruption in both alleles of the CISH gene.

In some embodiments, the composition comprising NK cells genetically engineered to express a CAR is administered to the subject in a dosing regimen comprising a dosing cycle. In some embodiments, the dosing cycle comprises a first dose, a second dose, and a third dose of the composition comprising NK cells genetically engineered to express a CAR.

In some embodiments, the second dose is administered to the subject between about 5 days after and about 10 days after the first dose is administered to the subject. In some embodiments, the third dose is administered to the subject between about 5 days after and about 10 days after the second dose is administered to the subject. In some embodiments, the second dose is administered about 7 days after the first dose is administered to the subject. In some embodiments, the third dose is administered about 7 days after the second dose is administered to the subject. In some embodiments, the second dose is administered about 7 days after the first dose is administered to the subject, and the third dose is administered about 7 days after the second dose is administered to the subject.

In some embodiments, the second dose is administered to the subject between about 2 days after and about 4 days after the first dose is administered to the subject. In some embodiments, the third dose is administered to the subject between about 2 days after and about 4 days after the second dose is administered to the subject. In some embodiments, the second dose is administered about 3 days after the first dose is administered to the subject. In some embodiments, the third dose is administered about 4 days after the second dose is administered to the subject. In some embodiments, the second dose is administered about 3 days after the first dose is administered to the subject, and the third dose is administered about 4 days after the second dose is administered to the subject.

In some embodiments, the dosing cycle is between about 21 days and about 49 days, each inclusive. In some embodiments, the dosing cycle is between about 14 days and about 35 days, or between about 21 days and about 28 days, each inclusive. In some embodiments, the dosing cycle is about 14 days. In some embodiments, the dosing cycle is about 21 days. In some embodiments, the dosing cycle is about 28 days. In some embodiments, the dosing cycle is about 35 days. In some embodiments, the dosing cycle is about 42 days. In some embodiments, the dosing cycle is about 42 days. In some embodiments, the dosing cycle is about 42 days. In some embodiments, the dosing cycle is about 49 days.

In some embodiments, the first dose is administered on about Day 0 of the dosing cycle. In some embodiments, the second dose is administered on about Day 7 of the dosing cycle. In some embodiments, the third dose is administered on about Day 14 of the dosing cycle. In some embodiments, the first dose is administered on about Day 0 of the dosing cycle, the second dose is administered on about Day 7 of the dosing cycle, and the third dose is administered on about Day 14 of the dosing cycle.

In some embodiments, the first dose is administered on about Day 0 of the dosing cycle. In some embodiments, the second dose is administered on about Day 2 of the dosing cycle. In some embodiments, the second dose is administered on about Day 3 of the dosing cycle. In some embodiments, the third dose is administered on about Day 4 of the dosing cycle. In some embodiments, the third dose is administered on about Day 5 of the dosing cycle. In some embodiments, the third dose is administered on about Day 6 of the dosing cycle. In some embodiments, the third dose is administered on about Day 7 of the dosing cycle. In some embodiments, each dose is separated by between about 24 hours and about 72 hours. In some embodiments, each dose is separated by at least about 24 hours. In some embodiments, each dose is separated by about 24 hours. In some embodiments, each dose is separated by at least about 48 hours. In some embodiments, each dose is separated by about 48 hours. In some embodiments, each dose is separated by at least about 72 hours. In some embodiments, each dose is separated by about 72 hours.

In some embodiments, the first dose is administered on about Day 0 of the dosing cycle, the second dose is administered on about Day 2 of the dosing cycle, and the third dose is administered on about Day 4 of the dosing cycle. In some embodiments, the first dose is administered on about Day 0 of the dosing cycle, the second dose is administered on about Day 2 of the dosing cycle, and the third dose is administered on about Day 5 of the dosing cycle. In some embodiments, the first dose is administered on about Day 0 of the dosing cycle, the second dose is administered on about Day 3 of the dosing cycle, and the third dose is administered on about Day 5 of the dosing cycle. In some embodiments, the first dose is administered on about Day 0 of the dosing cycle, the second dose is administered on about Day 3 of the dosing cycle, and the third dose is administered on about Day 6 of the dosing cycle. In some embodiments, the first dose is administered on about Day 0 of the dosing cycle, the second dose is administered on about Day 3 of the dosing cycle, and the third dose is administered on about Day 7 of the dosing cycle.

Also provided herein is a method of treating systemic lupus erythematosus (SLE), the method comprising administering to a subject having SLE a composition comprising natural killer (NK) cells genetically engineered to express a chimeric antigen receptor (CAR) that binds to CD19, wherein: (i) the CAR comprises: (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO:36; (b) a transmembrane domain comprising a CD8alpha transmembrane region; and (c) an intracellular signaling domain comprising an intracellular signaling region of OX40 and a CD3zeta domain; (ii) the composition comprising the NK cells genetically engineered to express a CAR is administered to the subject in a dosing regimen comprising a dosing cycle, wherein the dosing cycle comprises a first dose, a second dose, and a third dose of the composition; (iii) each of the first, second, and third doses of the dosing cycle comprises between about 1×10⁸ CAR-expressing NK cells and 2×10⁹ CAR-expressing NK cells; and (iv) the second dose is administered to the subject about 7 days after the first dose is administered to the subject, and the third dose is administered to the subject about 7 days after the second dose is administered to the subject. In some embodiments, each of the first, second, and third doses of the dosing cycle comprises about 1×10⁹ CAR-expressing NK cells, about 1.5×10⁹ CAR-expressing NK cells, about 2×10⁹ CAR-expressing NK cells, or about 2.5×10⁹ CAR-expressing NK cells. In some embodiments, each of the first, second, and third doses of the dosing cycle comprises about 1×10⁹ CAR-expressing NK cells. In some embodiments, each of the first, second, and third doses of the dosing cycle comprises about 1.5×10⁹ CAR-expressing NK cells. In some embodiments, each of the first, second, and third doses of the dosing cycle comprises about 2×10⁹ CAR-expressing NK cells. In some embodiments, the first, second, and third doses of the dosing cycle comprises about 2.5×10⁹ CAR-expressing NK cells.

Also provided herein is a method of treating systemic lupus erythematosus (SLE), the method comprising administering to a subject having SLE a composition comprising natural killer (NK) cells genetically engineered to express a chimeric antigen receptor (CAR) that binds to CD19, wherein: (i) prior to administration of the composition comprising NK cells genetically engineered to express a CAR to the subject, the subject has been administered a lymphodepleting therapy; and (ii) the lymphodepleting therapy comprises administration of cyclophosphamide and does not comprise administration of fludarabine.

Also provided herein is a method of treating lupus nephritis (LN), the method comprising administering to a subject having LN a composition comprising natural killer (NK) cells genetically engineered to express a chimeric antigen receptor (CAR) that binds to CD19, wherein: (i) the CAR comprises: (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO:36; (b) a transmembrane domain comprising a CD8alpha transmembrane region; and (c) an intracellular signaling domain comprising an intracellular signaling region of OX40 and a CD3zeta domain; (ii) the composition comprising the NK cells genetically engineered to express a CAR is administered to the subject in a dosing regimen comprising a dosing cycle, wherein the dosing cycle comprises a first dose, a second dose, and a third dose of the composition; (iii) each of the first, second, and third doses of the dosing cycle comprises between about 1×10⁸ CAR-expressing NK cells and 2×10⁹ CAR-expressing NK cells; and (iv) the second dose is administered to the subject about 7 days after the first dose is administered to the subject, and the third dose is administered to the subject about 7 days after the second dose is administered to the subject. In some embodiments, each of the first, second, and third doses of the dosing cycle comprises about 1×10⁹ CAR-expressing NK cells, about 1.5×10⁹ CAR-expressing NK cells, about 2×10⁹ CAR-expressing NK cells, or about 2.5×10⁹ CAR-expressing NK cells. In some embodiments, each of the first, second, and third doses of the dosing cycle comprises about 1×10⁹ CAR-expressing NK cells. In some embodiments, each of the first, second, and third doses of the dosing cycle comprises about 1.5×10⁹ CAR-expressing NK cells. In some embodiments, each of the first, second, and third doses of the dosing cycle comprises about 2×10⁹ CAR-expressing NK cells. In some embodiments, each of the first, second, and third doses of the dosing cycle comprises about 2.5×10⁹ CAR-expressing NK cells.

Also provided herein is a method of treating lupus nephritis (LN), the method comprising administering to a subject having LN a composition comprising natural killer (NK) cells genetically engineered to express a chimeric antigen receptor (CAR) that binds to CD19, wherein: (i) prior to administration of the composition comprising NK cells genetically engineered to express a CAR to the subject, the subject has been administered a lymphodepleting therapy; and (ii) the lymphodepleting therapy comprises administration of cyclophosphamide and does not comprise administration of fludarabine.

Also provided herein is a method of treating systemic lupus erythematosus (SLE), the method comprising administering to a subject having SLE a composition comprising natural killer (NK) cells genetically engineered to express a chimeric antigen receptor (CAR) that binds to CD19, wherein: (i) the CAR comprises: (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO:36; (b) a transmembrane domain comprising a CD8alpha transmembrane region; and (c) an intracellular signaling domain comprising an intracellular signaling region of OX40 and a CD3zeta domain; (ii) the composition comprising the NK cells genetically engineered to express a CAR is administered to the subject in a dosing regimen comprising a dosing cycle, wherein the dosing cycle comprises a first dose, a second dose, and a third dose of the composition; (iii) each of the first, second, and third doses of the dosing cycle comprises about 2×10⁹ CAR-expressing NK cells or about 2.5×10⁹ CAR-expressing NK cells; and (iv) the second dose is administered to the subject about 3 days after the first dose is administered to the subject, and the third dose is administered to the subject about 4 days after the second dose is administered to the subject.

Also provided herein is a method of treating lupus nephritis (LN), the method comprising administering to a subject having LN a composition comprising natural killer (NK) cells genetically engineered to express a chimeric antigen receptor (CAR) that binds to CD19, wherein: (i) the CAR comprises: (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO:36; (b) a transmembrane domain comprising a CD8alpha transmembrane region; and (c) an intracellular signaling domain comprising an intracellular signaling region of OX40 and a CD3zeta domain; (ii) the composition comprising the NK cells genetically engineered to express a CAR is administered to the subject in a dosing regimen comprising a dosing cycle, wherein the dosing cycle comprises a first dose, a second dose, and a third dose of the composition; (iii) each of the first, second, and third doses of the dosing cycle comprises about 2×10⁹ CAR-expressing NK cells or about 2.5×10⁹ CAR-expressing NK cells; and (iv) the second dose is administered to the subject about 3 days after the first dose is administered to the subject, and the third dose is administered to the subject about 4 days after the second dose is administered to the subject.

In some embodiments, the NK cells genetically engineered to express a CAR also express a membrane-bound interleukin-15 (mbIL15). In some embodiments, the mbIL15 comprises the amino acid sequence set forth in SEQ ID NO:23. In some embodiments, the mbIL15 comprises the amino acid sequence set forth in SEQ ID NO:40. In some embodiments, the CAR and the mbIL15 are bicistronically encoded by the same nucleic acid molecule. In some embodiments, the nucleic acid sequences encoding the CAR and the mbIL15 are separated by a nucleic acid sequence encoding a T2A peptide. In some embodiments, the T2A peptide comprises the amino acid sequence set forth in SEQ ID NO:20.

In some embodiments, the method reduces B cells in the subject. In some embodiments, the method reduces B cells in the subject by at least about 70%, at least about 75%, at least about 80%, at least about 85%, at least about 90%, at least about 91%, at least about 92%, at least about 93%, at least about 94%, at least about 95%, or at least about 99%. In some embodiments, the method reduces B cells in the subject by at least about 70%. In some embodiments, the method reduces B cells in the subject by at least about 75%. In some embodiments, the method reduces B cells in the subject by at least about 80%. In some embodiments, the method reduces B cells in the subject by at least about 85%. In some embodiments, the method reduces B cells in the subject by at least about 90%. In some embodiments, among a plurality of subjects treated according to the method, the number of peripheral B cells in the subjects is reduced by an average of at least about 70%, at least about 75%, at least about 80%, at least about 90%, at least about 95%, or at least about 99%. In some embodiments, among a plurality of subjects treated according to the method, the number of peripheral B cells in the subjects is reduced by an average of at least about 70%. In some embodiments, among a plurality of subjects treated according to the method, the number of peripheral B cells in the subjects is reduced by an average of at least about 75%. In some embodiments, among a plurality of subjects treated according to the method, the number of peripheral B cells in the subjects is reduced by an average of at least about 80%. In some embodiments, among a plurality of subjects treated according to the method, the number of peripheral B cells in the subjects is reduced by an average of at least about 85%. In some embodiments, among a plurality of subjects treated according to the method, the number of peripheral B cells in the subjects is reduced by an average of at least about 90%. In some embodiments, among a plurality of subjects treated according to the method, the number of peripheral B cells in the subjects is reduced by an average of at least about 95%. In some embodiments, among a plurality of subjects treated according to the method, the number of peripheral B cells in the subjects is reduced by an average of at least about 99%.

In some embodiments, the method reduces B cells in the subject for at least about 30 days, at least about 45 days, at least about 60 days, at least about 75 days, or at least about 90 days. In some embodiments, the method reduces B cells in the subject for at least about 30 days. In some embodiments, the method reduces B cells in the subject for at least about 45 days. In some embodiments, the method reduces B cells in the subject for at least about 60 days. In some embodiments, the method reduces B cells in the subject for at least about 75 days. In some embodiments, the method reduces B cells in the subject for at least about 90 days. In some embodiments, the method reduces B cells in the subject for about 30 days, about 45 days, about 60 days, about 75 days, or about 90 days. In some embodiments, the method reduces B cells in the subject for about 30 days. In some embodiments, the method reduces B cells in the subject for about 45 days. In some embodiments, the method reduces B cells in the subject for about 60 days. In some embodiments, the method reduces B cells in the subject for about 75 days. In some embodiments, the method reduces B cells in the subject for about 90 days. In some embodiments, the B cells are peripheral B cells.

In some embodiments, among a plurality of subjects treated according to the method, the number of peripheral B cells in the subjects is significantly reduced for at least about 15 days, at least about 1 month, at least about 2 months, at least about 3 months, at least about 6 months, or at least about 9 months following a final dose of the composition comprising NK cells genetically engineered to express a CAR. In some embodiments, among a plurality of subjects treated according to the method, the number of peripheral B cells in the subjects is significantly reduced for at least about 15 days following a final dose of the composition comprising NK cells genetically engineered to express a CAR. In some embodiments, among a plurality of subjects treated according to the method, the number of peripheral B cells in the subjects is significantly reduced for at least about 1 month following a final dose of the composition comprising NK cells genetically engineered to express a CAR. In some embodiments, among a plurality of subjects treated according to the method, the number of peripheral B cells in the subjects is significantly reduced for at least about 2 months following a final dose of the composition comprising NK cells genetically engineered to express a CAR. In some embodiments, among a plurality of subjects treated according to the method, the number of peripheral B cells in the subjects is significantly reduced for at least about 3 months following a final dose of the composition comprising NK cells genetically engineered to express a CAR. In some embodiments, among a plurality of subjects treated according to the method, the number of peripheral B cells in the subjects is significantly reduced for at least about 6 months following a final dose of the composition comprising NK cells genetically engineered to express a CAR. In some embodiments, among a plurality of subjects treated according to the method, the number of peripheral B cells in the subjects is significantly reduced for at least about 9 months following a final dose of the composition comprising NK cells genetically engineered to express a CAR. In some embodiments, the number of peripheral B cells in the subjects is significantly reduced as compared to subjects not treated according to the method. In some embodiments, the number of peripheral B cells in the subjects is significantly reduced as compared to the subjects prior to administration of a lymphodepleting therapy.

In some embodiments, among a plurality of subjects treated according to the method, the number of peripheral B cells in the subjects is significantly reduced for about 15 days, about 1 month, about 2 months, about 3 months, about 6 months, or about 9 months following a final dose of the composition comprising NK cells genetically engineered to express a CAR. In some embodiments, among a plurality of subjects treated according to the method, the number of peripheral B cells in the subjects is significantly reduced for about 15 days following a final dose of the composition comprising NK cells genetically engineered to express a CAR. In some embodiments, among a plurality of subjects treated according to the method, the number of peripheral B cells in the subjects is significantly reduced for about 1 month following a final dose of the composition comprising NK cells genetically engineered to express a CAR. In some embodiments, among a plurality of subjects treated according to the method, the number of peripheral B cells in the subjects is significantly reduced for about 2 months following a final dose of the composition comprising NK cells genetically engineered to express a CAR. In some embodiments, among a plurality of subjects treated according to the method, the number of peripheral B cells in the subjects is significantly reduced for about 3 months following a final dose of the composition comprising NK cells genetically engineered to express a CAR. In some embodiments, among a plurality of subjects treated according to the method, the number of peripheral B cells in the subjects is significantly reduced for about 6 months following a final dose of the composition comprising NK cells genetically engineered to express a CAR. In some embodiments, among a plurality of subjects treated according to the method, the number of peripheral B cells in the subjects is significantly reduced for about 9 months following a final dose of the composition comprising NK cells genetically engineered to express a CAR.

In some embodiments, the number of peripheral B cells in the subject is significantly reduced within about 10 days, within about 15 days, or within about 30 days after administration of a first dose of the composition comprising NK cells genetically engineered to express a CAR to the subject. In some embodiments, the number of peripheral B cells in the subject is significantly reduced within about 10 days after administration of a first dose of the composition comprising NK cells genetically engineered to express a CAR to the subject. In some embodiments, the number of peripheral B cells in the subject is significantly reduced within about 15 days after administration of a first dose of the composition comprising NK cells genetically engineered to express a CAR to the subject. In some embodiments, the number of peripheral B cells in the subject is significantly reduced within about 30 days after administration of a first dose of the composition comprising NK cells genetically engineered to express a CAR to the subject.

In some embodiments, at about 3 months, at about 6 months, at about 9 months, and/or at about 12 months after administration of a final dose of the composition comprising NK cells genetically engineered to express a CAR to the subject, at least about 30%, at least about 40%, at least about 50%, at least about 60%, at least about 70%, or at least about 80% of the peripheral B cells in the subject are naïve B cells.

In some embodiments, at about 3 months after administration of a final dose of the composition comprising NK cells genetically engineered to express a CAR to the subject, at least about 30% of the peripheral B cells in the subject are naïve B cells. In some embodiments, at about 3 months after administration of a final dose of the composition comprising NK cells genetically engineered to express a CAR to the subject, at least about 40% of the peripheral B cells in the subject are naïve B cells. In some embodiments, at about 3 months after administration of a final dose of the composition comprising NK cells genetically engineered to express a CAR to the subject, at least about 50% of the peripheral B cells in the subject are naïve B cells. In some embodiments, at about 3 months after administration of a final dose of the composition comprising NK cells genetically engineered to express a CAR to the subject, at least about 75% of the peripheral B cells in the subject are naïve B cells.

In some embodiments, at about 6 months after administration of a final dose of the composition comprising NK cells genetically engineered to express a CAR to the subject, at least about 30% of the peripheral B cells in the subject are naïve B cells. In some embodiments, at about 6 months after administration of a final dose of the composition comprising NK cells genetically engineered to express a CAR to the subject, at least about 40% of the peripheral B cells in the subject are naïve B cells. In some embodiments, at about 6 months after administration of a final dose of the composition comprising NK cells genetically engineered to express a CAR to the subject, at least about 50% of the peripheral B cells in the subject are naïve B cells. In some embodiments, at about 6 months after administration of a final dose of the composition comprising NK cells genetically engineered to express a CAR to the subject, at least about 75% of the peripheral B cells in the subject are naïve B cells.

In some embodiments, at about 9 months after administration of a final dose of the composition comprising NK cells genetically engineered to express a CAR to the subject, at least about 30% of the peripheral B cells in the subject are naïve B cells. In some embodiments, at about 9 months after administration of a final dose of the composition comprising NK cells genetically engineered to express a CAR to the subject, at least about 40% of the peripheral B cells in the subject are naïve B cells. In some embodiments, at about 9 months after administration of a final dose of the composition comprising NK cells genetically engineered to express a CAR to the subject, at least about 50% of the peripheral B cells in the subject are naïve B cells. In some embodiments, at about 9 months after administration of a final dose of the composition comprising NK cells genetically engineered to express a CAR to the subject, at least about 75% of the peripheral B cells in the subject are naïve B cells.

In some embodiments, at about 12 months after administration of a final dose of the composition comprising NK cells genetically engineered to express a CAR to the subject, at least about 30% of the peripheral B cells in the subject are naïve B cells. In some embodiments, at about 12 months after administration of a final dose of the composition comprising NK cells genetically engineered to express a CAR to the subject, at least about 40% of the peripheral B cells in the subject are naïve B cells. In some embodiments, at about 12 months after administration of a final dose of the composition comprising NK cells genetically engineered to express a CAR to the subject, at least about 50% of the peripheral B cells in the subject are naïve B cells. In some embodiments, at about 12 months after administration of a final dose of the composition comprising NK cells genetically engineered to express a CAR to the subject, at least about 75% of the peripheral B cells in the subject are naïve B cells.

In some embodiments, the naïve B cells are non-class-switched B cells. In some embodiments, non-class-switched B cells are IgM or IgD isotype. In some embodiments, the non-class-switched B cells are IgM isotype. In some embodiments, the non-class-switched cells are IgD isotype.

In some embodiments, the method reduces the level of an autoantibody in the subject. In some embodiments, the method reduces the level of an autoantibody in the subject by at least about 50%, at least about 60%, at least about 70%, at least about 80%, at least about 90%, at least about 95%, or at least about 99%. In some embodiments, among a plurality of subjects treated according to the method, the level of an autoantibody in the subjects is reduced by an average of at least about 80%, at least about 90%, at least about 95%, or at least about 99%. In some embodiments, among a plurality of subjects treated according to the method, the level of an autoantibody in the subjects is reduced by an average of at least about 50%. In some embodiments, among a plurality of subjects treated according to the method, the level of an autoantibody in the subjects is reduced by an average of at least about 60%. In some embodiments, among a plurality of subjects treated according to the method, the level of an autoantibody in the subjects is reduced by an average of at least about 70%. In some embodiments, among a plurality of subjects treated according to the method, the level of an autoantibody in the subjects is reduced by an average of at least about 80%. In some embodiments, among a plurality of subjects treated according to the method, the level of an autoantibody in the subjects is reduced by an average of at least about 90%. In some embodiments, among a plurality of subjects treated according to the method, the level of an autoantibody in the subjects is reduced by an average of at least about 95%. In some embodiments, among a plurality of subjects treated according to the method, the level of an autoantibody in the subjects is reduced by an average of at least about 99%. In some embodiments, the level of an autoantibody in the subjects is significantly reduced as compared to subjects having the disease or condition and not treated according to the method. In some embodiments, the level of an autoantibody in the subjects is significantly reduced as compared to the subjects prior to administration of the composition comprising NK cells genetically engineered to express a CAR.

Also provided herein is use of a composition comprising natural killer (NK) cells genetically engineered to express a chimeric antigen receptor (CAR) that binds to CD19 for reducing B cells in a subject with a B cell-mediated disease, wherein: the CAR comprises: (a) an extracellular antigen-binding domain; (b) a transmembrane domain; and (c) an intracellular signaling domain. In some embodiments, the B cell-mediated disease is an autoimmune disease. In some embodiments, prior to administration of the composition comprising NK cells genetically engineered to express a CAR to the subject, the subject has been administered a lymphodepleting therapy. In some embodiments, the lymphodepleting therapy does not comprise administration of fludarabine. In some embodiments, the lymphodepleting therapy comprises administration of cyclophosphamide and does not comprise administration of fludarabine.

Also provide herein is use of a lymphodepleting therapy for the preparation of a subject having an autoimmune disease for treatment with a composition comprising natural killer (NK) cells genetically engineered to express a chimeric antigen receptor (CAR) that binds to CD19, wherein: (i) the lymphodepleting therapy is administered to the subject prior to administration of the composition to the subject; and (ii) the lymphodepleting therapy comprises cyclophosphamide and does not comprise fludarabine.

Also provided herein is use of a composition comprising natural killer (NK) cells genetically engineered to express a chimeric antigen receptor (CAR) that binds to CD19 for treating a subject with an autoimmune disease, wherein: the CAR comprises: (a) an extracellular antigen-binding domain; (b) a transmembrane domain; and (c) an intracellular signaling domain. In some embodiments, prior to administration of the composition comprising NK cells genetically engineered to express a CAR to the subject, the subject has been administered a lymphodepleting therapy. In some embodiments, the lymphodepleting therapy does not comprise administration of fludarabine. In some embodiments, the lymphodepleting therapy comprises administration of cyclophosphamide and does not comprise administration of fludarabine.

In some embodiments, the genetically engineered NK cells are allogeneic to the subject. In some embodiments, the autoimmune disease is a B cell-mediated autoimmune disease. In some embodiments, the autoimmune disease is selected from the group consisting of systemic lupus erythematosus (SLE), lupus nephritis (LN), scleroderma, rheumatoid arthritis (RA), myasthenia gravis (MG), multiple sclerosis (MS), NMDA/NMDAR encephalitis, transverse myelitis, neuromyelitis optica spectrum disorder (NMOSD), myelin oligodendrocyte glycoprotein antibody disease (MOGAD), myelin oligodendrocyte glycoprotein spectrum disorder (MOGSD), idiopathic inflammatory myopathy (IIM: also known as myositis), Sjogren's disease, pemphigus vulgaris, bullous pemphigoid (BP), membranous nephropathy (MN), immune thrombocytopenia (ITP), Hashimoto's disease, Grave's disease, insulin resistance, type I diabetes, antiphospholipid syndrome, vasculitis, anti-neutrophilic cytoplasmic antibodies (ANCA) vasculitis (AAV), and anti-synthetase syndrome (ASSD). In some embodiments, the autoimmune disease is selected from the group consisting of SLE, LN, scleroderma, MG, myositis (also known as IIM), and vasculitis. In some embodiments, the autoimmune disease is SLE. In some embodiments, the autoimmune disease is LN. In some embodiments, the autoimmune disease is scleroderma. In some embodiments, the autoimmune disease is MG. In some embodiments, the autoimmune disease is IIM. In some embodiments, the autoimmune disease is vasculitis. In some embodiments, the autoimmune disease is MS. In some embodiments, the autoimmune disease is NMDA/NMDAR encephalitis. In some embodiments, the autoimmune disease is transverse myelitis. In some embodiments, the autoimmune disease is NMOSD. In some embodiments, the autoimmune disease is MOGAD. In some embodiments, the autoimmune disease is MOGSD. In some embodiments, the autoimmune disease is Sjogren's disease. In some embodiments, the autoimmune disease is pemphigus vulgaris. In some embodiments, the autoimmune disease is BP. In some embodiments, the autoimmune disease is MN. In some embodiments, the autoimmune disease is ITP. In some embodiments, the autoimmune disease is Hasmimoto's disease. In some embodiments, the autoimmune disease is Grave's disease. In some embodiments, the autoimmune disease is type 1 diabetes. In some embodiments, the autoimmune disease is antiphospholipid syndrome.

Also provided herein is use of a composition comprising natural killer (NK) cells genetically engineered to express a chimeric antigen receptor (CAR) that binds to CD19 for treating a subject with systemic lupus erythematosus (SLE), wherein the CAR comprises: (a) an extracellular antigen-binding domain; (b) a transmembrane domain; and (c) an intracellular signaling domain.

Also provided herein is use of a composition comprising natural killer (NK) cells genetically engineered to express a chimeric antigen receptor (CAR) that binds to CD19 for treating a subject with lupus nephritis (LN), wherein the CAR comprises: (a) an extracellular antigen-binding domain; (b) a transmembrane domain; and (c) an intracellular signaling domain.

Also provided herein is use of a composition comprising natural killer (NK) cells genetically engineered to express a chimeric antigen receptor (CAR) that binds to CD19 for treating a subject having an autoimmune disease, wherein the CAR comprises: (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively; (b) a transmembrane domain comprising a CD8alpha transmembrane region; and (c) an intracellular signaling domain comprising an intracellular signaling region of OX40 and a CD3zeta domain.

Also provided herein is use of a composition comprising natural killer (NK) cells genetically engineered to express a chimeric antigen receptor (CAR) that binds to CD19 for treating a subject with systemic lupus erythematosus (SLE), wherein the CAR comprises: (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively; (b) a transmembrane domain; and (c) an intracellular signaling domain.

Also provided herein is use of a composition comprising natural killer (NK) cells genetically engineered to express a chimeric antigen receptor (CAR) that binds to CD19 for treating a subject with lupus nephritis (LN), wherein the CAR comprises: (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively; (b) a transmembrane domain; and (c) an intracellular signaling domain.

Also provided herein is use of a composition comprising natural killer (NK) cells genetically engineered to express a chimeric antigen receptor (CAR) that binds to CD19 for treating a subject having systemic lupus erythematosus (SLE), wherein: (i) the CAR comprises: (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO:36; (b) a transmembrane domain comprising a CD8alpha transmembrane region; and (c) an intracellular signaling domain comprising an intracellular signaling region of OX40 and a CD3zeta domain; (ii) the composition comprising the NK cells genetically engineered to express a CAR is formulated for administration in a dosing regimen comprising a dosing cycle, wherein the dosing cycle comprises a first dose, a second dose, and a third dose of the composition; (iii) each of the first, second, and third doses of the dosing cycle comprises between about 1×10⁸ CAR-expressing NK cells and 2×10⁹ CAR-expressing NK cells; and (iv) the second dose is for administration to the subject about 7 days after the first dose is administered to the subject, and the third dose is for administration to the subject about 7 days after the second dose is administered to the subject.

Also provided herein is use of a composition comprising natural killer (NK) cells genetically engineered to express a chimeric antigen receptor (CAR) that binds to CD19 for treating a subject having lupus nephritis (LN), wherein: (i) the CAR comprises: (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO:36; (b) a transmembrane domain comprising a CD8alpha transmembrane region; and (c) an intracellular signaling domain comprising an intracellular signaling region of OX40 and a CD3zeta domain; (ii) the composition comprising the NK cells genetically engineered to express a CAR is formulated for administration in a dosing regimen comprising a dosing cycle, wherein the dosing cycle comprises a first dose, a second dose, and a third dose of the composition; (iii) each of the first, second, and third doses of the dosing cycle comprises between about 1×10⁸ CAR-expressing NK cells and 3×10⁹ CAR-expressing NK cells; and (iv) the second dose is for administration to the subject about 7 days after the first dose is administered to the subject, and the third dose is for administration to the subject about 7 days after the second dose is administered to the subject.

Also provided herein is use of a composition comprising natural killer (NK) cells genetically engineered to express a chimeric antigen receptor (CAR) that binds to CD19 for treating a subject having systemic lupus erythematosus (SLE), wherein: (i) the CAR comprises: (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO:36; (b) a transmembrane domain comprising a CD8alpha transmembrane region; and (c) an intracellular signaling domain comprising an intracellular signaling region of OX40 and a CD3zeta domain; (ii) the composition comprising the NK cells genetically engineered to express a CAR is formulated for administration in a dosing regimen comprising a dosing cycle, wherein the dosing cycle comprises a first dose, a second dose, and a third dose of the composition; (iii) each of the first, second, and third doses of the dosing cycle comprises about 2×10⁹ CAR-expressing NK cells or about 2.5×10⁹ CAR-expressing NK cells; and (iv) the second dose is for administration to the subject about 3 days after the first dose is administered to the subject, and the third dose is for administration to the subject about 4 days after the second dose is administered to the subject.

Also provided herein is use of a composition comprising natural killer (NK) cells genetically engineered to express a chimeric antigen receptor (CAR) that binds to CD19 for treating a subject having lupus nephritis (LN), wherein: (i) the CAR comprises: (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO:36; (b) a transmembrane domain comprising a CD8alpha transmembrane region; and (c) an intracellular signaling domain comprising an intracellular signaling region of OX40 and a CD3zeta domain; (ii) the composition comprising the NK cells genetically engineered to express a CAR is formulated for administration in a dosing regimen comprising a dosing cycle, wherein the dosing cycle comprises a first dose, a second dose, and a third dose of the composition; (iii) each of the first, second, and third doses of the dosing cycle comprises about 2×10⁹ CAR-expressing NK cells or about 2×10⁹ CAR-expressing NK cells; and (iv) the second dose is for administration to the subject about 3 days after the first dose is administered to the subject, and the third dose is for administration to the subject about 4 days after the second dose is administered to the subject.

Also provided herein is use of a composition comprising natural killer (NK) cells genetically engineered to express a chimeric antigen receptor (CAR) that binds to CD19 to reduce B cells in a subject having a B cell-mediated disease, wherein: (i) the composition comprising the NK cells genetically engineered to express a CAR is for administration to the subject in a dosing regimen comprising a dosing cycle; and (ii) the composition reduces peripheral B cells in the subject by at least about 90%; peripheral B cells are significantly reduced in the subject for the duration of the dosing cycle; and/or at least about 75% of repopulating peripheral B cells are non-class-switched B cells. In some embodiments, the composition reduces peripheral B cells in the subject by at least about 90%. In some embodiments, peripheral B cells are significantly reduced in the subject for the duration of the dosing cycle. In some embodiments, at least about 75% of repopulating peripheral B cells are non-class-switched B cells.

Also provided herein is a kit comprising (i) a composition comprising natural killer (NK) cells genetically engineered to express a chimeric antigen receptor (CAR) that binds to CD19; and (ii) instructions for administering the composition to a subject having a B cell-mediated disease. In some embodiments, the B cell-mediated disease is an autoimmune disease.

Also provided herein is a kit comprising (i) a composition comprising natural killer (NK) cells genetically engineered to express a chimeric antigen receptor (CAR) that binds to CD19; and (ii) instructions for administering the composition to a subject having an autoimmune disease.

In some embodiments, the genetically engineered NK cells are allogeneic to the subject. In some embodiments the CAR comprises (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) having a CDR-1, a CDR-2, and a CDR-3 comprising the amino acid sequences set forth in SEQ ID NOS: 27, 28, and 29, respectively; (b) a transmembrane domain; and (c) an intracellular signaling domain. In some embodiments, administering the composition to the subject comprises administration of the composition to the subject in a dosing regimen comprising a dosing cycle. In some embodiments, the dosing cycle comprises a first dose, a second dose, and a third dose of the composition.

In some embodiments, each of the first, second, and third doses of the dosing cycle comprises about 2×10⁹ CAR-expressing NK cells. In some embodiments, each of the first, second, and third doses of the dosing cycle comprises about 2.5×10⁹ CAR-expressing NK cells.

In some embodiments, each dose of the dosing cycle comprises between about 1×10⁸ CAR-expressing NK cells and about 1×10¹⁰ CAR-expressing NK cells. In some embodiments, each dose of the dosing cycle comprises between about 3×10⁸ CAR-expressing NK cells and about 3×10⁹ CAR-expressing NK cells. In some embodiments, each dose of the dosing cycle comprises about 1×10⁸ CAR-expressing NK cells. In some embodiments, each dose of the dosing cycle comprises about 3×10⁸ CAR-expressing NK cells. In some embodiments, each dose of the dosing cycle comprises about 5×10⁸ CAR-expressing NK cells. In some embodiments, each dose of the dosing cycle comprises about 1×10⁹ CAR-expressing NK cells. In some embodiments, each dose of the dosing cycle comprises about 1.25×10⁹ CAR-expressing NK cells. In some embodiments, each dose of the dosing cycle comprises about 1.5×10⁹ CAR-expressing NK cells. In some embodiments, each dose of the dosing cycle comprises about 1.75×10⁹ CAR-expressing NK cells. In some embodiments, each dose of the dosing cycle comprises about 2×10⁹ CAR-expressing NK cells. In some embodiments, each dose of the dosing cycle comprises about 2.5×10⁹ CAR-expressing NK cells. In some embodiments, each dose of the dosing cycle comprises about 3×10⁹ CAR-expressing NK cells.

In some embodiments, each dose of the dosing cycle comprises between about 1×10⁶ CAR-expressing NK cells/kilogram (kg) and about 1×10⁸ CAR-expressing NK cells/kg. In some embodiments, if the subject weighs less than 50 kilograms, each dose of the dosing cycle comprises between about 1×10⁶ CAR-expressing NK cells/kg and about 1×10⁸ CAR-expressing NK cells/kg. In some embodiments, each dose of the dosing cycle comprises about 1×10⁶ CAR-expressing NK cells/kg. In some embodiments, each dose of the dosing cycle comprises about 2×10⁶ CAR-expressing NK cells/kg. In some embodiments, each dose of the dosing cycle comprises about 3×10⁶ CAR-expressing NK cells/kg. In some embodiments, each dose of the dosing cycle comprises about 4×10⁶ CAR-expressing NK cells/kg. In some embodiments, each dose of the dosing cycle comprises about 5×10⁶ CAR-expressing NK cells/kg. In some embodiments, each dose of the dosing cycle comprises about 6×10⁶ CAR-expressing NK cells/kg. In some embodiments, each dose of the dosing cycle comprises about 7×10⁶ CAR-expressing NK cells/kg. In some embodiments, each dose of the dosing cycle comprises about 8×10⁶ CAR-expressing NK cells/kg. In some embodiments, each dose of the dosing cycle comprises about 9×10⁶ CAR-expressing NK cells/kg. In some embodiments, each dose of the dosing cycle comprises about 1×10⁷ CAR-expressing NK cells/kg. In some embodiments, each dose of the dosing cycle comprises about 2×10⁷ CAR-expressing NK cells/kg. In some embodiments, each dose of the dosing cycle comprises about 3×10⁷ CAR-expressing NK cells/kg. In some embodiments, each dose of the dosing cycle comprises about 4×10⁷ CAR-expressing NK cells/kg. In some embodiments, each dose of the dosing cycle comprises about 5×10⁷ CAR-expressing NK cells/kg. In some embodiments, each dose of the dosing cycle comprises about 6×10⁷ CAR-expressing NK cells/kg. In some embodiments, each dose of the dosing cycle comprises about 7×10⁷ CAR-expressing NK cells/kg. In some embodiments, each dose of the dosing cycle comprises about 8×10⁷ CAR-expressing NK cells/kg. In some embodiments, each dose of the dosing cycle comprises about 9×10⁷ CAR-expressing NK cells/kg. In some embodiments, each dose of the dosing cycle comprises about 1×10⁸ CAR-expressing NK cells/kg.

In some embodiments, the NK cells genetically engineered to express a CAR are allogeneic to the subject. In some embodiments, the NK cells are obtained from a donor that does not have a B cell-mediated disease. In some embodiments, the NK cells are obtained from a donor that does not have an autoimmune disease. In some embodiments, the NK cells are obtained from a donor that does not have SLE. In some embodiments, the NK cells are obtained from a donor that does not have LN.

In some embodiments, the NK cells genetically engineered to express a CD19 CAR also express a CAR that binds to an antigen associated with an autoimmune disease. In some embodiments, the composition further comprises immune cells genetically engineered to express a CAR that binds to an antigen associated with an autoimmune disease. In some embodiments, the antigen is selected from the group consisting of BAFF-R, BCMA, CD20, CD22, CD27, CD28, CD33, CD38, CD45, CD47, CD54, CD56, CD81, CD117, CD138, CD200, FcRH5, GPRC5D, and SLAMF7. In some embodiments, the antigen is BAFF-R. In some embodiments, the antigen is BCMA. In some embodiments, the antigen is CD20. In some embodiments, the antigen is CD22. In some embodiments, the antigen is CD27. In some embodiments, the antigen is CD28. In some embodiments, the antigen is CD38. In some embodiments, the antigen is CD45. In some embodiments, the antigen is CD47. In some embodiments, the antigen is CD54. In some embodiments, the antigen is CD56. In some embodiments, the antigen is CD81. In some embodiments, the antigen is CD117. In some embodiments, the antigen is CD138. In some embodiments, the antigen is CD200. In some embodiments, the antigen is FcRH5. In some embodiments, the antigen is GPRC5D. In some embodiments, the antigen is SLAMF7. In some embodiments, the immune cells comprise NK cells. In some embodiments, the immune cells comprise T cells. In some embodiments, the immune cells comprise NK cells and T cells.

In some embodiments, the method further comprises administering a lymphodepleting therapy to the subject prior to administration of the composition comprising NK cells genetically engineered to express a CAR. In some embodiments, prior to administration of the composition comprising NK cells genetically engineered to express a CAR to the subject, the subject has been administered a lymphodepleting therapy.

In some embodiments, the lymphodepleting therapy comprises administration of cyclophosphamide. In some embodiments, the lymphodepleting therapy does not comprise administration of fludarabine. In some embodiments, the lymphodepleting therapy comprises administration of cyclophosphamide and does not comprise administration of fludarabine.

Also provided herein is a method of preparing a subject having an autoimmune disease for treatment with a composition comprising natural killer (NK) cells genetically engineered to express a chimeric antigen receptor (CAR) that binds to CD19, the method comprising administering a lymphodepleting therapy to the subject prior to administration of the composition to the subject, wherein the lymphodepleting therapy consists of cyclophosphamide.

Also provided herein is a method of treating or preventing an autoimmune disease, the method comprising administering to a subject having or suspected or having, or determined to be at risk of, or at risk of relapse of, an autoimmune disease, a composition comprising natural killer (NK) cells genetically engineered to express a chimeric antigen receptor (CAR) that binds to CD19, wherein: (i) the CAR comprises: (a) an extracellular antigen-binding domain; (b) a transmembrane domain; and (c) an intracellular signaling domain; (ii) prior to administration of the composition comprising NK cells genetically engineered to express a CAR to the subject, the subject has been administered a lymphodepleting therapy; and (iii) the lymphodepleting therapy comprises administration of cyclophosphamide and does not comprise administration of fludarabine.

In some embodiments, the NK cells genetically engineered to express a CAR also express a mbIL15. In some embodiments, the method is a method of treating an autoimmune disease. In some embodiments, the method is a method of preventing an autoimmune disease. In some embodiments, the subject has an autoimmune disease. In some embodiments, the subject has been determined to be at risk of an autoimmune disease. In some embodiments, the subject has been determined to be at risk of relapse of an autoimmune disease. In some embodiments, the genetically engineered NK cells are allogeneic to the subject.

In some embodiments, the lymphodepleting therapy comprises administration of cyclophosphamide at between about 200 mg/m² and about 600 mg/m² daily. In some embodiments, the lymphodepleting therapy comprises administration of cyclophosphamide at about 200 mg/m² daily. In some embodiments, the lymphodepleting therapy comprises administration of cyclophosphamide at about 300 mg/m² daily. In some embodiments, the lymphodepleting therapy comprises administration of cyclophosphamide at about 400 mg/m² daily. In some embodiments, the lymphodepleting therapy comprises administration of cyclophosphamide at about 500 mg/m² daily. In some embodiments, the lymphodepleting therapy comprises administration of cyclophosphamide at about 600 mg/m² daily. In some embodiments, the lymphodepleting therapy comprises administration of cyclophosphamide daily for 2-4 days. In some embodiments, the lymphodepleting therapy comprises administration of cyclophosphamide daily for 3 days. In some embodiments, the lymphodepleting therapy comprises administration of cyclophosphamide at about 500 mg/m² daily for 3 days. In some embodiments, the lymphodepleting therapy comprises administration of cyclophosphamide at about 500 mg/m² daily on each of days −5, −4, and −3.

In some embodiments, the lymphodepleting therapy comprises administration of a single dose of cyclophosphamide. In some embodiments, a single dose of cyclophosphamide comprises between about 500 mg/m² and about 1500 mg/m² cyclophosphamide. In some embodiments, a single dose of cyclophosphamide comprises about 500 mg/m² cyclophosphamide. In some embodiments, a single dose of cyclophosphamide comprises about 750 mg/m² cyclophosphamide. In some embodiments, a single dose of cyclophosphamide comprises about 1000 mg/m² cyclophosphamide. In some embodiments, a single dose of cyclophosphamide comprises about 1250 mg/m² cyclophosphamide. In some embodiments, a single dose of cyclophosphamide comprises about 1500 mg/m² cyclophosphamide. In some embodiments, a single dose of cyclophosphamide is administered about 3 days prior to administration of the composition comprising NK cells genetically engineered to express a CAR. In some embodiments, a single dose of about 1000 mg/m² cyclophosphamide is administered about 3 days prior to administration of the composition comprising NK cells genetically engineered to express a CAR. In some embodiments, a single dose of about 1000 mg/m² cyclophosphamide is administered on day −3.

In some embodiments, the lymphodepleting therapy comprises administration of fludarabine. In some embodiments, the lymphodepleting therapy comprises administration of fludarabine at between about 20 mg/m² and about 40 mg/m² daily. In some embodiments, the lymphodepleting therapy comprises administration of fludarabine at about 20 mg/m² daily. In some embodiments, the lymphodepleting therapy comprises administration of fludarabine at about 25 mg/m² daily. In some embodiments, the lymphodepleting therapy comprises administration of fludarabine at about 30 mg/m² daily. In some embodiments, the lymphodepleting therapy comprises administration of fludarabine at about 40 mg/m² daily. In some embodiments, the lymphodepleting therapy comprises administration of fludarabine daily for 2-4 days. In some embodiments, the lymphodepleting therapy comprises administration of fludarabine daily for 3 days. In some embodiments, the lymphodepleting therapy comprises administration of 25 mg/m² fludarabine daily for 3 days. In some embodiments, the lymphodepleting therapy comprises administration of 25 mg/m² fludarabine daily on each of 5, 4, and 3 days prior to administration of the composition comprising NK cells genetically engineered to express a CAR. In some embodiments, the lymphodepleting therapy comprises administration of 25 mg/m² fludarabine daily on each of days −5, −4, and −3.

In some embodiments, the lymphodepleting therapy comprises administration of cyclophosphamide and fludarabine. In some embodiments, the lymphodepleting therapy comprises administration of cyclophosphamide at about 300 mg/m² daily and fludarabine at about 30 mg/m² daily, each for 3 days. In some embodiments, the lymphodepleting therapy comprises administration of cyclophosphamide at about 500 mg/m² daily and fludarabine at about 30 mg/m² daily, each for 3 days. In some embodiments, the lymphodepleting therapy comprises administration of about 300 mg/m² of cyclophosphamide and about 30 mg/m² of fludarabine on each of Days −5, −4, and −3. In some embodiments, the lymphodepleting therapy comprises administration of about 500 mg/m² of cyclophosphamide and about 30 mg/m² of fludarabine on each of Days −5, −4, and −3. In some embodiments, the lymphodepleting therapy comprises administration of a single dose of about 1000 mg/m² cyclophosphamide and three daily doses of about 25 mg/m² fludarabine. In some embodiments, the lymphodepleting therapy comprises administration of a single dose of about 1000 mg/m² cyclophosphamide about 3 days prior to administration of the composition comprises NK cells genetically engineered to express a CAR and administration of a dose of about 25 mg/m² fludarabine on each of 5, 4, and 3 days prior to administration of the composition comprising NK cells genetically engineered to express a CAR. In some embodiments, the lymphodepleting therapy comprises administration of a single dose of about 1000 mg/m² cyclophosphamide on day-3 and a dose of about 25 mg/m² fludarabine on each of days −5, −4, and −3.

In some embodiments, the method comprises administering a corticosteroid to the subject before, during, and/or after administration of the lymphodepleting therapy. In some embodiments, the subject is administered a corticosteroid before, during, and/or after administration of the lymphodepleting therapy. In some embodiments, the subject is administered a corticosteroid before administration of the lymphodepleting therapy. In some embodiments, the subject is administered a corticosteroid during administration of the lymphodepleting therapy. In some embodiments, the subject is administered a corticosteroid after administration of the lymphodepleting therapy. In some embodiments, the subject is administered a corticosteroid before, during, and after administration of the lymphodepleting therapy. In some embodiments, the corticosteroid comprises a glucocorticoid. In some embodiments the corticosteroid is or comprises prednisone.

In some embodiments, the method comprises administering a corticosteroid to the subject before, during, and/or after administration of the composition. In some embodiments, the subject is administered a corticosteroid before, during, and/or after administration of the composition. In some embodiments, the subject is administered a corticosteroid before administration of the composition. In some embodiments, the subject is administered a corticosteroid during administration of the composition. In some embodiments, the subject is administered a corticosteroid after administration of the composition. In some embodiments, the subject is administered a corticosteroid before, during, and after administration of the composition. In some embodiments, the corticosteroid comprises a glucocorticoid. In some embodiments the corticosteroid is or comprises prednisone.

In some embodiments, the method comprises administering an immunosuppressive agent to the subject before, during, and/or after administration of the lymphodepleting therapy. In some embodiments, the subject is administered an immunosuppressive agent before, during, and/or after administration of the lymphodepleting therapy. In some embodiments, the subject is administered an immunosuppressive agent before administration of the lymphodepleting therapy. In some embodiments, the subject is administered an immunosuppressive agent during administration of the lymphodepleting therapy. In some embodiments, the subject is administered an immunosuppressive agent after administration of the lymphodepleting therapy. In some embodiments, the subject is administered an immunosuppressive agent before, during, and after administration of the lymphodepleting therapy.

In some embodiments, the method comprises administering an immunosuppressive agent to the subject before, during, and/or after administration of the composition. In some embodiments, the subject is administered an immunosuppressive agent before, during, and/or after administration of the composition. In some embodiments, the subject is administered an immunosuppressive agent before administration of the composition. In some embodiments, the subject is administered an immunosuppressive agent during administration of the composition. In some embodiments, the subject is administered an immunosuppressive agent after administration of the composition. In some embodiments, the subject is administered an immunosuppressive agent before, during, and after administration of the composition.

In some embodiments, the immunosuppressive agent comprises an antithymocyte globulin (ATG), an inhibitor of mammalian target of rapamycin (mTOR), a calcineurin inhibitor, or any combination thereof. In some embodiments, the immunosuppressive agent is an antithymocyte globulin (ATG). In some embodiments, the immunosuppressive agent is an inhibitor of mammalian target of rapamycin (mTOR). In some embodiments, the immunosuppressive agent is a calcineurin inhibitor (e.g., voclosporin).

In some embodiments, the subject was diagnosed with the autoimmune disease (e.g., SLE or LN) between at least about 18 weeks and at least about 30 weeks prior to administration of the composition. In some embodiments, the subject was diagnosed with the autoimmune disease (e.g., SLE or LN) between about 18 weeks and about 30 weeks prior to administration of the composition. In some embodiments, the subject was diagnosed with the autoimmune disease at least about 18 weeks, at least about 20 weeks, at least about 22 weeks, at least about 24 weeks, at least about 26 weeks, at least about 28 weeks, or at least about 30 weeks prior to administration of the composition. In some embodiments, the subject was diagnosed with the autoimmune disease at least about 20 weeks prior to administration of the composition. In some embodiments, the subject was diagnosed with the autoimmune disease at least about 21 weeks prior to administration of the composition. In some embodiments, the subject was diagnosed with the autoimmune disease at least about 22 weeks prior to administration of the composition. In some embodiments, the subject was diagnosed with the autoimmune disease at least about 23 weeks prior to administration of the composition. In some embodiments, the subject was diagnosed with the autoimmune disease at least about 24 weeks prior to administration of the composition. In some embodiments, the subject was diagnosed with the autoimmune disease at least about 25 weeks prior to administration of the composition. In some embodiments, the subject was diagnosed with the autoimmune disease at least about 26 weeks prior to administration of the composition. In some embodiments, the subject was diagnosed with the autoimmune disease at least about 27 weeks prior to administration of the composition. In some embodiments, the subject was diagnosed with the autoimmune disease at least about 28 weeks prior to administration of the composition. In some embodiments, the subject was diagnosed with the autoimmune disease at least about 29 weeks prior to administration of the composition. In some embodiments, the subject was diagnosed with the autoimmune disease at least about 30 weeks prior to administration of the composition.

In some embodiments, the subject is a human. In some embodiments, the subject is less than 18 years of age. In some embodiments, the subject is between about 12 years of age and about 18 years of age. In some embodiments, the subject is at least 12 years of age. In some embodiments, the subject is between about 14 years of age and about 18 years of age. In some embodiments, the subject is at least 14 years of age. In some embodiments, the subject is between about 16 years of age and about 18 years of age. In some embodiments, the subject is at least 16 years of age. In some embodiments, the subject is an adult. In some embodiments, the subject is at least 18 years of age. In some embodiments, the subject is between about 18 and 65 years of age.

In some embodiments, the autoimmune disease is relapsed/refractory. In some embodiments, the subject has relapsed following treatment with and/or is refractory to a prior line of therapy for the autoimmune disease. In some embodiments, the subject has relapsed following treatment with a prior line of therapy for the autoimmune disease. In some embodiments, the subject is refractory to a prior line of therapy for the autoimmune disease.

In some embodiments, the subject does not have lupus nephritis (LN). In some embodiments, the subject has lupus nephritis (LN). In some embodiments, at the time of administration of the composition to the subject, the subject has SLE with active LN. In some embodiments, the LN is refractory LN. In some embodiments, refractory LN is LN that has failed to respond to at least 2 prior lines of therapy. In some embodiments, at the time of administration of the composition to the subject, the subject has active LN.

In some embodiments, the subject does not have neuropsychiatric systemic lupus erythematosus (NPSLE). In some embodiments, the subject has neuropsychiatric systemic lupus erythematosus (NPSLE). In some embodiments, the subject has SLE without renal involvement.

In some embodiments, the SLE is relapsed/refractory SLE. In some embodiments, the subject has relapsed following treatment with and/or is refractory to a prior line of therapy for SLE. In some embodiments, the subject has relapsed following treatment with a prior line of therapy for SLE. In some embodiments, the subject is refractory to a prior line of therapy for SLE. In some embodiments, the subject has relapsed following treatment with and is refractory to a prior line of therapy for SLE.

In some embodiments, the prior line of therapy comprises two, three, or four prior lines of therapy. In some embodiments, the prior line of therapy comprises two prior lines of therapy. In some embodiments, the prior line of therapy comprises three prior lines of therapy. In some embodiments, the prior line of therapy comprises four prior lines of therapy. In some embodiments, the subject has previously received at least two prior lines of therapy for LN. In some embodiments, the subject has previously received at least three prior lines of therapy for LN.

In some embodiments, the at least two prior lines of therapy for LN comprise an immunosuppressant and/or an immunomodulatory agent. In some embodiments, the at least two prior lines of therapy for LN comprise an immunosuppressant. In some embodiments, the at least two prior lines of therapy for LN comprise an immunomodulatory agent. In some embodiments, the at least two prior lines of therapy for LN comprise an immunosuppressant and an immunomodulatory agent. In some embodiments, the at least three prior lines of therapy for LN comprise an immunosuppressant and/or an immunomodulatory agent. In some embodiments, the at least three prior lines of therapy for LN comprise an immunosuppressant. In some embodiments, the at least three prior lines of therapy for LN comprise an immunomodulatory agent. In some embodiments, the at least three prior lines of therapy for LN comprise an immunosuppressant and an immunomodulatory agent.

In some embodiments, the subject was treated with a prior line of therapy for at least about two months. In some embodiments, the subject was treated with a prior line of therapy for between about 3 months and about 24 months. In some embodiments, the subject did not achieve a partial response to the prior line of therapy. In some embodiments, the subject did not achieve a complete response to the prior line of therapy.

In some embodiments, the prior line of therapy comprises a corticosteroid, an immunosuppressive agent, an antimalarial agent, a B cell-targeting agent, hematopoietic stem cell transplant (HSCT), or any combination thereof. In some embodiments, the prior line of therapy comprises a corticosteroid. In some embodiments, the prior line of therapy comprises a glucocorticoid. In some embodiments, the prior line of therapy comprises an antimalarial agent. In some embodiments, the prior line of therapy comprises an immunosuppressive agent. In some embodiments, the prior line of therapy comprises a B cell-targeting agent. In some embodiments, the prior line of therapy comprises hematopoietic stem cell transplant (HSCT). In some embodiments, the prior line of therapy does not comprise HSCT.

In some embodiments, the subject achieves a clinical response following a dosing cycle. In some embodiments, the subject achieves a complete response (CR) following a dosing cycle. In some embodiments, the subject achieves a complete renal response (CRR) following a dosing cycle. In some embodiments, the subject achieves clinical remission following a dosing cycle. In some embodiments, the subject achieves a partial response (PR) following a dosing cycle. In some embodiments, the subject achieves a partial renal response (PRR) following a dosing cycle. In some embodiments, the subject achieves a reduction in disease activity following a dosing cycle. In some embodiments, the subject achieves a reduction in the level of an autoantibody following a dosing cycle. In some embodiments, the autoantibody is associated with the autoimmune disease.

In some embodiments, if the subject exhibits a clinical response to the treatment, the dosing regimen comprises an additional dosing cycle. In some embodiments, if the subject exhibits a partial response to the treatment, the dosing regimen comprises an additional dosing cycle. In some embodiments, if the subject exhibits a complete response to the treatment, the dosing regimen comprises an additional dosing cycle. In some embodiments, if the subject exhibits an initial clinical response to the treatment and subsequently relapses, the dosing regimen comprises an additional dosing cycle. In some embodiments, if the subject exhibits an initial clinical response to the treatment and subsequent disease progression, the dosing regimen comprises an additional dosing cycle. In some embodiments, the dosing regimen comprises two, three, four, or five dosing cycles. In some embodiments, the dosing regimen consists of two, three, four, or five dosing cycles. In some embodiments, the dosing regimen consists of two dosing cycles. In some embodiments, the dosing regimen consists of three dosing cycles. In some embodiments, the dosing regimen consists of four dosing cycles. In some embodiments, the dosing regimen consists of five dosing cycles. In some embodiments, the dosing regimen comprises no more than five dosing cycles.

In some embodiments, the method treats an autoimmune disease. In some embodiments, the method prevents an autoimmune disease. In some embodiments, among a plurality of subjects treated according to the method, the average time between disease flares is reduced as compared to a plurality of subjects having the autoimmune disease and not treated according to the method. In some embodiments, among a plurality of subjects treated according to the method, the average severity of disease flare is reduced as compared to a plurality of subjects having the autoimmune disease and not treated according to the method.

In some embodiments, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% of subjects treated according to the method exhibit a clinical response. In some embodiments, at least about 50% of subjects treated according to the method exhibit a clinical response. In some embodiments, at least about 60% of subjects treated according to the method exhibit a clinical response. In some embodiments, at least about 70% of subjects treated according to the method exhibit a clinical response. In some embodiments, at least about 80% of subjects treated according to the method exhibit a clinical response. In some embodiments, at least about 90% of subjects treated according to the method exhibit a clinical response. In some embodiments, at least about 95% of subjects treated according to the method exhibit a clinical response. In some embodiments, a clinical response comprises a partial response (PR). In some embodiments, a clinical response comprises a complete response (CR). In some embodiments, a clinical response comprises a partial renal response (PRR). In some embodiments, a clinical response comprises a complete renal response (CRR). In some embodiments, a clinical response comprises a reduction in disease activity. In some embodiments, the reduction is disease activity is assessed by a disease index.

In some embodiments, the subject is human. In some embodiments, the subject is at least about 12 years of age. In some embodiments, the subject is at least about 14 years of age. In some embodiments, the subject is at least about 16 years of age. In some embodiments, the subject is an adult. In some embodiments, the subject is at least about 18 years of age.

### BRIEF DESCRIPTION OF THE DRA WINGS

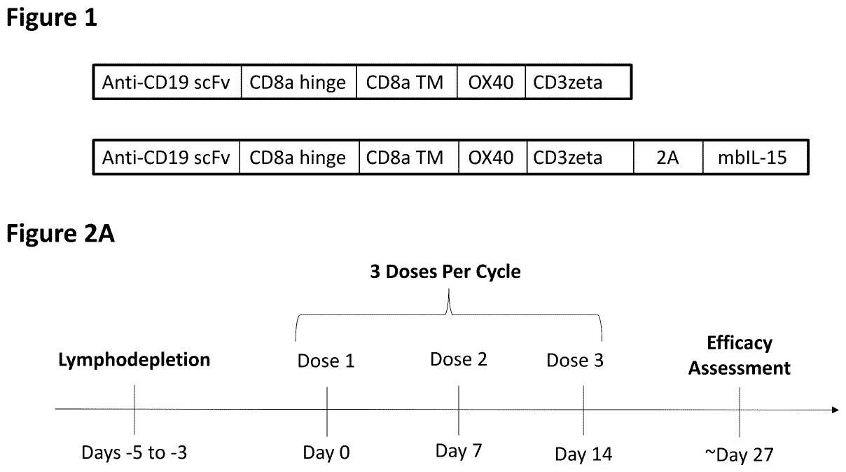

FIG. 1 depicts non-limiting schematics of CD19-directed chimeric antigen receptors (CARs).

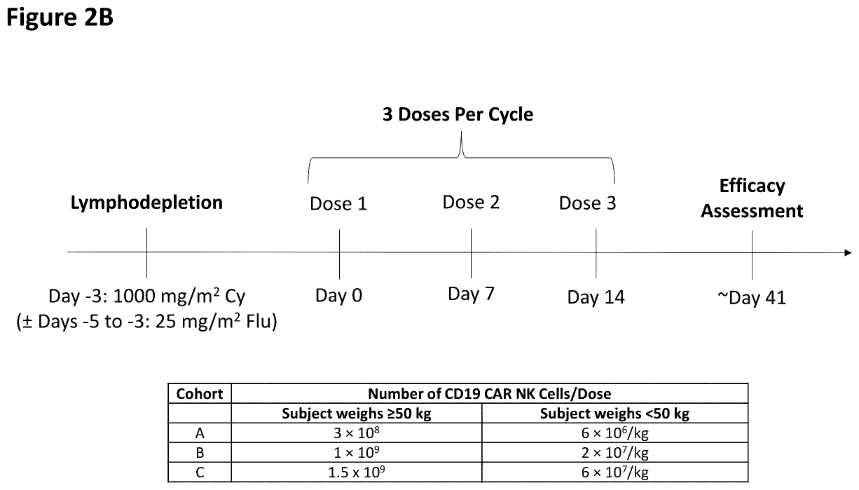

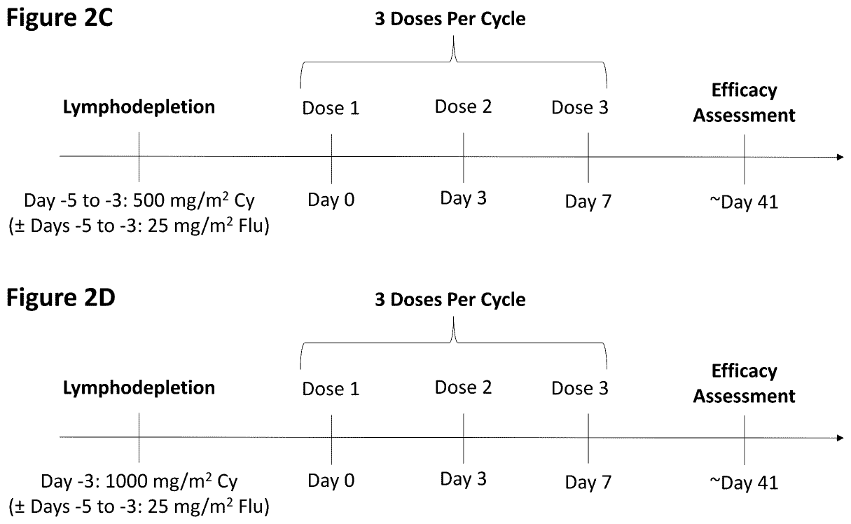

FIGS. 2A-2D depict non-limiting schematics of a dosing cycle for treating an autoimmune disease (e.g., SLE) with CD19 CAR-expressing NK cells (“CD19 CAR NK cells”).

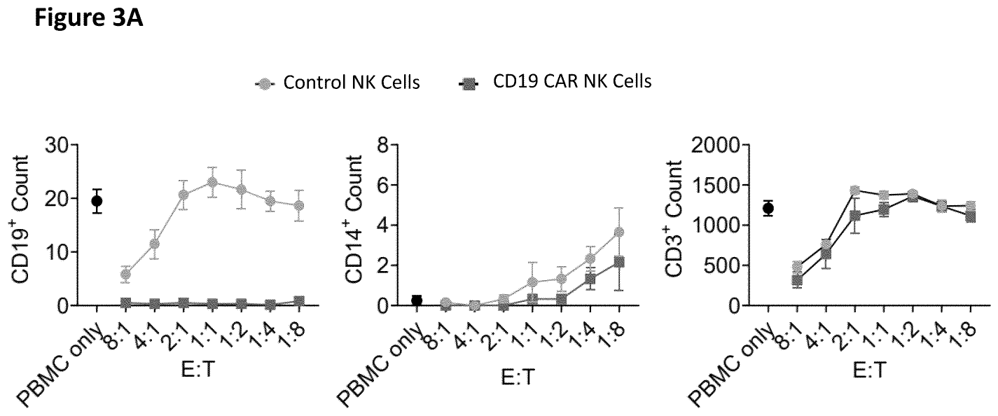

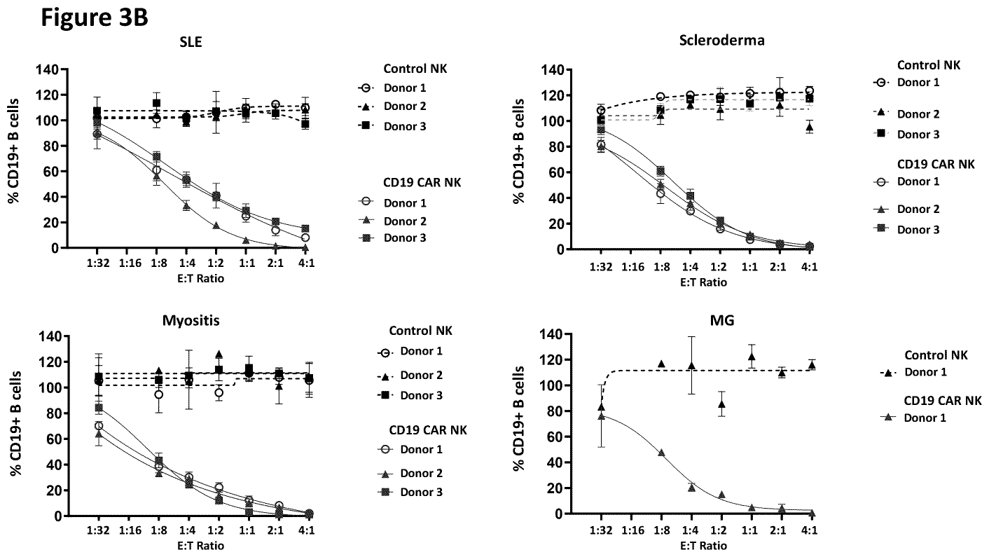

FIG. 3A depicts the in vitro cytotoxicity of nontransduced (“control”) NK cells and CD19 CAR NK cells against CD19+, CD14+, and CD3+ subpopulations of PBMCs.

FIG. 3B depicts the ex vivo cytotoxicity of nontransduced (“control”) NK cells and CD19 CAR NK cells against CD19+ B cells from donors with systemic lupus erythematosus (SLE; n=3), scleroderma (n=3), myositis (n=3), and myasthenia gravis (MG; n=1).

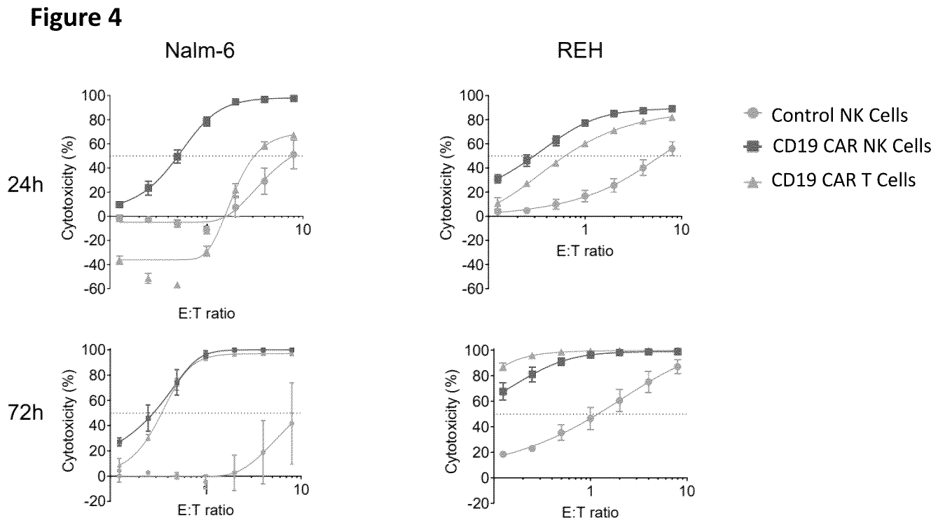

FIG. 4 depicts the in vitro cytotoxicity of control NK cells, CD19 CAR NK cells, and CD19 CAR-expressing T cells (“CD19 CAR T cells”) against CD19+ target cells (Nalm6 and REH cell lines) during 24 or 72 hours of co-culture.

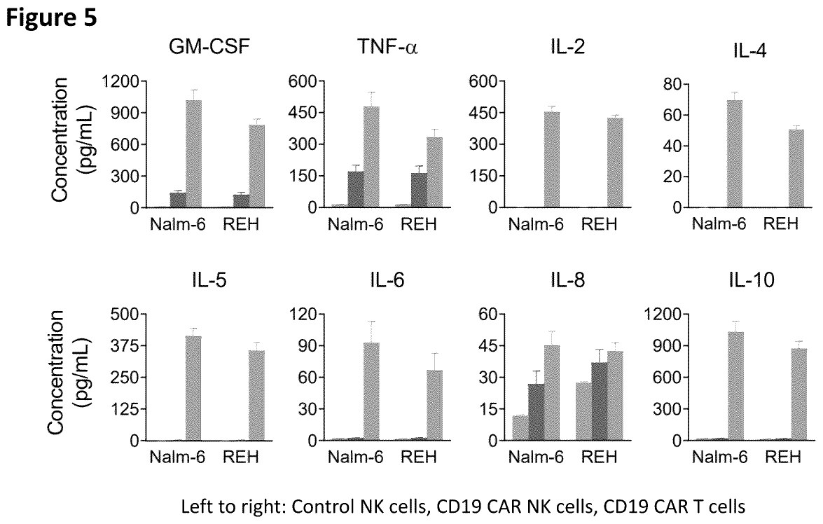

FIG. 5 depicts cytokine levels from control NK cells, CD19 CAR NK cells, and CD19 CAR T cells following 24-hour co-culture with Nalm-6 or REH target cells at a 1:1 E:T ratio (from left to right: control NK cells, CD19 CAR NK cells, CD19 CAR T cells).

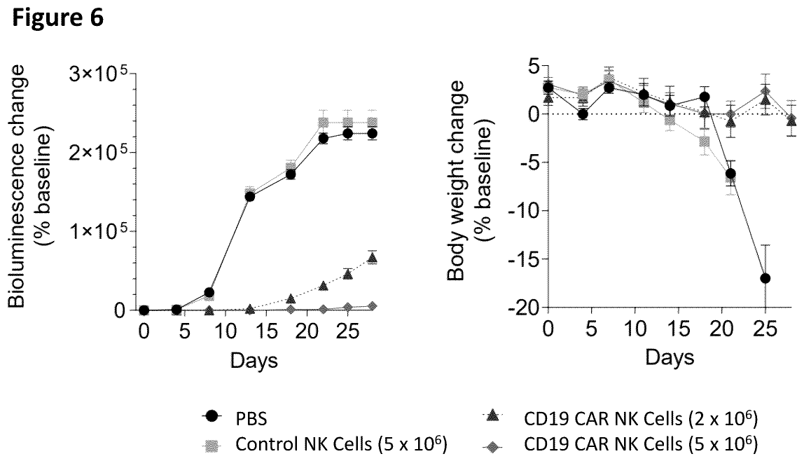

FIG. 6 depicts tumor burden (left) and body weight (right) in a murine CD19-positive xenograft tumor model following treatment with control NK or CD19 CAR NK cells.

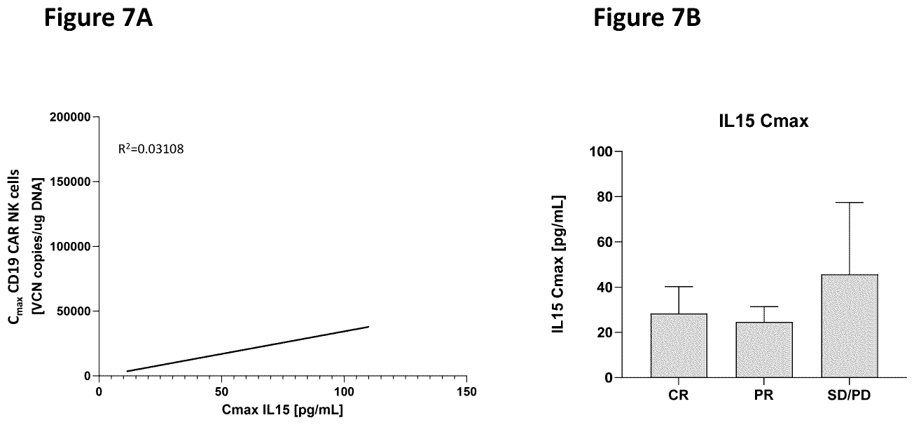

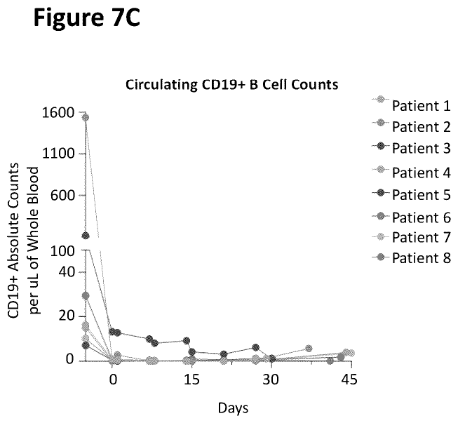

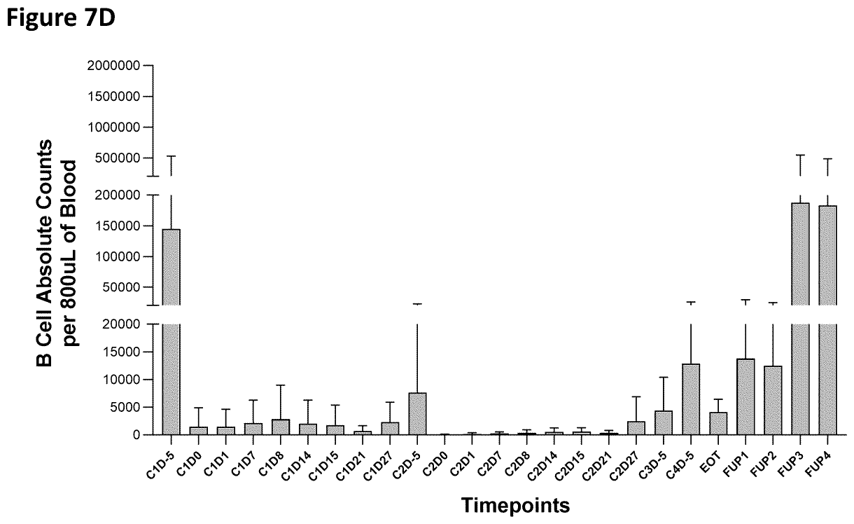

FIG. 7A depicts the peak concentration (Cₘₐₓ) of interleukin 15 (IL15) vs. the Cₘₐₓ of CD19 CAR NK cells in subjects with CD19+ B cell malignancies who were treated with CD19 CAR NK cells in accordance with a non-limiting dosing regimen.

FIG. 7B depicts the average IL15 Cₘₐₓ in subjects achieving complete response (CR), partial response (PR), or stable or progressive disease (SD/PD) in subjects with CD19+ B cell malignancies who were treated with CD19 CAR NK cells in accordance with a non-limiting dosing regimen.

FIG. 7C depicts the number of CD19+ cells per microliter (uL) of whole blood in subjects with CD19+ B cell malignancies who were treated with CD19 CAR NK cells on Days 0, 7, and 14 in accordance with a non-limiting dosing regimen (each line represents a different subject).

FIG. 7D shows the absolute number of B cells per 800 uL whole blood in NHL subjects at baseline (C1D-5) and at the indicated days of one or more dosing cycles with CD19 CAR NK cells (C1: first dosing cycle; C2: second dosing cycle; C3: third dosing cycle; C4: fourth dosing cycle; EOT: end of treatment; FUP1: follow-up one; FUP2: follow-up two; FUP3: follow-up three; FUP4: follow-up four).

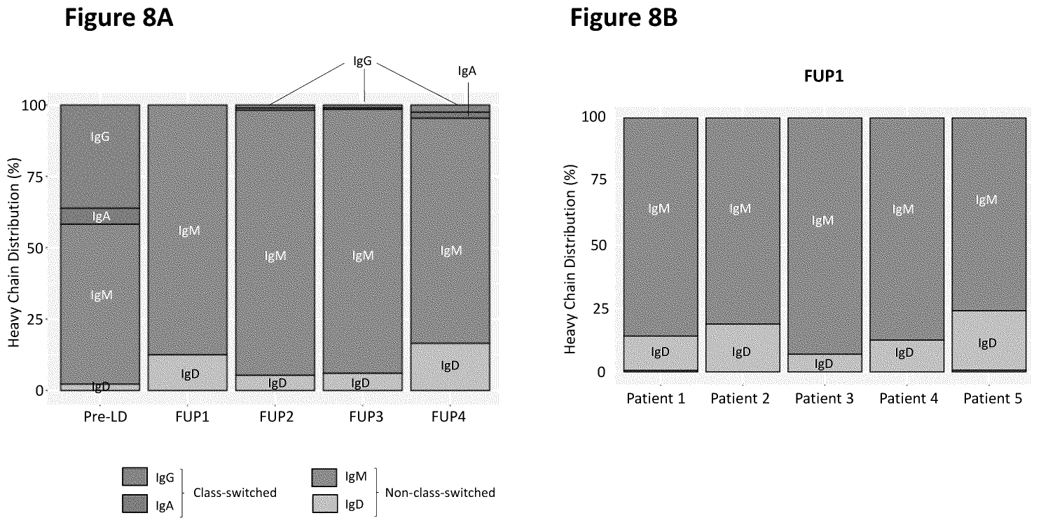

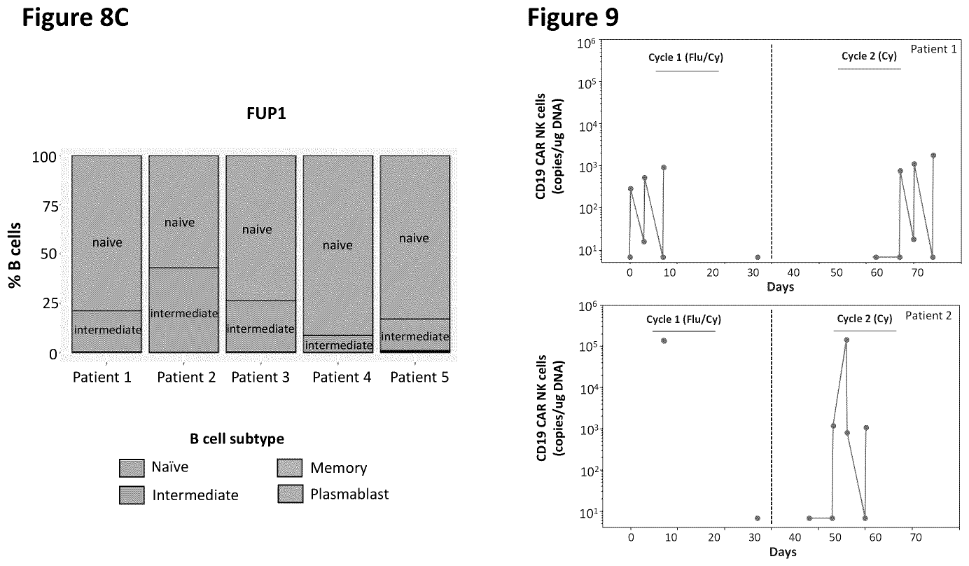

FIG. 8A shows B cell receptor (BCR) heavy chain isotypes in a representative NHL subject prior to lymphodepletion (pre-LD) and at FUP1, FUP2, FUP3, and FUP4, as assessed by mRNA sequencing (n=1).

FIG. 8B shows B cell receptor (BCR) heavy chain isotypes in NHL subjects at FUP1, as assessed by mRNA sequencing (n=5).

FIG. 8C shows the percentage of B cell subtypes in NHL subjects at FUP1, as assessed by transcriptomic analysis (n=5).

FIG. 9 shows the concentration of CD19 CAR NK cells in two subjects with CD19+ B cell malignancies who were administered a lymphodepleting therapy of cyclophosphamide and fludarabine (cy/flu) prior to a first dosing cycle and a lymphodepleting therapy of cyclophosphamide only (cy) prior to a second dosing cycle.

### DETAILED DESCRIPTION

Provided are methods and uses of genetically engineered immune cells and/or compositions thereof, for the treatment of subjects having an autoimmune disease (e.g., lupus). Also provided are methods and uses of genetically engineered immune cells and/or compositions thereof, for the prevention of an autoimmune disease, in a subject suspected of having, or determined to be at risk for, an autoimmune disease. In particular embodiments of any of the provided methods, natural killer (NK) cells are genetically engineered to express a chimeric antigen receptor (CAR) that is directed against CD19.

In some aspects, the autoimmune disease is a B cell-. T cell-, and/or plasma cell-mediated disease. In some aspects, the autoimmune disease is a B cell-mediated disease. For example, in some aspects, the autoimmune disease is systemic lupus erythematosus (SLE), rheumatoid arthritis (RA), and/or multiple sclerosis (MS). In some aspects, the autoimmune disease is acquired immunodeficiency syndrome (AIDS), Addison's disease, alopecia areata, vasculitis (e.g., anti-neutrophilic cytoplasmic antibodies (ANCA) vasculitis), antiphospholipid syndrome, antisynthetase syndrome, atherosclerosis, bullous pemphigoid (BP), celiac disease, chronic inflammatory demyelinating polyneuropathy (CIDP), Graves' disease, Guillain-Barre syndrome, Hashimoto thyroiditis, immune thrombocytopenia (ITP), inflammatory bowel disease (IBD) such as Crohn's disease or ulcerative colitis, insulin resistance, membranous nephropathy (MN), myasthenia gravis (MG), myelin oligodendrocyte glycoprotein antibody disease (MOGAD), myelin oligodendrocyte glycoprotein spectrum disorder (MOGSD), myocardial aneurysm, myocardial infarction, IIM (including anti-synthetase syndrome, dermatomyositis, juvenile myositis, necrotizing myopathy, polymyositis, and sporadic inclusion body myositis), neuromyelitis optica spectrum disorder (NMOSD), NMDA/NMDAR encephalitis, pemphigus vulgaris, pernicious anemia, psoriasis, psoriatic arthritis, reactive arthritis, scleroderma (e.g. localized or systemic scleroderma), Sjögren's disease, transverse myelitis, and/or Type I diabetes.

In some embodiments, the methods and uses include administering to a subject having a B cell-mediated disease (e.g., an autoimmune disease) NK cells genetically engineered to express a recombinant receptor (e.g., a CAR) that binds to an antigen (e.g., CD19) expressed by, associated with, and/or specific to B cells (e.g., CD19). Thus, in some embodiments, the methods and uses include administering to a subject having an autoimmune disease (e.g., SLE) NK cells genetically engineered to express a recombinant receptor (e.g., a CAR) that binds to an antigen (e.g., CD19) expressed by, associated with, and/or specific to cells (e.g., B cells) involved in pathogenesis of the autoimmune disease. The NK cells are generally administered in a composition formulated for administration; the methods generally involve administering the CAR-expressing NK cells as part of a dosing cycle. In some aspects, a dosing cycle comprises multiple (e.g., three) doses of the genetically engineered NK cells. In some aspects, the subject is administered a lymphodepleting therapy prior to administration of the genetically engineered NK cells. Where a subject is administered more than one dosing cycle, the subject may be administered the lymphodepleting therapy prior to administration of each dosing cycle.

Without wishing to be bound by theory, it is contemplated that the methods and uses described herein provide or achieve improved response and/or more durable response or efficacy and/or a reduced risk of toxicity or other side effects, including as compared to alternative methods for treating such autoimmune disease. For example, and as described further below, it is contemplated that the methods provided herein are advantageous by virtue of producing an increased and/or a more durable response as compared to other methods such as B cell-targeting agents (e.g., anti-BAFF, anti-CD19, anti-CD20 antibodies, and/or anti-CD22 antibodies). Also, without wishing to be bound by theory, it is contemplated that the provided methods may be advantageous by having reduced risk of toxicity and/or increased ability to retreat as compared to alternative methods, such as CAR T cell therapies.

CD19 is a glycoprotein expressed at high levels by B cells throughout all stages of B-cell differentiation (Jin et al., Cell Mol Immunol (2020) 18:1896-1093). Further, CD19 is not expressed on hematopoietic stem cells or on any normal tissue apart from those of the B-cell lineage. B cells are thought to play a central role in the pathogenesis of autoimmune diseases, such as rheumatoid arthritis (RA), multiple sclerosis (MS), and SLE.

SLE is considered an incurable disease, characterized by a loss of self-tolerance with autoantibody production, cellular-tissue infiltration, and end-organ damage that can lead to serious organ complications and even death (Doglio et al., J Allergy Clin Immunol (2022) 150 (6): 1289-1301). In particular, hyperactivation of autoreactive B cells is observed in SLE pathogenesis, where it induces plasma cells to produce large amounts of autoantibodies that subsequently circulate and form immune complexes with encountered self-antigens and complement. These immune complexes may then be deposited in small vessels or distal sites, where they can eventually cause organ destruction or dysfunction. See Jin et al. (2020). Among the most significant manifestations of SLE is lupus nephritis (LN). Multiple mechanisms contribute to renal damage in LN, and nephrotic-range proteinuria is found in up to 50% of cases (Parikh et al., Am J Kidney Dis. (2020) 76 (2): 265-81). LN is a major risk factor for morbidity and mortality and affects over half of patients with SLE within 10 years. LN leads to end stage renal disease (ESRD) in 10% of patients and corresponds to a 12% mortality (Almaani et al., Clin J Am Soc Nephrol (2017) 12 (5): 825-35; Hahn et al., Arthritis Care Res (2012) 64 (6): 797-808).

B-cell depletion strategies, in addition to nonsteroidal anti-inflammatory drugs (NSAIDS), antimalarial drugs, glucocorticoids, and immunosuppression, have been investigated for treatment of SLE. B cell-targeting agents such as anti-BAFF antibodies (e.g., belimumab) were found to only partially deplete B cells in SLE patients. Other BAFF blocking agents, as well as the anti-CD20 antibody rituximab, have yielded negative or mixed results in clinical trials. See Jin et al. (2020). For example, several studies have shown that memory B cells can escape depletion by rituximab, whereas rituximab-treated patients with complete B cell depletion have better responses than those with only partial depletion (Schett et al., Lancet (2023) S0140-6736 (23) 01126-1).

Options for patients who have treatment refractory SLE are limited. Hematopoietic cell transplantation (HCT) has also been explored. Unfortunately, this approach has had inconsistent success and comes with potential for considerable toxicity (de Silva et al., Allergy Asthma Clin Immunol (2019) 15:59). Autologous HCT can induce remission in some patients, but the treatment-related mortality exceeds 10% in some studies from complications such as bleeding and infection (Jayne et al., Lupus. (2004) 13 (3): 168-76). Because autologous HCT may be beneficial for selected patients, but not for others, allogeneic HCT has also been evaluated. While potentially increasing disease control, allogeneic HCT has potential for even higher treatment-related mortality, with some reports as high as 20% at two years (Daikeler et al., Bone Marrow Transplant (2009) 44 (1): 27-33). In summary, there is an urgent need for new therapies with the potential to limit toxicity for patients with systemic autoimmune diseases such as SLE, especially those with severe disease and those with LN.

Though T cells are widely considered to be major contributors to inflammatory demyelination in multiple sclerosis (MS), growing evidence suggests a significant role for B cells in disease pathogenesis. Both antibody-dependent and independent mechanisms are thought to underlie B-cell mediated central nervous system (CNS) injury in MS. B cell actions may contribute to both MS relapses and disease progression. Primary progressive MS (PPMS), which affects 10-15% of MS patients, has been a notoriously difficult form of MS to recognize and to treat. Rituximab was tested in PPMS patients in a Phase 2/3 trial but failed to meet the primary endpoint (Comi et al., Annal. Neurol. (2021) 89 (1): 13-23). Another anti-CD20 antibody, ocrelizumab, is the first and only approved treatment for PPMS and recommended as first-line therapy by the ECTRIMS-EAN (European Committee for Treatment and Research in Multiple Sclerosis-European Academy of Neurology; Montalban et al., Eur J Neurol. (2018) 25 (2): 215-37) guidelines. Despite this, the need for more effective therapies in MS, including PPMS, remains.

Rheumatoid arthritis (RA) is a chronic systemic inflammatory disease marked by persistent symmetric polyarthritis (synovitis) affecting primarily small joints. Significant extra-articular involvement may also occur in organs such as the skin, heart, lungs, and eyes (Muzes and Sipos, Cells (2023) 12 (11): 1534). A prominent participation of B cells in RA has been appreciated since the discovery of rheumatoid factor (RF) and has been re-highlighted over the past several years; RF and anti-cyclic-citrillunated peptide (anti-CCP) autoantibodies are well-established indicators of disease and disease severity. Initially based on the idea that RF-producing B cells could perpetuate themselves and induce production of TNF, B cell depletion was hypothesized have a beneficial impact in patients with RA. Transient B cell depletion with rituximab, which is approved from TNF-refractory RA, can ameliorate disease for a prolonged period but typically not indefinitely (Marston et al., Curr. Opin. Rheumatol. (2010) 22 (2): 307-15). Thus, and as some patients do not adequately respond to rituximab treatment, additional therapeutic strategies for treating RA, including TNF refractory RA, are needed.

B cells have been implicated in a number of other autoimmune diseases, including scleroderma (Kraaij and van Laar, Biologics (2008) 2 (3): 389-95), myositis (Oddis and Aggarwal, Nat. Rev. Rheumatol. (2018) 14:279-89), myasthenia gravis (MG; Wu et al., Front. Neurol. (2020) 11:593431), and vasculitis (Merino-Vico et al., Int J Mol Sci. (2022) 23 (1): 387).

Scleroderma is a rare and chronic, autoimmune connective tissue disorder that is primarily characterized by thickening and hardening of the skin and other tissues. The two primary types of scleroderma are systemic scleroderma (also known as systemic sclerosis; SSc) and localized scleroderma. In systemic scleroderma, internal organs such as the digestive tract, heart, lungs, and kidneys may be affected. Depending on how systemic scleroderma manifests, treatment can include immunosuppressive drugs, cyclophosphamide, mycophenolate mofetil, calcium channel blocks (for Raynaud's phenomenon), prokinetic agents and proton pump inhibitors (for esophageal involvement), ACE inhibitors (for renal involvement), and corticosteroids. The CD20 monoclonal antibody (mAb) rituximab has been evaluated in several clinical studies and is used in practice for the management of cutaneous and pulmonary manifestations of SSc but is not approved for this indication. Limitations of rituximab include its action in triggering B cell activating factor (BAFF) secretion, which perpetuates autoreactive B cells, and failure to target autoreactive long-lived plasma cells (Benfaremo & Gabrielli, 2019; Ehrenstein & Wing. 2016). Autologous CD19-CAR T cell therapy was evaluated in four subjects with severe refractory SSc (Muller et al., N Engl J Med (2024) 390 (8): 687-700). The treatment was generally well-tolerated, and the three subjects with ≥6 months of follow up data had decrease in EUSTAR and MRSS scores. In localized scleroderma, the skin is the main organ system involved and muscles and bones may or may not be affected. There are two main forms of localized scleroderma: morphea and linear scleroderma. Morphea is the most common form and presents as a single or multiple plaques, whereas linear scleroderma presents as thickened and indurated skin bands most often on the face or extremities. Different clinical forms of localized scleroderma can coexist in the same patient. Treatments for localized scleroderma can include systemic or topical steroids (e.g., corticosteroids), methotrexate, and phototherapy. There is no curative treatment for scleroderma, such that treatment is designed to relieve symptoms and slow disease progression. The use of different immunosuppressive drugs remains disappointing. (Odonwodo A, Badri T, Hariz A. Scleroderma. [Updated 2022 Aug. 1]. In: StatPearls [Internet]. Treasure Island (FL): StatPearls Publishing; 2023 January. Available from ncbi.nlm.nih.gov/books/NBK537335/).

Idiopathic inflammatory myopathies (IIM, also known as myositis) are heterogenous and rare, and only a few large treatment trial results are available to guide clinicians. A common feature of myositis is chronic inflammation of skeletal muscle, leading to muscle weakness, though other organs such as skin, joints, lungs, gastrointestinal tract, and heart are frequently affected as well. Myositis can be subclassified into anti-synthetase syndrome, polymyositis, dermatomyositis, inclusion body myositis, and immune-mediated necrotizing myopathy (Lundberg et al., Nat. Rev. Rheumatol. (2018) 14:269-78). Conventional therapies for myositis include glucocorticoids and immunosuppressive agents, but biological therapies are increasingly being used. Treatment of myositis with rituximab has yielded mixed results, with the primary end point in the largest clinical trial not being met, despite most patients meeting the definition of improvement (DOI) by the end of the trial (Oddis and Aggarwal 2018).

Myasthenia gravis (MG) is a T cell-dependent, B-cell mediated chronic autoimmune disease caused by antibodies against the AChR, MuSK, or low-density LRP4 expressed in postsynaptic muscle cells, which manifests in muscle weakness and fatigue. About 80% of patients with MG show anti-AChR antibody positivity, and about 40% of the anti-AChR antibody-negative patients show anti-MuSK antibody positivity. The presence of anti-LRP4 autoantibodies can be detected among patients outside the previous groups (Müzes and Sipos Cells (2023) 12 (11): 1534). Conventional treatment options, including symptomatic treatments and general immunosuppression, can help, though durable remission remains improbable, and chronic treatment with high doses of non-specific immunosuppressive drugs is usually necessary to maintain disease remission (Wu 2020). More recent treatment approaches include B cell-targeting therapies such as monoclonal antibodies and proteasome-targeting inhibitors. However, there remains an unmet need for effective treatments, particularly for patients with refractory disease (Huda, Front. Immunol. (2020) 11:240).

Vasculitis is classified primarily by the predominant size of the vessels involved, and the 2012 Chapel Hill Consensus Conference (CHCC) represents a widely used nomenclature and classification system for vasculitis (Jennette et al., Arthritis Rheum. (2013) 65:1-11). Specifically, vasculitis is subdivided into large-vessel, medium-vessel, and small-vessel vasculitis. Large-vessel vasculitis includes Takayasu arteritis (TAK) and giant cell arteritis (GCA), which primarily affect the aorta and its major branches. Medium-vessel vasculitis includes polyarteritis nodosa (PAN) and Kawasaki disease, which typically affect medium- and small-sized arteries. Small-vessel vasculitis includes ANCA-associated vasculitis (AAV), which is a systemic autoimmune disease that affects small sized blood vessels and can lead to serious complications in the lungs and kidneys (Merino-Vico 2022). AAV encompasses three major types of vasculitides that have different clinical characteristics, namely granulomatosis with polyangiitis (GPA, previously referred to as Wegener's granulomatosis), microscopic polyangiitis (MPA), and eosinophilic granulomatosis with polyangiitis (EGPA) (Jennette and Falk, Semin. Immunopathol. (2014) 36:327-38). The prominent presence of ANCA autoantibodies in this disease implicates B cells in its pathogenesis, as these are the precursors of the ANCA-producing plasma cells (PCs). Further evidence supporting the potential role of B lineage cells in vasculitis are the increased B cell cytokine levels and the dysregulated B cell populations in patients. Confirmation of the contribution of B cells to pathology arose from the beneficial effect of anti-CD20 therapy (i.e., rituximab) in AAV patients. These anti-CD20 antibodies deplete circulating B cells, which results in amelioration of disease. However, not all patients respond completely, and this treatment does not target PCs, which can maintain ANCA production (Merino-Vico 2022).

More recently, anti-CD19 autologous CAR T cells have been investigated for treatment of autoimmune diseases such as SLE (Muller et al., N Engl J Med (2024) 390 (8): 687-700), MG (NCT05828225), myositis (Pecher et al., JAMA (2023) 329 (24): 2154-62), antisynthetase syndrome (Müller et al., Lancet (2023) 401 (10379): 815-18), systemic scleroderma (Bergmann et al., Ann Rheum Dis. (2023) 82 (8): 1117-20), and vasculitis (NCT05263817). Autologous CD19 CAR T cell treatment achieved sustained depletion of circulating B cells, disappearance of serum autoantibodies, and clinical remission in a patient with severe refractory SLE who was nonresponsive to anti-BAFF and anti-CD20 antibody treatments (Mougiakakos et al., N Engl J Med (2021) 385:567-59). Following administration to the patient, the CD19 CAR T cells were observed to rapidly expand and be detectable for the following seven weeks, with expansion of the CAR T cells preceding the complete and sustained depletion of circulating B cells. See Mougiakaos et al. (2021); and Jin et al., Cell Mol Immunol (2021) 18:2581-82. Long-term follow-up with additional patients found that B cells reappeared after a mean of 110 days following CAR T cell treatment (Mackensen et al., Nat Med (2022) 28 (10): 2124-32), with B cell aplasia lasting for a median of 120 days (Taubmann et al. (2023) Ann Rheum Dis 82 (Suppl. 1): 93-4).

Despite this, autologous CAR T cells have been historically plagued by concerns about associated toxicities such as cytokine release syndrome (CRS) and immune effector cell-associated neurotoxicity syndrome (ICANS). Autologous cell therapies are also manufactured after cell collection from subjects and are not ready for use until weeks later, and occasionally fail to expand. Further, as autologous CAR T cells are derived from subjects who are not healthy, it is generally the case that a subject cannot undergo retreatment (including an additional round of lymphodepletion and leukapheresis) in the event of a manufacturing or treatment failure or disease relapse. The limited ability to retreat patients with autologous CAR T cells may be particularly relevant in the context of autoimmune diseases, which tend to be chronic in nature and require treatment, or retreatment, over long periods of time. In addition, while CD19 CAR T cells were observed to produce complete and sustained depletion of circulating B cells in a subject with SLE, the total elimination of B cells (including all subpopulations thereof) may increase a patient's risk of infection.

Unlike T cells, allogeneic NK cells are not observed to induce graft vs. host disease (GvHD) and can kill cells in an antigen-independent manner. Further, allogeneic cell therapies (e.g., an allogeneic NK cell therapy) are derived from healthy donors, such that patients do not have to undergo leukapheresis. Allogeneic therapies can therefore be provided on-demand. An allogeneic CAR NK cell therapy may therefore provide additional opportunities for retreatment of a subject with a chronic autoimmune disease, as allogeneic cells can be produced from healthy subjects as needed and present a lower risk of toxicities such as CRS and ICANS. For example, in some aspects, if a subject exhibits an initial clinical benefit (e.g., PR or CR), an additional dosing cycle is provided to consolidate or deepen the response. In some aspects, if a subject exhibits a CR, an additional dosing cycle is provided to consolidate the response. In some aspects, if a subject exhibits a PR, an additional dosing cycle is provided to deepen the response. In some aspects, if a subject exhibits an initial clinical benefit (e.g., PR or CR) and subsequent disease progression, an additional dosing cycle is provided as retreatment.

While the in vivo persistence of CAR NK cells has been observed to be shorter than that of CAR T cells, it is contemplated that this quality of CAR NK cells may allow for reduced long-term B cell suppression and reduced risk of infection. Indeed, findings described herein show that treatment with NK cells expressing a CD19 CAR achieve deep suppression of CD19+ B cells within about 30 days, with B cell counts recovering significantly within a few months of the last dose. Further, data in the Working Examples reveal that B cell recovery after CD19 CAR NK treatment results in a predominantly naïve (e.g., non-class-switched) B cell population. Without wishing to be bound by theory, and as class switching to mature B cell isotypes appears to be required for the generation of pathogenic autoreactive antibodies in SLE (Liu et al., Autoimmunity (2004) 37 (6-7): 431-43), the data suggest that treatment with NK cells expressing a CD19 CAR could reset the B cell compartment, thereby reducing or eliminating the production of autoreactive antibodies by mature B cells. Thus, alternative treatments are needed that provide improved persistence, potency, and/or availability over current strategies, while minimizing potential toxicities and side effects.

Further, it is contemplated herein that the relatively limited persistence of CAR NK cells in vivo may allow for modification of the standard lymphodepletion regimens used with CAR T cell therapies, such that the risk of potential toxicities can be mitigated. Multiple studies have shown that adequate suppression of the host immune system correlates with responses in cell therapy clinical trials (Miller et al., Blood (2005) 105 (8): 3051-57; Turtle et al., J. Clin. Oncol. (2017) 35 (26): 3010-20). Thus, lymphodepleting therapy has been an integral part of CAR T cell clinical trials. Similarly, use of high-dose lymphodepletion regimens prior to adoptive transfer of NK cell therapies has yielded in vivo NK cell expansion and persistence, whereas low-dose lymphodepletion regimens did not (Kilgour et al., Front. Immunol. (2023) 14:1166038). In particular, a lymphodepletion regimen of fludarabine and cyclophosphamide has historically been associated with the ability to detect adoptively transferred immune cells. The benefit of eliminating lymphocytes via a fludarabine-containing lymphodepleting therapy may be realized not just through reduced rejection of adoptively transferred immune cells, but also through improved availability of cytokines such as interleukin 15 (IL15) (Gauthier et al., Blood (2020) 136 (Supp. 1): 37-38).

However, CD19 CAR-expressing NK cells as provided herein are expected to have much of their activity shortly after administration to a subject, such that the primary benefit of lymphodepletion for NK cells may be from cyclophosphamide, which has activity against lymphocytes earlier than fludarabine. For example, the nadir of white blood cell counts has been reported to be approximately 13 days after fludarabine treatment (FLUDARA® USPI 2010) compared to approximately 9 days after cyclophosphamide treatment (Buckner et al., Cancer (1972) 29 (2): 357-65). In connection with this, where multiple doses of CD19 CAR-expressing NK cells are provided in a single dosing cycle, the provision of all doses within the peak activity window of cyclophosphamide (e.g., within about 7-10 days of the administration of cyclophosphamide) may be particularly advantageous. The inventors therefore contemplate the combination of a cyclophosphamide-only lymphodepleting therapy in combination with a dosing cycle in which all doses of CD19 CAR-expressing NK cells are provided within about 7-10 days of administration of the cyclophosphamide-only lymphodepleting therapy. For example, a single dose of cyclophosphamide can be provided about 3 days prior to administration of the first dose of CD19 CAR NK cells (Day −3), and the doses of CD19 CAR NK cells can be provided on Days 0, 3, and 7. Similarly, a single dose of cyclophosphamide can be provided about 3 days prior to administration of the first dose of CD19 CAR NK cells (Day −3), and the doses of CD19 CAR NK cells can be provided on Days 0, 2, and 4 or on Days 0, 2, and 5. To this end, the inventors contemplate that a dosing cycle comprising administration of a single dose of cyclophosphamide on Day-3 and administration of the CD19 CAR NK cells on Days 0, 2, and 4 could be particularly convenient in an outpatient setting, where cyclophosphamide could be provided e.g., on Friday, and doses of CD19 CAR NK cells could be provided e.g., on the following Monday, Wednesday, and Friday. Without wishing to be bound by theory, the provision of each of the doses of CD19 CAR-expressing NK cells within about 7-10 days of the administration of cyclophosphamide may allow for improved peak concentration and/or persistence of the NK cells as compared to a dosing regimen in which one or more doses of the dosing cycle are provided later in time.

Further, increased cytokine (e.g., IL15) bioavailability may be unnecessary with CD19 CAR-expressing NK cells expressing membrane-bound interleukin 15 (mbIL15), including those as provided herein. Similar considerations could apply to NK cells genetically engineered to increase IL15 signaling (e.g., via knockout of the CISH gene). As fludarabine may increase not only short-term toxicity (Hay et al., Blood (2017) 130 (21): 2295-2306) but also the potential for secondary malignancies, removing fludarabine from lymphodepleting therapy may improve the risk-benefit profile. Thus, it is contemplated that use of the CD19 CAR-expressing NK cells as provided herein to treat or prevent an autoimmune disease may not require a fludarabine-containing lymphodepletion regimen, as is commonly used for hematologic malignancies. Rather, a lymphodepletion regimen of only cyclophosphamide may be sufficient to achieve efficacy and reduce potential toxicities associated with LD.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Cell Therapy and Engineering Cells

In some embodiments, the composition (e.g., genetically engineered NK cell composition) for use in accord with the provided methods includes administering engineered NK cells expressing recombinant receptors (e.g. CAR) designed to recognize and/or specifically bind to an antigen associated with the autoimmune disease. In particular embodiments, the antigen that is bound or recognized by the recombinant receptor (e.g. CAR) is CD19. In some embodiments, binding to the antigen results in a response, such as an immune response against such antigen. In some embodiments, binding to the antigen results in the reduction or depletion of cells expressing the antigen (e.g., B cells expressing CD19, or a subset thereof). For example, binding to the antigen may reduce or deplete peripheral B cells in a subject being treated. The reduction or depletion of B cells may correspondingly reduce the level and/or activity of an autoantibody in the subject.

In some embodiments, the genetically engineered cells contain or are engineered to contain the recombinant receptor, such as a chimeric antigen receptor (CAR). The recombinant receptor, such as a CAR, generally includes an extracellular antigen-binding domain specific to the antigen (e.g., CD19), which is linked to one or more intracellular signaling components, in some aspects via linkers and/or transmembrane domain(s). In some aspects, the genetically engineered NK cells are provided as pharmaceutical compositions and formulations suitable for administration to a subjects, such as for cell therapy. Also provided are therapeutic methods for administering the cells and compositions to subjects, uses of the compositions for treatment of subjects, and uses of the compositions in the manufacture of medicaments for treating subjects.

### A. Chimeric Antigen Receptors

Among the provided recombinant receptors, e.g., CD19-directed CARs, are chimeric receptors that specifically bind to CD19, such as receptors comprising an anti-CD19 antibody, e.g., antibody fragment. Among the antigen receptors are chimeric antigen receptors (CARs). Also provided are immune cells (e.g., NK cells) expressing the recombinant receptors and uses thereof in treatment of diseases and condition, such as autoimmune diseases (e.g., SLE). The chimeric receptors, such as CARs, generally include an extracellular antigen-binding domain that includes an anti-CD19 antibody. Such recombinant receptors include antibodies (including antigen-binding fragments thereof) that specifically bind to CD19 proteins, such as human CD19 protein (e.g., SEQ ID NO:39). In some embodiments, the antibodies include those that are multi-domain antibodies, such as those containing V_{H} and V_{L} domains. In some embodiments, the antibodies include a variable heavy chain and a variable light chain, such as scFvs. Among the provided anti-CD19 antibodies are human and humanized antibodies.

The term “antibody” herein is used in the broadest sense and includes polyclonal and monoclonal antibodies, including intact antibodies and functional (antigen-binding) antibody fragments, including fragment antigen binding (Fab) fragments. F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibody (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM. IgE, IgA, and IgD.

The terms “complementarity determining region.” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including the Kabat numbering scheme (Sequences of Proteins of Immunological Interest, 1987 and 1991, NIH, Bethesda, MD), the Chothia numbering scheme (Chothia & Lesk, 1987, J. Mol. Biol. 196:901-917; Chothia et al., 1989, Nature 342:878-883), the Contact numbering scheme (MacCallum et al., J. Mol. Biol. 262:732-745 (1996), “Antibody-antigen interactions: Contact analysis and binding site topography,” J. Mol. Biol. 262, 732-745), the AbM numbering scheme (Martin et al., Proc. Natl. Acad. Sci., 86:9268-9272; 1989), the IMGT numbering scheme (the international ImMunoGeneTics information system; Lefranc et al, Dev. Comp. Immunol. 29:185-203; 2005), and the Aho numbering scheme (Honegger and Pluckthun, J. Mol. Biol. 309 (3): 657-670; 2001).

The boundaries of a given CDR or FR may vary depending on the scheme used for identification. For example, the Kabat scheme is based structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Table 1, below, lists non-limiting position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located between CDR-L1 and CDR-L2, and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes

| CDR | Kabat | Chothia | AbM | Contact |
|---|---|---|---|---|
| CDR-L1 | L24 - - L34 | L24 - - L34 | L24 - - L34 | L30 - - L36 |
| CDR-L2 | L50 - - L56 | L50 - - L56 | L50 - - L56 | L46 - - L55 |
| CDR-L3 | L89 - - L97 | L89 - - L97 | L89 - - L97 | L89 - - L96 |
| CDR-H1 | H31 - - H35B | H26 - - H32.34 | H26 - - H35B | H30 - - H35B |
| (Kabat numbering¹) | | | | |
| CDR-H1 | H31 - - H35 | H26 - - H32 | H26 - - H35 | H30 - - H35 |
| (Chothia numbering²) | | | | |
| CDR-H2 | H50 - - H65 | H52 - - H56 | H50 - - H58 | H47 - - H58 |
| CDR-H3 | H95 - - H102 | H95 - - H102 | H95 - - H102 | H93 - - H101 |

¹Kabat et al., “Sequences of Proteins of Immunological Interest,” (1991) 5th Ed. Public Health Service, National Institutes of Health, Bethesda, MD.
²Al-Lazikani et al., J. Mol. Biol. (1997) 273(4): 927-48.

Thus, unless otherwise specified, a “CDR” or “complementary determining region,” or individual specified CDRs (e.g., “CDR-H1, CDR-H2), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given V_{H} or V_{L} amino acid sequence, it is understood that such a CDR has a sequence of the corresponding CDR (e.g., CDR-H3) within the variable region, as defined by any of the aforementioned schemes. In some embodiments, specific CDR sequences are specified.

Likewise, unless otherwise specified, a FR or individual specified FR(s) (e.g., FR-H1, FR-H2), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the IMGT, Kabat, Chothia, or Contact method. In other cases, the particular amino acid sequence of a CDR or FR is given.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. A single V_{H} or V_{L} domain may be sufficient to confer antigen-binding specificity.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and a variable light chain region, such as scFvs.

In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

Single-domain antibodies are antibody fragments comprising all or a portion of the heavy chain variable domain or all or a portion of the light chain variable domain of an antibody. In certain embodiments, a single-domain antibody is a human or humanized single-domain antibody.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided anti-CD19 antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

The terms “polypeptide” and “protein” are used interchangeably to refer to a polymer of amino acid residues and are not limited to a minimum length. Polypeptides, including the provided antibodies and antibody chains and other peptides, e.g., linkers and CD19-binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity.

The antigen-binding domain may be or comprise any antibody (e.g., anti-CD19 antibody) as described herein.

In some embodiments, the extracellular antigen-binding domain comprises a heavy chain variable region (VH) having a CDR-1, a CDR-2, and a CDR-3 comprising the amino acid sequences set forth in SEQ ID NOS: 24, 25, and 26, respectively. In some embodiments, the extracellular antigen-binding domain comprises a light chain variable region (VL) having a CDR-1, a CDR-2, and a CDR-3 comprising the amino acid sequences set forth in SEQ ID NOS: 27, 28, and 29, respectively. In some embodiments, the extracellular antigen-binding domain comprises a VH having a CDR-1, a CDR-2, and a CDR-3 comprising the amino acid sequences set forth in SEQ ID NOS: 24, 25, and 26, respectively; and a VL having a CDR-1, a CDR-2, and a CDR-3 comprising the amino acid sequences set forth in SEQ ID NOS: 27, 28, and 29, respectively.

In some embodiments, the extracellular antigen-binding domain comprises a heavy chain variable region (VH) having a CDR-1, a CDR-2, and a CDR-3 comprising the amino acid sequences set forth in SEQ ID NOS: 30, 31, 32, respectively. In some embodiments, the extracellular antigen-binding domain comprises a light chain variable region (VL) having a CDR-1, a CDR-2, and a CDR-3 comprising the amino acid sequences set forth in SEQ ID NOS: 33, 34, and 29, respectively. In some embodiments, the extracellular antigen-binding domain comprises a VH having a CDR-1, a CDR-2, and a CDR-3 comprising the amino acid sequences set forth in SEQ ID NOS: 30, 31, and 32, respectively; and a VL having a CDR-1, a CDR-2, and a CDR-3 comprising the amino acid sequences set forth in SEQ ID NOS: 33, 34, and 29, respectively.

In some embodiments, the VH comprises an amino acid sequence having at least about 80%, 85%, 90%, 91%, 92%, 93%, 94%, 95%, 96%, 97%, 98%, 99%, or more sequence identity to the amino acid sequence set forth in SEQ ID NO:35. In some embodiments, the VH comprises the amino acid sequence set forth in SEQ ID NO:35. In some embodiments, the VL comprises an amino acid sequence having at least about 80%, 85%, 90%, 91%, 92%, 93%, 94%, 95%, 96%, 97%, 98%, 99%, or more sequence identity to the amino acid sequence set forth in SEQ ID NO: 36. In some embodiments, the VL comprises the amino acid sequence set forth in SEQ ID NO: 36. In some embodiments, the VH comprises an amino acid sequence having at least about 80%, 85%, 90%, 91%, 92%, 93%, 94%, 95%, 96%, 97%, 98%, 99%, or more sequence identity to the amino acid sequence set forth in SEQ ID NO:35; and the VL comprises an amino acid sequence having at least about 80%, 85%, 90%, 91%, 92%, 93%, 94%, 95%, 96%, 97%, 98%, 99%, or more sequence identity to the amino acid sequence set forth in SEQ ID NO:36. In some embodiments, the VH comprises the amino acid sequence set forth in SEQ ID NO:35, and the VL comprises the amino acid sequence set forth in SEQ ID NO:36.

In some embodiments, the antigen-binding domain is an scFv comprising a VH and a VL joined by a linker (e.g., a linker comprising any of SEQ ID NOS: 1-3). In some embodiments the linker comprises the amino acid sequence as set forth in SEQ ID NO:1 or SEQ ID NO: 3. In some embodiments, the extracellular antigen-binding domain is an scFv comprising the linker set forth in SEQ ID NO: 1. In some embodiments, the antigen-binding domain is an scFv comprising an amino acid sequence having at least about 80%, 85%, 90%, 91%, 92%, 93%, 94%, 95%, 96%, 97%, 98%, 99%, or more sequence identity to the amino acid sequence set forth in SEQ ID NO: 37. In some embodiments, the antigen-binding domain is an scFv comprising the amino acid sequence set forth in SEQ ID NO:37. In some embodiments, the extracellular antigen-binding domain is an scFv comprising the linker set forth in SEQ ID NO:2. In some embodiments, the extracellular antigen-binding domain is an scFv comprising the linker set forth in SEQ ID NO:3.

Additional CD19-binding domains are known and described in the art, including any of those as described in PCT Application Nos. PCT/US2015/024671, PCT/US2018/029107, PCT/US2020/020824, PCT/US2020/033559, PCT/IB2021/060213, and PCT/CN2021/106892, each of which is incorporated herein in its entirety by reference.

Provided herein are recombinant receptors (e.g., CARs) comprising any of the CD19 antibodies or binding domains described herein. The extracellular antigen-binding domain generally is linked to an intracellular signaling domain comprising intracellular signaling components, such as signaling components that mimic activation through an antigen receptor complex, such as a TCR complex, in the case of a CAR. In some embodiments, the extracellular antigen-binding domain of a CAR is linked to an intracellular signaling domain by a transmembrane domain. Thus, in some embodiments, the CD19-binding molecule (e.g., antibody) is linked to a transmembrane and intracellular signaling domain. In some embodiments, a CAR comprises an extracellular antigen-binding domain that binds to CD19, a transmembrane domain, and an intracellular signaling domain comprising a co-stimulatory signal region and a primary signaling domain (e.g., CD3zeta).

In some embodiments, the transmembrane domain is fused to the extracellular domain. The transmembrane domain in some embodiments is derived either from a natural or from a synthetic source. Where the source is natural, the domain in some aspects is derived from any membrane-bound or transmembrane protein. Transmembrane regions include those derived from (e.g., comprising at least the transmembrane region(s) of) CD3, CD4, CD5, CD8, CD9, CD 16, CD22, CD28, CD33, CD37, CD45, CD64, CD80, CD86, CD134, CD137, CD154, or a combination thereof. Alternatively, the transmembrane domain in some embodiments is synthetic.

In several embodiments, the transmembrane domain comprises at least a portion of CD8, a transmembrane glycoprotein normally expressed on both T cells and NK cells. In several embodiments, the transmembrane domain comprises CD8alpha (CD8a). In several embodiments, the transmembrane domain comprises a CD8 (e.g., CD8a) hinge and a CD8 (e.g., CD8a) transmembrane region.

In several embodiments, the transmembrane domain comprises a hinge, e.g. a CD8a hinge. In several embodiments, the sequence encoding the CD8a hinge is truncated or modified. In some embodiments, the CD8a hinge is encoded by a nucleic acid sequence that has at least 70%, at least 75%, at least 80%, at least 85%, at least 90%, at least 95% sequence identity to the nucleic acid sequence of SEQ ID NO:5. In several embodiments, the CD8a hinge comprises the nucleic acid sequence of SEQ ID NO:5. In several embodiments, the CD8a hinge is truncated or modified. In some embodiments, the CD8a hinge has at least 70%, at least 75%, at least 80%, at least 85%, at least 90%, at least 95% sequence identity to the amino acid sequence of SEQ ID NO:6. In several embodiments, the hinge of CD8a comprises the amino acid sequence of SEQ ID NO:6.

In several embodiments, the transmembrane domain comprises a CD8a transmembrane region. In several embodiments, the CD8a transmembrane region is truncated or modified. In some embodiments, the CD8a transmembrane region is encoded by a nucleic acid sequence that has at least 70%, at least 75%, at least 80%, at least 85%, at least 90%, at least 95% sequence identity to the sequence of SEQ ID NO:7. In several embodiments, the CD8a transmembrane region is encoded by a nucleic acid sequence of SEQ ID NO:7. In several embodiments, the CD8a transmembrane region is truncated or modified. In some embodiments, the CD8a transmembrane region has at least 70%, at least 75%, at least 80%, at least 85%, at least 90%, at least 95% sequence identity to the sequence of SEQ ID NO:8. In several embodiments, the CD8a transmembrane region comprises the amino acid sequence of SEQ ID NO:8.

Thus, in several embodiments, the CD8 transmembrane domain is truncated or modified and is encoded by a nucleic acid sequence that has at least 70%, at least 75%, at least 80%, at least 85%, at least 90%, at least 95% sequence identity to the sequence of SEQ ID NO:9. In several embodiments, the CD8 transmembrane domain is encoded by the nucleic acid sequence of SEQ ID NO:9. In some embodiments, the CD8 transmembrane domain is truncated or modified and comprises an amino acid sequence that has at least 70%, at least 75%, at least 80%, at least 85%, at least 90%, at least 95% sequence identity to the sequence of SEQ ID NO:10. In several embodiments, the transmembrane domain comprises the amino acid sequence of SEQ ID NO:10.

In some embodiments, the transmembrane domain comprises a CD28 transmembrane domain or a fragment thereof. In several embodiments, the CD28 transmembrane domain is truncated or modified. In some embodiments, the CD28 transmembrane domain has at least 70%, at least 75%, at least 80%, at least 85%, at least 90%, at least 95% sequence identity to the amino acid sequence of SEQ ID NO:11. In several embodiments, the CD28 transmembrane domain comprises the amino acid sequence of SEQ ID NO: 11.

The receptor, e.g., the CAR, generally includes an intracellular signaling domain comprising intracellular signaling components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, upon ligation of the CAR or other chimeric receptor, the cytoplasmic domain or intracellular signaling domain of the receptor activates at least one of the normal effector functions or responses of the immune cell, e.g., NK cell engineered to express the CAR. For example, in some contexts, the CAR induces a function of an immune cell (e.g., NK cell) such as cytolytic activity and/or secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain includes the cytoplasmic sequences of the T cell receptor (TCR), and in some aspects, also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the receptor. T cell activation is in some aspects described as being mediated by two classes of cytoplasmic signaling sequences: those that initiate antigen-dependent primary activation through the TCR (primary cytoplasmic signaling sequences), and those that act in an antigen independent manner to provide a secondary or co-stimulatory signal (secondary cytoplasmic signaling sequences). In some aspects, the receptor includes one or both of such signaling components.

In some aspects, the receptor includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3zeta.

For example, immune cells engineered according to several embodiments disclosed herein may comprise at least one subunit of the CD3 T cell receptor complex (or a fragment thereof). In several embodiments, the signaling domain comprises the CD3 zeta subunit. In several embodiments, the CD3zeta can be truncated or modified. In some embodiments, the CD3zeta is encoded by a nucleic acid sequence that has at least 70%, at least 75%, at least 80%, at least 85%, at least 90%, at least 95% sequence identity to the nucleic acid sequence of SEQ ID NO: 17. In several embodiments, the CD3zeta is encoded by the nucleic acid sequence of SEQ ID NO: 17. In several embodiments, the CD3zeta is truncated or modified. In some embodiments, the CD3zeta comprises an amino acid sequence that has at least 70%, at least 75%, at least 80%, at least 85%, at least 90%, at least 95% sequence identity to the amino acid sequence of SEQ ID NO: 18. In several embodiments, the CD3zeta comprises the amino acid sequence of SEQ ID NO: 18.

In some embodiments, the intracellular signaling domain comprises a costimulatory signaling region, such as an intracellular signaling region of CD28, 4-1BB, OX40, DAP10, ICOS, or any combination thereof. In some embodiments, the intracellular signaling domain comprises an intracellular signaling region of CD28. In some embodiments, the intracellular signaling domain comprises a an intracellular signaling region of 4-1BB. In some embodiments, the intracellular signaling domain comprises a an intracellular signaling region of OX40. In some embodiments, the intracellular signaling domain comprises a an intracellular signaling region of DAP10. In some embodiments, the intracellular signaling domain comprises a an intracellular signaling region of ICOS. In some embodiments, the intracellular signaling domain does not include DAP10 and/or DAP12. In some embodiments, the intracellular signaling domain does not include DAP10. In some embodiments, the intracellular signaling domain does not include DAP12. In some aspects, the same receptor includes both a CD3zeta and a costimulatory signaling region. Thus, in some embodiments, the intracellular signaling domain of the recombinant receptor, such as CAR, comprises a CD3zeta intracellular domain and a costimulatory signaling region.

In several embodiments, the intracellular signaling domain comprises an intracellular signaling region of OX40. In several embodiments, the OX40 intracellular signaling region is encoded by a nucleic acid sequence that has at least 70%, at least 75%, at least 80%, at least 85%, at least 90%, at least 95% sequence identity to the nucleic acid sequence of SEQ ID NO: 13. In several embodiments, the OX40 intracellular signaling region is encoded by the nucleic acid sequence of SEQ ID NO:13. In several embodiments, the OX40 intracellular signaling region comprises an amino acid sequence that has at least 70%, at least 75%, at least 80%, at least 85%, at least 90%, at least 95% sequence identity to the amino acid sequence of SEQ ID NO: 14. In several embodiments, the OX40 intracellular signaling region comprises the amino acid sequence of SEQ ID NO: 14. In several embodiments, OX40 is used as the sole intracellular signaling component in the construct, however, in several embodiments, OX40 can be used with one or more other components. For example, combinations of OX40 and CD3zeta are used in some embodiments. In some embodiments, the intracellular signaling domain comprises an OX40 costimulatory signaling region linked to CD3zeta.

In some embodiments, the CAR comprises an extracellular antigen-binding domain comprising the sequence set forth in SEQ ID NO:37, a CD8alpha transmembrane domain comprising the amino acid sequence set forth in SEQ ID NO:8, an OX40 intracellular signaling region comprising the amino acid sequence set forth in SEQ ID NO:14, and a CD3zeta domain comprising the amino acid sequence set forth in SEQ ID NO:18. In some embodiments, the CAR comprises an amino acid sequence having at least about 80%, 85%, 90%, 91%, 92%, 93%, 94%, 95%, 96%, 97%, 98%, 99%, or more sequence identity to the amino acid sequence set forth in SEQ ID NO: 38. In some embodiments, the CAR comprises the amino acid sequence set forth in SEQ ID NO: 38.

By way of further example, combinations of CD28, OX40, 4-1BB and/or CD3zeta are used in some embodiments.

In several embodiments, the intracellular signaling domain comprises an intracellular signaling region of 4-1BB. In several embodiments, the 4-1BB intracellular signaling region is encoded by a nucleic acid sequence that has at least 70%, at least 75%, at least 80%, at least 85%, at least 90%, at least 95% sequence identity to the nucleic acid sequence of SEQ ID NO: 15. In several embodiments, the 4-1BB intracellular signaling region is encoded by the nucleic acid sequence of SEQ ID NO:15. In several embodiments, the 4-1BB intracellular signaling region comprises an amino acid sequence that has at least 70%, at least 75%, at least 80%, at least 85%, at least 90%, at least 95% sequence identity to the amino acid sequence of SEQ ID NO:16. In several embodiments, the 4-1BB intracellular signaling region comprises the amino acid sequence of SEQ ID NO: 16. In several embodiments, 4-1BB is used as the sole intracellular signaling component in the construct, however, in several embodiments, 4-1BB can be used with one or more other components. For example, combinations of 4-1BB and CD3zeta are used in some embodiments. In some embodiments, the intracellular signaling domain comprises a 4-1BB costimulatory signaling region linked to CD3zeta. By way of further example, combinations of CD28, OX40, 4-1BB and/or CD3zeta are used in some embodiments.

In several embodiments, the intracellular signaling domain comprises an intracellular signaling region of CD28. In several embodiments, the CD28 intracellular signaling region comprises an amino acid sequence that has at least 70%, at least 75%, at least 80%, at least 85%, at least 90%, at least 95% sequence identity to the amino acid sequence of SEQ ID NO: 12. In several embodiments, the CD28 intracellular signaling region comprises the amino acid sequence of SEQ ID NO:12. In several embodiments, CD28 is used as the sole intracellular signaling component in the construct, however, in several embodiments, CD28 can be used with one or more other components. For example, combinations of CD28 and CD3zeta are used in some embodiments. In some embodiments, the intracellular signaling domain comprises a CD28 costimulatory signaling region linked to CD3zeta. By way of further example, combinations of CD28, OX40, 4-1BB and/or CD3zeta are used in some embodiments.

Additional CD19-directed CARs are known and described in the art, including any of those as described in Kalos et al., Sci Transl Med 3: 95ra73 (2011); Porter et al., NEJM 365:725-733 (2011); Grupp et al., NEJM 368:1509-1518 (2013); and PCT Application Nos. PCT/US2015/024671. PCT/US2018/029107. PCT/US2020/020824, and PCT/CN2021/106892.

In any of the provided embodiments, the nucleic acid encoding the chimeric receptor, or a portion thereof, is codon-optimized. In some embodiments, the polynucleotides are optimized, or contain certain features designed for optimization, such as for codon usage, to reduce RNA heterogeneity and/or to modify, e.g., increase or render more consistent among cell product lots, expression, such as surface expression, of the encoded receptor. In some embodiments, polynucleotides, encoding chimeric receptors, are modified as compared to a reference polynucleotide, such as to remove cryptic or hidden splice sites, to reduce RNA heterogeneity. In some embodiments, polynucleotides, encoding chimeric receptors, are codon optimized, such as for expression in a mammalian, e.g., human, cell such as in a human T cell. In some aspects, the modified polynucleotides result in improved, e.g., increased or more uniform or more consistent level of, expression, e.g., surface expression, when expressed in a cell.

### B. Engineered Cells

Also provided are methods, nucleic acids, compositions, and kits, for producing the genetically engineered immune cells (e.g., NK cells). In some aspects, the genetic engineering involves introduction of a nucleic acid encoding the genetically engineered component or other component for introduction into the cell, such as a component encoding a gene-disrupting protein or nucleic acid. Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo.

i. Vectors and Methods for Genetic Engineering

Also provided are methods, polynucleotides, compositions, and kits, for expressing the binding molecules (e.g., anti-CD19 binding molecules), including recombinant receptors (e.g., CARs) comprising the binding molecules, and for producing the genetically engineered immune cells (e.g., NK cells) expressing such binding molecules. In some embodiments, one or more binding molecules, including recombinant receptors (e.g., CARs) can be genetically engineered into cells or a plurality of cells. The genetic engineering generally involves introduction of a nucleic acid encoding the recombinant or engineered component into the cell, such as by retroviral transduction, transfection, or transformation.

Also provided are polynucleotides encoding the antibodies and chimeric antigen receptors and/or portions, e.g., chains, thereof. Among the provided polynucleotides are those encoding the anti-CD19 chimeric antigen receptors (e.g., antigen-binding fragment) described herein. Also provided are polynucleotides encoding one or more antibodies and/or portions thereof. e.g., those encoding one or more of the anti-CD19 antibodies (e.g., antigen-binding fragment) described herein and/or other antibodies and/or portions thereof, e.g., antibodies and/or portions thereof that binds other target antigens. The polynucleotides may include those encompassing natural and/or non-naturally occurring nucleotides and bases, e.g., including those with backbone modifications. The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide. Also provided are polynucleotides that have been optimized for codon usage.

Also provided are vectors containing the polynucleotides, such as any of the polynucleotides described herein, and cells containing the vectors, e.g., for producing the antibodies or antigen-binding fragments thereof. In some embodiments, the vector is a viral vector. In some embodiments, the vector is a retroviral vector. In some embodiments, the vector is a lentiviral vector. Also provided are methods for producing the antibodies or antigen-binding fragments thereof. The nucleic acid may encode an amino acid sequence comprising the VL region and/or an amino acid sequence comprising the VH region of the antibody (e.g., the light and/or heavy chains of the antibody). The nucleic acid may encode one or more amino acid sequence comprising the VL region and/or an amino acid sequence comprising the VH region of the antibody (e.g., the light and/or heavy chains of the antibody). In a further embodiment, one or more vectors (e.g., expression vectors) comprising such polynucleotides are provided. In a further embodiment, a host cell comprising such polynucleotides is provided. In another such embodiment, a host cell comprises (e.g., has been transformed with) (1) a vector comprising a nucleic acid that encodes an amino acid sequence comprising the VL region of the antibody and an amino acid sequence comprising the VH region of the antibody, or (2) a first vector comprising a nucleic acid that encodes an amino acid sequence comprising the VL region of the antibody and a second vector comprising a nucleic acid that encodes an amino acid sequence comprising the VH region of the antibody. In some embodiments, a host cell comprises (e.g., has been transformed with) one or more vectors comprising one or more nucleic acid that encodes one or more an amino acid sequence comprising one or more antibodies and/or portions thereof, e.g., antigen-binding fragments thereof. In some embodiments, one or more such host cells are provided. In some embodiments, a composition containing one or more such host cells are provided. In some embodiments, the one or more host cells can express different antibodies, or the same antibody. In some embodiments, each of the host cells can express more than one antibody.

Also provided are methods of making the anti-CD19 chimeric antigen receptors. For recombinant production of the chimeric receptors, a nucleic acid sequence encoding a chimeric receptor antibody, e.g., as described herein, may be isolated and inserted into one or more vectors for further cloning and/or expression in a host cell. Such nucleic acid sequences may be readily isolated and sequenced using conventional procedures (e.g., by using oligonucleotide probes that are capable of binding specifically to genes encoding the heavy and light chains of the antibody). In some embodiments, a method of making the anti-CD19 chimeric antigen receptor is provided, wherein the method comprises culturing a host cell comprising a nucleic acid sequence encoding the antibody, as provided above, under conditions suitable for expression of the receptor. In particular examples, immune cells, such as human immune cells are used to express the provided polypeptides encoding chimeric antigen receptors. In some examples, the immune cells are NK cells including primary NK cells.

In some embodiments, gene transfer is accomplished by transduction of the immune cells (e.g., activated immune cells), and expansion in culture to numbers sufficient for clinical applications. In some aspects, the cells further are engineered to promote expression of cytokines or other factors. Various methods for the introduction of genetically engineered components, e.g., antigen receptors, e.g., CARs, are well known and may be used with the provided methods and compositions. Non-limiting examples of methods include those for transfer of polynucleotides encoding the receptors, including via viral, e.g., retroviral or lentiviral, transduction, transposons, and electroporation.

In some embodiments, recombinant polynucleotides are transferred into immune cells (e.g., NK cells) using recombinant infectious virus particles, such as, e.g., vectors derived from simian virus 40 (SV40), adenoviruses, adeno-associated virus (AAV). In some embodiments, recombinant polynucleotides are transferred into immune cells using recombinant lentiviral vectors or retroviral vectors, such as gamma-retroviral vectors. In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or human immunodeficiency virus type 1 (HIV-1). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described. Methods of lentiviral transduction are known and described in the art.

Among additional polynucleotides, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo.

In some cases, the polynucleotide containing nucleic acid sequences encoding the CD19-binding receptor, e.g., chimeric antigen receptor (CAR), contains a signal sequence that encodes a signal peptide. In some aspects, the signal sequence may encode a signal peptide derived from a native polypeptide. In other aspects, the signal sequence may encode a heterologous or non-native signal peptide. In some aspects, a non-limiting example of a signal peptide comprises a CD8 alpha (CD8a) signal peptide set forth in SEQ ID NO:4.

In some embodiments the vector or construct can contain promoter and/or enhancer or regulatory elements to regulate expression of the encoded recombinant receptor. In some examples the promoter and/or enhancer or regulatory elements can be condition-dependent promoters, enhancers, and/or regulatory elements. In some examples these elements drive expression of the transgene.

In some embodiments, the vector or construct can contain a single promoter that drives the expression of one or more nucleic acid molecules. In some embodiments, such nucleic acid molecules, e.g., transcripts, can be multicistronic (bicistronic or tricistronic). For example, in some embodiments, transcription units can be engineered as a bicistronic unit containing an IRES (internal ribosome entry site), which allows co-expression of gene products (e.g. encoding a chimeric receptor and membrane-bound interleukin-15) by a message from a single promoter. Alternatively, in some cases, a single promoter may direct expression of an RNA that contains, in a single open reading frame (ORF), two or three genes (e.g. encoding a chimeric receptor and membrane-bound interleukin-15) separated from one another by sequences encoding a self-cleavage peptide (e.g., 2A cleavage sequences) or a protease recognition site. The ORF thus encodes a single polypeptide, which, either during (in the case of T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream. Many 2A elements are known. Examples of 2A peptides that can be used in the methods and polynucleotides disclosed herein, without limitation, 2A peptides from the foot-and-mouth disease virus (F2A), equine rhinitis A virus (E2A), Thosea asigna virus (T2A, e.g. SEQ ID NO:20, encoded by SEQ ID NO: 19), and porcine teschovirus-1 (P2A). In some embodiments, the one or more different or separate promoters drive the expression of a nucleic acid molecule encoding a binding molecule, e.g., recombinant receptor and a nucleic acid encoding membrane-bound interleukin-15.

ii. Interleukin-15

In several embodiments, any of the immune cells as provided herein are engineered to express interleukin 15 (mbIL15). In some aspects, the IL15 is a membrane-bound form of IL15. Thus, in several embodiments, any of the immune cells as provided herein are engineered to express a membrane-bound interleukin 15 (mbIL15). In such embodiments, mbIL15 expression on the immune cell (e.g., NK cell) enhances the cytotoxic effects of the engineered cell by enhancing the proliferation and/or longevity of the cells. In some embodiments, the IL15 is expressed from a separate cassette on the construct comprising any one of the CARs disclosed herein. In some embodiments, the IL15 is expressed from the same cassette as any one of the CARs disclosed herein.

In some embodiments, the chimeric receptor and IL15 are separated by a nucleic acid sequence encoding a cleavage site, for example, a proteolytic cleavage site or a T2A. P2A, E2A, or F2A self-cleaving peptide cleavage site. In some embodiments, the chimeric receptor and IL15 are separated by a T2A peptide (e.g., SEQ ID NO:20, encoded by SEQ ID NO:19). In some embodiments, the IL15 is a membrane-bound IL15 (mbIL15). In some embodiments, the mbIL15 comprises a native IL15 sequence, such as a human native IL15 sequence (e.g., SEQ ID NO: 22, encoded by SEQ ID NO:21). In some embodiments, the mbIL15 comprises a native IL15 sequence, such as a human native IL15 sequence (e.g., SEQ ID NO:22, encoded by SEQ ID NO:21), and at least one transmembrane domain (e.g., CD8a). In several embodiments, IL15 is encoded by the nucleic acid sequence of SEQ ID NO: 21. In several embodiments, IL15 can be truncated or modified, such that it is encoded by a nucleic acid sequence that has at least 70%, at least 75%, at least 80%, at least 85%, at least 90%, at least 95% sequence identity to the nucleic acid sequence of SEQ ID NO: 21. In several embodiments, the IL15 comprises the amino acid sequence of SEQ ID NO: 22. In several embodiments, the IL15 is truncated or modified, such that it has at least 70%, at least 75%, at least 80%, at least 85%, at least 90%, at least 95% sequence identity to the amino acid sequence of SEQ ID NO: 22.

Thus, in some embodiments, any of the CARs as described herein are encoded by the same nucleic acid sequence as a mbIL15. In some embodiments, a nucleic acid sequence encoding the CAR and a nucleic acid sequence encoding the mbIL15 are separated by a T2A-encoding sequence (e.g., SEQ ID NO:19). In some embodiments, any of the engineered cells as described herein express a CD19-targeting recombinant receptor (e.g., CAR) and a mbIL15.

In some embodiments, the mbIL15 is membrane-bound by virtue of the fusion of IL15 to a transmembrane domain. Thus, in some embodiments, mbIL15 comprises a transmembrane domain. In some embodiments, the transmembrane domain comprises a CD8a transmembrane domain. In some embodiments, the transmembrane domain comprises a hinge and/or a transmembrane region. In some embodiments, the transmembrane domain comprises a hinge and a transmembrane region. In some embodiments, the hinge is a CD8a hinge sequence (e.g., SEQ ID NO:6). In some embodiments, the transmembrane region is a CD8a transmembrane region (e.g., SEQ ID NO:8). In some embodiments, the mbIL15 comprises a native IL15 sequence, such as a human native IL15 sequence, and at least one transmembrane domain (e.g., CD8a transmembrane domain). In some embodiments, the CD8a transmembrane domain comprises the sequence of SEQ ID NO:10. In several embodiments, the mbIL15 is truncated or modified such that it comprises an amino acid sequence that has at least 70%, at least 75%, at least 80%, at least 85%, at least 90%, at least 95% sequency identity to the amino acid sequence of SEQ ID NO:23. In several embodiments, the mbIL15 comprises the amino acid sequence of SEQ ID NO:23. Membrane-bound IL15 sequences are described in PCT publications WO 2018/183385 and WO 2020/056045, each of which is hereby expressly incorporated by reference in its entirety.

iii. Cell Types

Some embodiments of the methods and compositions provided herein relate to a cell such as an immune cell. In some embodiments, an immune cell is engineered to express a chimeric receptor that binds to an antigen (e.g., CD19).

Genetic engineering has enabled approaches to be developed that harness certain aspects of the immune system to fight disease. In some cases, a healthy donor's immune cells are modified to specifically eradicate cells of, or associated with, a disease (e.g., B cells associated with SLE). Various types of immune cells can be used, such as T cells, Natural Killer (NK cells), or combinations thereof, as described in more detail below.

To facilitate immunotherapies for treatment of autoimmune diseases, there are provided for herein polynucleotides, polypeptides, and vectors that encode chimeric antigen receptors (CAR) that comprise a target binding moiety (e.g., an antigen expressed by a B cell) and a cytotoxic signaling complex. For example, some embodiments include a polynucleotide, polypeptide, or vector that encodes, for example a chimeric antigen receptor directed against CD19, to facilitate targeting of an immune cell to e.g., B cells involved in autoimmune disease pathogenesis. Methods of treating autoimmune diseases (e.g., SLE) and other uses of such cells for immunotherapy are also provided for herein. Also provided are engineered immune cells (e.g., NK cells) expressing such chimeric receptors.

In several embodiments, cells of the immune system are engineered to have enhanced cytotoxic effects against target cells, such as tumor cells. For example, a cell of the immune system may be engineered to include a CD19-directed CAR as described herein. In several embodiments, white blood cells or leukocytes, are used, since their native function is to defend the body against growth of abnormal cells and infectious disease. There are a variety of types of white bloods cells that serve specific roles in the human immune system and are therefore a preferred starting point for the engineering of cells disclosed herein. White blood cells include granulocytes and agranulocytes (presence or absence of granules in the cytoplasm, respectively). Granulocytes include basophils, eosinophils, neutrophils, and mast cells. Agranulocytes include lymphocytes and monocytes. Cells such as those that follow or are otherwise described herein may be engineered to include a CD19-directed CAR, or a nucleic acid encoding the CAR. In several embodiments, the immune cells are engineered to co-express a membrane-bound interleukin 15 (mbIL15) co-stimulatory domain. In some embodiments, the immune cells engineered to express a CAR are engineered to bicistronically express a mbIL15 domain.

a. Monocytes

In some embodiments, the immune cells comprise monocytes. Monocytes are a subtype of leukocyte. Monocytes can differentiate into macrophages and myeloid lineage dendritic cells. Monocytes are associated with the adaptive immune system and serve the main functions of phagocytosis, antigen presentation, and cytokine production. Phagocytosis is the process of uptake of cellular material, or entire cells, followed by digestion and destruction of the engulfed cellular material.

In some embodiments, a monocyte is positive for cell surface expression of a marker selected from among the group consisting of CCR2, CCR5, CD11c, CD14, CD16, CD62L, CD68+, CX3CR1, HLA-DR, or any combination thereof. In some embodiments, a monocyte is positive for cell surface expression of CD14. In some embodiments, a monocyte is positive for cell surface expression of CCR2. In some embodiments, a monocyte is positive for cell surface expression of CCR5. In some embodiments, a monocyte is positive for cell surface expression of CD62L.

In several embodiments, monocytes are used in connection with one or more additional engineered cells as disclosed herein. Some embodiments of the methods and compositions described herein relate to a monocyte that expresses a CAR that binds to CD19, or a nucleic acid encoding the CAR.

In some embodiments, the monocytes are engineered to express a membrane-bound interleukin 15 (mbIL15) domain. In some embodiments, the monocytes engineered to express a CAR are engineered to also express (e.g., bicistronically express) a membrane-bound interleukin 15 (mbIL15) domain. Thus, in some embodiments, the monocytes are engineered to bicistronically express the CAR and mbIL15.

In some embodiments, the monocytes are allogeneic cells. In some embodiments, the monocytes are obtained from a donor who does not have an autoimmune disease.

b. Lymphocytes

In some embodiments, the immune cells comprise lymphocytes. Lymphocytes, the other primary sub-type of leukocyte include T cells (cell-mediated, cytotoxic adaptive immunity), natural killer cells (cell-mediated, cytotoxic innate immunity), and B cells (humoral, antibody-driven adaptive immunity). While B cells are engineered according to several embodiments, disclosed herein, several embodiments also relate to engineered T cells or engineered NK cells (mixtures of T cells and NK cells are used in some embodiments, either from the same donor, or different donors). Thus, in some embodiments, the immune cells comprise T cells. In some embodiments, the immune cells comprise NK cells. In some embodiments, the immune cells comprise T cells and NK cells. In some embodiments, the immune cells comprise B cells.

In several embodiments, lymphocytes are used in connection with one or more additional engineered cells as disclosed herein. Some embodiments of the methods and compositions described herein relate to lymphocytes that express a CAR that binds to CD19, or a nucleic acid encoding the CAR.

In some embodiments, the lymphocytes are engineered to express a membrane-bound interleukin 15 (mbIL15) domain. In some embodiments, the lymphocytes engineered to express a CAR are engineered to also express (e.g., bicistronically express) a membrane-bound interleukin 15 (mbIL15) domain. Thus, in some embodiments, lymphocytes are engineered to bicistronically express the CAR and mbIL15.

In some embodiments, the lymphocytes are allogeneic cells. In some embodiments, the monocytes are obtained from a donor who does not have an autoimmune disease.

c. T Cells

In some embodiments, the immune cells comprise T cells. T cells are distinguishable from other lymphocytes sub-types (e.g., B cells or NK cells) based on the presence of a T-cell receptor on the cell surface.

T cells can be divided into various different subtypes, including effector T cells, helper T cells, cytotoxic T cells, memory T cells, regulatory T cells, natural killer T cell, mucosal associated invariant T cells and gamma delta T cells. In some embodiments, a specific subtype of T cell is engineered. In some embodiments, a T cell is positive for cell surface expression of a marker selected from among the group consisting of CD3, CD4, and/or CD8. In some embodiments, a T cell is positive for cell surface expression of CD3. In some embodiments, a T cell is positive or cell surface expression of CD4. In some embodiments, a T cell is positive or cell surface expression of CD8.

In some embodiments, CD3+ T cells are engineered. In some embodiments, CD4+ T cells are engineered. In some embodiments, CD8+ T cells are engineered. In some embodiments, regulatory T cells are engineered. In some embodiments, gamma delta T cells are engineered. In some embodiments, a mixed pool of T cell subtypes is engineered. For example, in some embodiments, CD4+ and CD8+ T cells are engineered. In some embodiments, there is no specific selection of a type of T cells to be engineered to express the cytotoxic receptor complexes disclosed herein. In several embodiments, specific techniques, such as use of cytokine stimulation are used to enhance expansion/collection of T cells with a specific marker profile. For example, in several embodiments, activation of certain human T cells, e.g. CD4+ T cells, CD8+ T cells is achieved through use of CD3 and/or CD28 as stimulatory molecules.

In several embodiments, there is provided a method of treating or preventing an autoimmune disease, comprising administering T cells expressing a cytotoxic receptor complex as described herein. In several embodiments, the engineered T cells are autologous cells, while in some embodiments, the T cells are allogeneic cells. In some embodiments, the T cells are allogeneic cells. In some embodiments, the T cells are obtained from a donor who does not have an autoimmune disease.

Several embodiments of the methods and compositions disclosed herein relate to T cells engineered to express a CAR that binds to CD19. In some embodiments, the T cells are engineered to express a membrane-bound interleukin 15 (mbIL15) domain. In some embodiments, the T cells engineered to express a CAR are engineered to also express (e.g., bicistronically express) a membrane-bound interleukin 15 (mbIL15) domain. Thus, in some embodiments, the T cells are engineered to bicistronically express the CAR and mbIL15.

In some embodiments, the immune cells comprise T cells and NK cells (either from the same donor or from different donors).

d. NK Cells

In some embodiments, the immune cells comprise natural killer (NK) cells. In several embodiments, there is provided a method of treating or preventing an autoimmune disease, comprising administering NK cells expressing a CD19-targeting CAR as described herein. In several embodiments, the engineered NK cells are autologous cells, while in some embodiments, the NK cells are allogeneic cells. In some embodiments, the NK cells are allogeneic. In some embodiments, the NK cells are derived from a donor who does not have an autoimmune disease. In some embodiments, the NK cells are derived from a donor who does not have SLE.

In several embodiments, NK cells are preferred because the natural cytotoxic potential of NK cells is relatively high. In several embodiments, it is unexpectedly beneficial that the engineered cells disclosed herein can further upregulate the cytotoxic activity of NK cells, leading to an even more effective activity against target cells (e.g., tumor or other diseased cells).

In some embodiments, a NK cell is positive for cell surface expression of a marker selected from among the group consisting of CCR7, CD16, CD56, CD57, CD11, CX3CR1, a Killer Ig-like receptor (KIR), NKp30, NKp44, NKp46, or any combination thereof. In some embodiments, a NK cell is positive for cell surface expression of CD16. In some embodiments, a NK cell is positive for cell surface expression of CD56. In some embodiments, a NK cell is positive for cell surface expression of a Killer Ig-like receptor.

Some embodiments of the methods and compositions described herein relate to NK cells engineered to express a CAR that binds to CD19. In some embodiments, the NK cells are engineered to a membrane-bound interleukin 15 (mbIL15) domain. In some embodiments, the NK cells engineered to express a CAR are engineered to also express (e.g., bicistronically express) a membrane-bound interleukin 15 (mbIL15) domain. Thus, in some embodiments, the NK cells are engineered to bicistronically express the CAR and mbIL15.

In some embodiments, the NK cells are derived from cell line NK-92. NK-92 cells are derived from NK cells, but lack major inhibitory receptors displayed by normal NK cells, while retaining the majority of activating receptors. Some embodiments of NK-92 cells described herein related to NK-92 cell engineered to silence certain additional inhibitory receptors, for example, SMAD3, allowing for upregulation of interferon-γ (IFNγ), granzyme B, and/or perforin production. Additional information relating to the NK-92 cell line is disclosed in WO 1998/49268 and U.S. Patent Application Publication No. 2002-0068044 and incorporated in their entireties herein by reference.

In some embodiments, the NK cells are used in combination with T cells. Thus, in some embodiments, the immune cells comprise T cells and NK cells (either from the same donor or from different donors). For example, in one embodiment, primary NK cells are used in combination with primary T cells.

In any of the provided embodiments, the NK cells engineered to express a CD19 CAR are further engineered to express a CAR that binds to an antigen other than CD19. In some embodiments, the antigen is associated with an autoimmune disease. For example, in some embodiments, the genetically engineered NK cells also express a CAR that binds to an antigen selected from the group consisting of BAFF-R, BCMA, CD20, CD22, CD27, CD28, CD33, CD38, CD45, CD47, CD54, CD56, CD81, CD117, CD138, CD200, FcRH5, GPRC5D, and SLAMF7. Thus, in some aspects, the NK cells are engineered to express an anti-CD19 CAR as provided herein and a CAR that binds to any one of BAFF-R, BCMA, CD20, CD22, CD27, CD28, CD33, CD38, CD45, CD47, CD54, CD56, CD81, CD117, CD138, CD200, FcRH5, GPRC5D, and SLAMF7. For example, in some embodiments, the NK cells are engineered to express a CAR that binds to CD19 and a CAR that binds to BCMA. Anti-BCMA CARs are known in the art and include any of those described in PCT Application No. PCT/US2022/073567. In some embodiments, the NK cells are engineered to express a CAR that binds to CD19 and a CAR that binds to BAFF-R. In some embodiments, the NK cells are engineered to express a CAR that binds to CD19 and a CAR that binds to CD20. In some embodiments, the NK cells are engineered to express a CAR that binds to CD19 and a CAR that binds to CD22. In some embodiments, the NK cells are engineered to express a CAR that binds to CD19 and a CAR that binds to CD27. In some embodiments, the NK cells are engineered to express a CAR that binds to CD19 and a CAR that binds to CD28. In some embodiments, the NK cells are engineered to express a CAR that binds to CD19 and a CAR that binds to CD33. In some embodiments, the NK cells are engineered to express a CAR that binds to CD19 and a CAR that binds to CD38. In some embodiments, the NK cells are engineered to express a CAR that binds to CD19 and a CAR that binds to CD45. In some embodiments, the NK cells are engineered to express a CAR that binds to CD19 and a CAR that binds to CD47. In some embodiments, the NK cells are engineered to express a CAR that binds to CD19 and a CAR that binds to CD54. In some embodiments, the NK cells are engineered to express a CAR that binds to CD19 and a CAR that binds to CD56. In some embodiments, the NK cells are engineered to express a CAR that binds to CD19 and a CAR that binds to CD81. In some embodiments, the NK cells are engineered to express a CAR that binds to CD19 and a CAR that binds to CD117. In some embodiments, the NK cells are engineered to express a CAR that binds to CD19 and a CAR that binds to CD138. In some embodiments, the NK cells are engineered to express a CAR that binds to CD19 and a CAR that binds to CD200. In some embodiments, the NK cells are engineered to express a CAR that binds to CD19 and a CAR that binds to FcRH5. In some embodiments, the NK cells are engineered to express a CAR that binds to CD19 and a CAR that binds to GPRC5D. In some embodiments, the NK cells are engineered to express a CAR that binds to CD19 and a CAR that binds to SLAMF7.

c. Hematopoietic Stem Cells

In some embodiments, the immune cells comprise hematopoietic stem cells (HSCs). In some embodiments, HSCs are used in the methods disclosed herein. In several embodiments, the cells are engineered to express a CAR that binds to CD19.

In some embodiments, a HSC is positive for cell surface expression of a marker selected from among the group consisting of CD34, CD59, and CD90. In some embodiments, a HSC is positive for cell surface expression of CD34. In some embodiments, a HSC is positive for cell surface expression of CD59. In some embodiments, a HSC is positive for cell surface expression of CD90.

In several embodiments allogeneic HSCs are used, while in some embodiments, autologous HSCs are used. In several embodiments, HSCs are used in combination with one or more additional engineered cell type disclosed herein. Some embodiments of the methods and compositions described herein relate to a stem cell, such as a HSC engineered to express a CAR that binds to CD19, or a nucleic acid encoding the CAR.

In some embodiments, the HSCs are engineered to express a membrane-bound interleukin 15 (mbIL15) domain. In some embodiments, the HSCs engineered to express a CAR are engineered to also express (e.g., bicistronically express) a membrane-bound interleukin 15 (mbIL15) domain. Thus, in some embodiments, HSCs are engineered to bicistronically express the CAR and mbIL15.

In some embodiments, the HSCs are allogeneic cells. In some embodiments, the HSCs are obtained from a donor who does not have an autoimmune disease.

f. Induced Pluripotent Stem Cells

In some embodiments, immune cells are derived (differentiated) from pluripotent stem cells (PSCs). In some embodiments, immune cells (e.g., NK cells) derived from induced pluripotent stem cells (iPSCs) are used in the method of immunotherapy disclosed herein. For example, in some embodiments, NK cells are derived from iPSCs. In some embodiments, induced pluripotent stem cells (iPSCs) are used in a method disclosed herein. iPSCs are used, in several embodiments, to leverage their ability to differentiate and derive into non-pluripotent cells, including, but not limited to, CD34 cells, hemogenic endothelium cells, HSCs (hematopoietic stem and progenitor cells), hematopoietic multipotent progenitor cells, T cell progenitors, NK cell progenitors, T cells, NKT cells, NK cells, and B cells comprising one or several genetic modifications at selected sites through differentiating iPSCs or less differentiated cells comprising the same genetic modifications at the same selected sites. In several embodiments, the iPSCs are used to generate iPSC-derived NK cells.

Several embodiments of the methods and compositions disclosed herein relate to induced pluripotent stem cells engineered to express a CAR that binds to CD19. In some embodiments, the iPSCs engineered to express a CAR are engineered to also express (e.g., bicistronically express) a membrane-bound interleukin 15 (mbIL15).

In several embodiments, the engineered iPSCs are differentiated into NK, T. or other immune cells, such as for use in a composition or method provided herein. In several embodiments, the engineered iPSCs are differentiated into NK cells.

### C. Preparation of Cells for Genetic Engineering

In some embodiments, preparation of the engineered cells includes one or more culture and/or preparation steps. The cells for introduction of the recombinant receptor (e.g., CAR) may be isolated from a sample, such as a biological sample, e.g., one obtained from or derived from a subject. In some embodiments, the sample is an apheresis (e.g., leukapheresis) sample.

In some embodiments, the subject from which the cells are isolated is one not having the autoimmune disease or in need of a cell therapy or not to which a cell therapy will be administered. In some embodiments, the cells are isolated from a subject that is different than the subject in need of a cell therapy or to which a cell therapy will be administered. Thus, in some embodiments, the cells are allogeneic to the subject to whom they are administered.

In some embodiments, the subject from which the cells are isolated is one having the autoimmune disease or in need of a cell therapy or to which a cell therapy will be administered. In some embodiments, the cells are isolated from the subject to which a cell therapy will be administered. Thus, in some embodiments, the cells are autologous to the subject to whom they are administered.

The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis (e.g., a leukapheresis) product. In some embodiments, the cells are isolated from an apheresis (e.g., leukapheresis) sample. Non-limiting samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. In some embodiments, the cells are derived from PBMCs. Samples include, in the context of cell therapy, e.g., adoptive cell therapy, samples from autologous and allogeneic sources.

The cells in some embodiments are primary cells, e.g., primary human cells. In some embodiments, the cells are immune cells, e.g. primary NK cells.

In some embodiments, isolation of the cells includes one or more preparation and/or non affinity-based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis (e.g., leukapheresis). The samples, in some aspects, contain lymphocytes, including NK cells, T cells, monocytes, granulocytes, B cells, other nucleated white blood cells, red blood cells, and/or platelets, and in some aspects contain cells other than red blood cells and platelets.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, NK cells or specific subpopulations thereof, such as cells positive or expressing high levels of one or more surface markers, e.g., CD56+, CCR7+, CD16+, CD57+, CD11+, CX3CR1+, a Killer Ig-like receptor (KIR)+, NKp30+, NKp44+, or NKp46+NK cells, are isolated by positive or negative selection techniques. In some aspects, NK cells are isolated by positive selection for CD56. For example, CD56+NK cells can be positively selected using anti-CD56 conjugated magnetic beads.

In some embodiments, the cells (e.g., NK cells) are expanded in culture prior to, during, and/or following genetic engineering. In some embodiments, the cells are expanded in culture prior to genetic engineering. In some embodiments, the cells are expanded in culture following genetic engineering. In some embodiments, the cells are expanded in culture prior to and following genetic engineering. Methods for expanding cells are known in the art and include any of those described in U.S. Pat. Nos. 7,435,596 and 8,026,097; and Patent Application Nos. PCT/SG2018/050138; PCT/US2020/044033; PCT/US2021/071330; and PCT/US2022/074164.

In some embodiments, expanding the cells in culture comprises co-culturing the cells with feeder (e.g., stimulatory) cells. Thus, in some embodiments, the cells are expanded in culture prior to genetic engineering by co-culturing the cells with feeder cells. In some embodiments, the feeder cells express IL15 (e.g., membrane-bound IL15) and 4-1BBL. In some embodiments, the feeder cells express membrane-bound interleukin 15 (mbIL15) and 4-1BBL. In some embodiments, the feeder cells do not express MHCI molecules. In some embodiments, the feeder cells do not express MHCII molecules. In some embodiments, the feeder cells are immune cells. In some embodiments, the feeder cells are K562 cells. Engineered feeder cells are disclosed in, for example, International Patent Application PCT/SG2018/050138. In some embodiments, following genetic engineering, the cells are allowed to further expand in culture.

In some embodiments, expanding the cells in culture comprises culturing the cells in the presence of IL2, IL12, and/or IL18. In some embodiments, the cells are cultured in the presence of IL2. In some embodiments, the cells are cultured in the presence of IL12 and IL18. In some embodiments, the cells are cultured in the presence of IL2, IL12, and IL18.

In some embodiments, the preparation methods include steps for freezing, e.g., cryopreserving, the cells, either before or after isolation, engineering, and/or expansion. In some aspects, the cells are cryopreserved after engineering. In some aspects, such as when the cells are further expanded in culture after genetic engineering, the cells are cryopreserved after the further expansion. In some embodiments, the cells are suspended in a freezing solution. In some embodiments, a composition provided herein is cryopreserved (e.g., prior to infusion into a subject). Any of a variety of known freezing solutions and parameters in some aspects may be used.

### D. Genetic Editing of Cells

Provided are methods and uses of genetically engineered immune cells, including genetically engineered immune cells that are genetically edited, and/or compositions thereof. In some aspects, the immune cells are genetically edited to increase or decrease expression of a target protein. In some aspects, the immune cells are genetically edited to increase expression of a target protein. In some aspects, the immune cells are genetically edited to decrease expression of a target protein. In some aspects, the methods comprise genetically editing the immune cells, such as to increase or decrease expression of a target protein. In some aspects, the methods comprise genetically editing the immune cells to increase expression of a target protein. In some aspects, the methods comprise genetically editing the immune cells to decrease expression of a target protein. Expression of a target protein can be reduced by disrupting a gene (a target gene) encoding the target protein or a portion thereof.

It is contemplated that the immune cells can be genetically edited at any point prior to, during, and/or after the genetic engineering. In some embodiments, the immune cells are genetically edited prior to the genetic engineering. In some embodiments, the immune cells are genetically edited contemporaneously with the genetic engineering. In some embodiments, the immune cells are genetically edited after the genetic engineering.

As discussed below, in several embodiments, genetic editing is employed to reduce or eliminate expression of a target protein, for example by disrupting a gene encoding the protein. In several embodiments, genetic editing can reduce transcription of a target gene by about 30%, about 40%, about 50%, about 60%, about 70%, about 75%, about 80%, about 85%, about 90%, about 95%, about 97%, about 98%, about 99%, or more (including any amount between those listed). In several embodiments, genetic editing reduces transcription of a target gene by at least about 30%. In several embodiments, genetic editing reduces transcription of a target gene by at least about 40%. In several embodiments, genetic editing reduces transcription of a target gene by at least about 50%. In several embodiments, genetic editing reduces transcription of a target gene by at least about 60%. In several embodiments, genetic editing reduces transcription of a target gene by at least about 70%. In several embodiments, genetic editing reduces transcription of a target gene by at least about 80%. In several embodiments, genetic editing reduces transcription of a target gene by at least about 90%. In several embodiments, genetic editing reduces transcription of a target gene by at least about 95%. In several embodiments, genetic editing reduces transcription of a target gene by at least about 99%. In several embodiments, the gene is completely knocked out, such that transcription of the target gene is eliminated (undetectable).

In several embodiments, genetic editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, genetic editing reduces expression of a target protein by at least about 30%. In several embodiments, genetic editing reduces expression of a target protein by at least about 40%. In several embodiments, genetic editing reduces expression of a target protein by at least about 50%. In several embodiments, genetic editing reduces expression of a target protein by at least about 60%. In several embodiments, genetic editing reduces expression of a target protein by at least about 70%. In several embodiments, genetic editing reduces expression of a target protein by at least about 80%. In several embodiments, genetic editing reduces expression of a target protein by at least about 90%. In several embodiments, genetic editing reduces expression of a target protein by at least about 95%. In several embodiments, genetic editing reduces expression of a target protein by at least about 99%. In several embodiments, the gene is completely knocked out, such that expression of the target protein is eliminated (undetectable).

In several embodiments, genetic editing is used to “knock in” or otherwise increase transcription of a target gene. In several embodiments, transcription of a target gene is increased by about 30%, about 40%, about 50%, about 60%, about 70%, about 75%, about 80%, about 85%, about 90%, about 95%, about 97%, about 98%, about 99%, or more (including any amount between those listed). In several embodiments, transcription of a target gene is increased by at least about 30%. In several embodiments, transcription of a target gene is increased by at least about 40%. In several embodiments, transcription of a target gene is increased by at least about 50%. In several embodiments, transcription of a target gene is increased by at least about 60%. In several embodiments, transcription of a target gene is increased by at least about 70%. In several embodiments, transcription of a target gene is increased by at least about 80%. In several embodiments, transcription of a target gene is increased by at least about 90%. In several embodiments, transcription of a target gene is increased by at least about 100%.

In several embodiments, genetic editing is used to “knock in” or otherwise enhance expression of a target protein. In several embodiments, expression of a target protein can be enhanced by about 30%, about 40%, about 50%, about 60%, about 70%, about 75%, about 80%, about 85%, about 90%, about 95%, about 97%, about 98%, about 99%, or more (including any amount between those listed). In several embodiments, expression of a target protein is increased by at least about 30%. In several embodiments, expression of a target protein is increased by at least about 40%. In several embodiments, expression of a target protein is increased by at least about 50%. In several embodiments, expression of a target protein is increased by at least about 60%. In several embodiments, expression of a target protein is increased by at least about 70%. In several embodiments, expression of a target protein is increased by at least about 80%. In several embodiments, expression of a target protein is increased by at least about 90%. In several embodiments, expression of a target protein is increased by at least about 100%.

As discussed in more detail below, a variety of approaches can be employed in a given embodiment to improve or alter one or more characteristics of immune cells. Genetic editing can be used to reduce, eliminate (e.g., knockout), or increase expression of a target gene. For example, the transcription of the target gene and/or the translation of a protein encoded by the target gene (e.g., a target protein) can be reduced, eliminated (e.g., knocked out), or increased. The target gene can be implicated in the immune functionality of the cell or be a part of a signaling pathway for which an increase or decrease in function is desired. Further detailed below are various gene targets. Disruption of certain genes in immune cells (e.g., NK cells) can increase activity and/or persistence of those immune cells.

i. Methods of Genetic Editing

In several embodiments, genetic editing (whether knock out or knock in) of a target gene is accomplished through targeted introduction of DNA breakage, and a subsequent DNA repair mechanism. In several embodiments, double strand breaks of DNA are repaired by non-homologous end joining (NHEJ), wherein enzymes are used to directly join the DNA ends to one another to repair the break. NHEJ is an error-prone process. In general, in the absence of a repair template, the NHEJ process re-ligates the ends of the cleaved DNA strands, which frequently results in nucleotide deletions and insertions at the cleavage site. In several embodiments, however, double strand breaks are repaired by homology directed repair (HDR), which is advantageously more accurate, thereby allowing sequence specific breaks and repair. HDR uses a homologous sequence as a template for regeneration of missing DNA sequences at the break point, such as a vector with the desired genetic elements (e.g., an insertion element to disrupt the coding sequence of a TCR subunit) within a sequence that is homologous to the flanking sequences of a double strand break. This will result in the desired change (e.g., insertion) being inserted at the site of the DSB. The HDR pathway can occur by way of the canonical HDR pathway or the alternative HDR pathway. Unless otherwise indicated, the term “HDR” or “homology-directed repair” as used herein encompasses both canonical HDR and alternative HDR.

Canonical HDR or “canonical homology-directed repair” or “cHDR,” are used interchangeably, and refers to the process of repairing DNA damage using a homologous nucleic acid (e.g., an endogenous homologous sequence, such as a sister chromatid; or an exogenous nucleic acid, such as a donor template). Canonical HDR typically acts when there has been a significant resection at the DSB, forming at least one single-stranded portion of DNA. In a normal cell, canonical HDR typically involves a series of steps such as recognition of the break, stabilization of the break, resection, stabilization of single-stranded DNA, formation of a DNA crossover intermediate, resolution of the crossover intermediate, and ligation. The canonical HDR process requires RAD51 and BRCA2, and the homologous nucleic acid, e.g., repair template, is typically double-stranded. In canonical HDR, a double-stranded polynucleotide, e.g., a double-stranded repair template, is introduced, which comprises a sequence that is homologous to the targeting sequence, and which will either be directly integrated into the targeting sequence or will be used as a template to insert the sequence, or a portion the sequence, of the repair template into the target gene. After resection at the break, repair can progress by different pathways, e.g., by the double Holliday junction model (also referred to as the double strand break repair, or DSBR, pathway), or by the synthesis-dependent strand annealing (SDSA) pathway.

In the double Holliday junction model, strand invasion occurs by the two single stranded overhangs of the targeting sequence to the homologous sequences in the double-stranded polynucleotide, e.g., double stranded donor template, which results in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in the insertion at the targeting sequence, or a portion of the targeting sequence that includes the gene variant. Crossover with the polynucleotide, e.g., repair template, may occur upon resolution of the junctions.

In the SDSA pathway, only one single stranded overhang invades the polynucleotide, e.g., donor template, and new DNA is synthesized from the end of the invading strand to fill the gap resulting from resection. The newly synthesized DNA then anneals to the remaining single stranded overhang, new DNA is synthesized to fill in the gap, and the strands are ligated to produce the modified DNA duplex.

Alternative HDR, or “alternative homology-directed repair,” or “alternative HDR,” are used interchangeably, and refers, in some embodiments, to the process of repairing DNA damage using a homologous nucleic acid (e.g., an endogenous homologous sequence, such as a sister chromatid; or an exogenous nucleic acid, such as a repair template). Alternative HDR is distinct from canonical HDR in that the process utilizes different pathways from canonical HDR, and can be inhibited by the canonical HDR mediators, RAD51 and BRCA2. Moreover, alternative HDR is also distinguished by the involvement of a single-stranded or nicked homologous nucleic acid template, e.g., repair template, whereas canonical HDR generally involves a double-stranded homologous template. In the alternative HDR pathway, a single strand template polynucleotide, e.g., repair template, is introduced. A nick, single strand break, or DSB at the cleavage site, for altering a desired target site, e.g., a gene variant in a target gene, is mediated by a nuclease molecule, and resection at the break occurs to reveal single stranded overhangs. Incorporation of the sequence of the template polynucleotide, e.g., repair template, to alter the target site of the DNA typically occurs by the SDSA pathway, as described herein. In some embodiments, HDR is carried out by introducing, into a cell, one or more agent(s) capable of inducing a DSB, and a repair template, e.g., a single-stranded oligonucleotide. The introducing can be carried out by any suitable delivery. The conditions under which HDR is allowed to occur can be any conditions suitable for carrying out HDR in a cell.

In several embodiments, gene editing is accomplished by one or more of a variety of engineered nucleases. In several embodiments, restriction enzymes are used, particularly when double strand breaks are desired at multiple regions. In several embodiments, a bioengineered nuclease is used. Depending on the embodiment, one or more of a Zinc Finger Nuclease (ZFN), transcription-activator like effector nuclease (TALEN), meganuclease and/or clustered regularly interspaced short palindromic repeats (CRISPR/Cas9) system are used to specifically edit the genes encoding one or more of the TCR subunits.

Meganucleases are characterized by their capacity to recognize and cut large DNA sequences (from 14 to 40 base pairs). In several embodiments, a meganuclease from the LAGLIDADG family is used, and is subjected to mutagenesis and screening to generate a meganuclease variant that recognizes a unique sequence(s), such as a specific site in a target gene, or any other target gene disclosed herein. In several embodiments, two or more meganucleases, or functions fragments thereof, are fused to create a hybrid enzyme that recognizes a desired target sequence within the target gene.

In contrast to meganucleases, ZFNs and TALEN function based on a non-specific DNA cutting catalytic domain which is linked to specific DNA sequence recognizing peptides such as zinc fingers or transcription activator-like effectors (TALEs). Advantageously, the ZFNs and TALENs thus allow sequence-independent cleavage of DNA, with a high degree of sequence-specificity in target recognition. Zinc finger motifs naturally function in transcription factors to recognize specific DNA sequences for transcription. The C-terminal part of each finger is responsible for the specific recognition of the DNA sequence. While the sequences recognized by ZFNs are relatively short, (e.g., ˜3 base pairs), in several embodiments, combinations of 2, 3, 4, 5, 6, 7, 8, 9, 10 or more zinc fingers whose recognition sites have been characterized are used, thereby allowing targeting of specific sequences. The combined ZFNs are then fused with the catalytic domain(s) of an endonuclease, such as FokI (optionally a FokI heterodimer), to induce a targeted DNA break.

Transcription activator-like effector nucleases (TALENs) are specific DNA-binding proteins that feature an array of 33 or 34-amino acid repeats. Like ZFNs, TALENs are a fusion of a DNA cutting domain of a nuclease to TALE domains, which allow for sequence-independent introduction of double stranded DNA breaks with highly precise target site recognition. TALENs can create double strand breaks at the target site that can be repaired by error-prone non-homologous end-joining (NHEJ), resulting in gene disruptions through the introduction of small insertions or deletions. Advantageously, TALENs are used in several embodiments, at least in part due to their higher specificity in DNA binding, reduced off-target effects, and ease in construction of the DNA-binding domain.

CRISPRs (Clustered Regularly Interspaced Short Palindromic Repeats) are genetic elements that bacteria use as protection against viruses. The repeats are short sequences that originate from viral genomes and have been incorporated into the bacterial genome. Cas (CRISPR associated proteins) process these sequences and cut matching viral DNA sequences. By introducing plasmids containing Cas genes and specifically constructed CRISPRs into eukaryotic cells, the eukaryotic genome can be cut at any desired position. Additional information on CRISPR can be found in US Patent Publication No. 2014/0068797, which is incorporated by reference herein.

In several embodiments, CRISPR is used to disrupt a target gene. Depending on the embodiment and which target gene is to be edited, a Class 1 or Class 2 Cas is used. In several embodiments, a Class 1 Cas is used, and the Cas type is selected from the following types: I, IA, IB, IC, ID, IE, IF, IU, III, IIIA, IIIB, IIIC, IIID, IV IVA, IVB, and combinations thereof. In several embodiments, the Cas is selected from the group consisting of Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof. In several embodiments, the Cas is Cas3. In several embodiments, a Class 2 Cas is used, and the Cas type is selected from the following types: II, IIA, IIB, IIC, V, VI, and combinations thereof. In several embodiments, the Cas is selected from the group consisting of Cas9, Csn2, Cas4, Cas12a (previously known as Cpf1), C2c1, C2c3, Cas13a (previously known as C2c2), Cas13b, Cas13c, CasX, CasY and combinations thereof. In some embodiments, the Cas is Cas9. In some embodiments, class 2 CasX is used, wherein CasX can form a complex with a guide nucleic acid and wherein the complex can bind to a target DNA, and wherein the target DNA comprises a non-target strand and a target strand. In some embodiments, class 2 CasY is used, wherein CasY is capable of binding and modifying a target nucleic acid and/or a polypeptide associated with target nucleic acid.

ii. Target Genes

In some embodiments, the immune cells are genetically edited at a target gene. In several embodiments, editing of a target gene advantageously imparts to the edited cells enhanced expansion, cytotoxicity and/or persistence. For example, in some embodiments, immune cells are genetically edited to increase IL15 levels and/or signaling. Without wishing to be bound by theory, it is contemplated that genetically editing the cells to increase IL15 levels and/or signaling may obviate the need to provide a lymphodepleting therapy containing fludarabine to subjects prior to administration of genetically engineered cells. Specifically, it is contemplated that the increased IL15 bioavailability afforded by fludarabine is not necessary in immune cells genetically edited to increase IL15 levels and/or signaling. Thus, in some embodiments, the immune cells (e.g., NK cells) are genetically edited at a target gene to increase IL15 levels, such as by reducing or eliminating expression of the cytokine-inducible SH2-containing protein (Cis) (e.g., by disrupting the CISH gene encoding Cis). In some aspects, the immune cells (e.g., NK cells) are genetically edited at a target gene to increase IL15 signaling, such as by reducing or eliminating expression of the cytokine-inducible SH2-containing protein (Cis) (e.g., by disrupting the CISH gene encoding Cis).

By way of non-limiting example, IL15 is a positive regulator of NK cells, which as disclosed herein, can enhance one or more of NK cell homing, NK cell migration, NK cell expansion/proliferation, NK cell cytotoxicity, and/or NK cell persistence. In CD8+ T cells, CISH actively silences TCR signaling to maintain tumor tolerance, and CISH has been shown to be a downstream negative regulator of IL-15 receptor signaling (Palmer et al., J. Exp. Med. (2015) 212 (12): 2095-2113). In NK and T cells, CISH plays a role in checkpoint maturation and proliferation (Delconte et al., Nature Immunol (2016) 17:816-24). Thus, according to several embodiments, genetically editing CISH increases the persistence, proliferation, and/or cytotoxicity, or otherwise enhances the efficacy, of immune cells (e.g., NK cells) as disclosed herein.

In several embodiments, CISH genetic editing activates or inhibits a wide variety of pathways. The CIS protein is a negative regulator of IL15 signaling by way of, for example, inhibiting JAK-STAT signaling pathways. These pathways would typically lead to transcription of IL15-responsive genes (including CISH). In several embodiments, disruption of CISH disinhibits JAK-STAT (e.g., JAK1-STAT5) signaling and there is enhanced transcription of IL15-responsive genes. In several embodiments, disruption of CISH yields enhanced signaling through mammalian target of rapamycin (mTOR), with corresponding increases in expression of genes related to cell metabolism and respiration. In several embodiments, disruption of CISH yields IL15 induced increased expression of IL-2Rα (CD25), but not IL-15Rα or IL-2/15Rβ, enhanced NK cell membrane binding of IL15 and/or IL2, increased phosphorylation of STAT-3 and/or STAT-5, and elevated expression of the antiapoptotic proteins, such as Bel-2. In several embodiments, CISH disruption results in IL15-induced upregulation of selected genes related to mitochondrial functions (e.g., electron transport chain and cellular respiration) and cell cycle. Thus, in several embodiments, CISH disruption by genetic editing enhances the NK cell cytotoxicity and/or persistence, at least in part via metabolic reprogramming. In several embodiments, negative regulators of cellular metabolism, such as TXNIP, are downregulated in response to CISH disruption. In several embodiments, promotors for cell survival and proliferation including BIRC5 (Survivin), TOP2A, CKS2, and RACGAP1 are upregulated after CISH disruption, whereas antiproliferative or proapoptotic proteins such as TGFB1, ATM, and PTCH1 are downregulated. In several embodiments, CISH disruption alters the state (e.g., activates or inactivates) signaling via or through one or more of CXCL-10, IL2, TNF, IFNg, IL13, IL4, Jnk, PRF1, STAT5, PRKCQ, IL2 receptor Beta, SOCS2, MYD88, STAT3, STAT1, TBX21, LCK, JAK3, IL& receptor, ABL1, IL9, STAT5A, STAT5B, Tcf7, PRDM1, and/or EOMES.

In several embodiments, CISH editing endows an NK cell with enhanced ability to home to a target site. In several embodiments, CISH editing endows an NK cell with enhanced ability to migrate, e.g., within a tissue in response to, for example chemoattractants or away from repellants. In several embodiments, CISH editing endows an NK cell with enhanced ability to be activated, and thus exert, for example, anti-tumor effects. In several embodiments, CISH editing endows an NK cell with enhanced proliferative ability, which in several embodiments, allows for generation of robust NK cell numbers from a donor blood sample. In addition, in such embodiments, NK cells edited for CISH and engineered to express a CAR are more readily, robustly, and consistently expanded in culture. In several embodiments, CISH genetic editing endows an NK cell with enhanced cytotoxicity. In several embodiments, the editing of CISH synergistically enhances the cytotoxic effects of immune cells that express a CAR.

In several embodiments, CIS expression is knocked down or knocked out through genetic editing of the CISH gene, for example, by use of CRISPR-Cas editing. Thus, in some embodiments, the immune cells (e.g., NK cells) are genetically edited at the CISH gene. Small interfering RNA, antisense RNA. TALENs or zinc fingers are used in other embodiments. Information on CISH editing can be found, for example, in International Patent Application Nos. PCT/US2023/060850 and PCT/US2020/035752, which are each incorporated in their entirety by reference herein.

In several embodiments, genetic editing reduces transcription of CISH by about 30%, about 40%, about 50%, about 60%, about 70%, about 75%, about 80%, about 85%, about 90%, about 95%, about 97%, about 98%, about 99%, or more (including any amount between those listed). In several embodiments, genetic editing reduces transcription of CISH by at least about 30%. In several embodiments, genetic editing reduces transcription of CISH by at least about 40%. In several embodiments, genetic editing reduces transcription of CISH by at least about 50%. In several embodiments, genetic editing reduces transcription of CISH by at least about 60%. In several embodiments, genetic editing reduces transcription of CISH by at least about 70%. In several embodiments, genetic editing reduces transcription of CISH by at least about 80%. In several embodiments, genetic editing reduces transcription of CISH by at least about 90%. In several embodiments, genetic editing reduces transcription of CISH by at least about 95%. In several embodiments, genetic editing reduces transcription of CISH by at least about 99%.

In several embodiments, genetic editing can reduce expression of Cis by about 30%, about 40%, about 50%, about 60%, about 70%, about 75%, about 80%, about 85%, about 90%, about 95%, about 97%, about 98%, about 99%, or more (including any amount between those listed). In several embodiments, genetic editing reduces expression of Cis by about 30%, about 40%, about 50%, about 60%, about 70%, about 75%, about 80%, about 85%, about 90%, about 95%, about 97%, about 98%, about 99%, or more (including any amount between those listed). In several embodiments, genetic editing reduces expression of Cis by at least about 30%. In several embodiments, genetic editing reduces expression of Cis by at least about 40%. In several embodiments, genetic editing reduces expression of Cis by at least about 50%. In several embodiments, genetic editing reduces expression of Cis by at least about 60%. In several embodiments, genetic editing reduces expression of Cis by at least about 70%. In several embodiments, genetic editing reduces expression of Cis by at least about 80%. In several embodiments, genetic editing reduces expression of Cis by at least about 90%. In several embodiments, genetic editing reduces expression of Cis by at least about 95%. In several embodiments, genetic editing reduces expression of Cis by at least about 99%.

In some embodiments, immune cells (e.g., NK cells) are genetically edited to reduce or eliminate expression of an alternative or additional target gene, for example, transforming growth factor-beta receptor 2 protein (TGFbR2) and/or Casitas B-lineage lymphoma-b protein (Cbl-b). In some embodiments, the immune cells are genetically edited to reduce expression of TGFbR2. In some embodiments, the immune cells are genetically edited to reduce expression of Cbl-b. The expression of any other target protein, or any combination of target proteins, can be decreased or eliminated, such as by disrupting the gene(s) encoding the target protein(s).

### II. Compositions and Formulations

Also provided are compositions including immune cells (e.g., NK cells) genetically engineered to express a CD19-directed CAR, including pharmaceutical compositions and formulations. Also provided are compositions comprising genetically engineered NK cells that express any of the CD19-directed CARs described herein, including pharmaceutical compositions and formulations.

Provided are pharmaceutical formulations comprising genetically engineered NK cells expressing a CD19-directed CAR, a plurality of genetically engineered NK cells expressing a CD19-directed CAR, and/or additional agents for combination treatment or therapy. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell, binding molecule, and/or antibody, and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some aspects, a buffer is included in the composition. Suitable buffering agents include, for example, citric acid, sodium citrate, phosphoric acid, potassium phosphate, and various other acids and salts. In some aspects, a mixture of two or more buffers is used. The buffering agent or mixtures thereof are typically present in an amount of from about 0.001% to about 4% by weight of the total composition. Methods for preparing administrable pharmaceutical compositions are known.

Formulations of the antibodies described herein can include lyophilized formulations and aqueous solutions. The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., methotrexate or rituximab. In some embodiments, the cells or antibodies are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The pharmaceutical composition in some embodiments contains the engineered cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

Formulations include those for oral, intravenous, intraperitoneal, subcutaneous, pulmonary, transdermal, intramuscular, intranasal, buccal, sublingual, or suppository administration. In some embodiments, the agent or cell population is administered to the subject by intravenous, intraperitoneal, or subcutaneous injection using peripheral systemic delivery.

In some embodiments, the compositions are provided as sterile liquid formulations (e.g., isotonic aqueous solutions, suspensions, emulsions, dispersions, or viscous compositions), which in some aspects may be buffered to a selected pH. Liquid formulations are generally easier to prepare than gels, other viscous compositions, and solid compositions. In addition, liquid compositions are somewhat more convenient to administer, particularly by injection. The liquid composition can comprise a carrier, which can be a solvent or dispersion medium containing, for example, water, saline, phosphate buffered saline, polyols (e.g., glycerol, propylene glycol, liquid polyethylene glycol), and suitable mixtures thereof.

Sterile injectable solutions can be prepared by incorporating the agent or cell into a solvent, such as an admixture with a suitable carrier, diluent, or excipient (e.g., sterile water, saline, glucose, dextrose, and the like). Formulations for in vivo administration are typically sterile. Sterility can be readily achieved, for example, by filtration through sterile filtration membranes. In some embodiments, the dose of engineered cells administered is in a cryopreserved composition. In some aspects, the composition is administered after thawing the cryopreserved composition.

### III. Methods and Uses

Provided herein are methods of using and uses of the genetically engineered NK cells and pharmaceutical compositions and formulations thereof, such as in the treatment of an autoimmune disease (e.g., SLE), including any of those in which CD19 is implicated. Also provided herein are methods of using and uses of the genetically engineered NK cells and pharmaceutical compositions and formulations thereof, such as for the reduction of CD19-expressing cells (e.g., B cells) in a subject having a disease or condition, including any of those in which CD19 is implicated. Also provided herein are methods of using and uses of the genetically engineered NK cells and pharmaceutical compositions and formulations thereof, such as for reducing the level of an autoantibody in a subject having a B cell-mediated condition (e.g., an autoimmune disease).

In some aspects, also provided are methods of re-setting the B cell compartment (e.g., peripheral B cell compartment) in a subject having a B cell-mediated condition. In some aspects, re-setting of the B cell compartment is achieved when a majority of the repopulating B cells in a subject treated by a method provided herein are non-class-switched (e.g., IgM and/or IgD isotype) B cells. In some embodiments, greater than about 70%, greater than about 80%, greater than about 90%, or greater than about 95% of the repopulating B cells in a subject treated by a method provided herein are non-class-switched (e.g., IgM and/or IgD isotype). In some embodiments, greater than about 70% of the repopulating B cells in a subject treated by a method provided herein are non-class-switched (e.g., IgM and/or IgD isotype). In some embodiments, greater than about 80% of the repopulating B cells in a subject treated by a method provided herein are non-class-switched (e.g., IgM and/or IgD isotype). In some embodiments, greater than about 90% of the repopulating B cells in a subject treated by a method provided herein are non-class-switched (e.g., IgM and/or IgD isotype). In some embodiments, greater than about 95% of the repopulating B cells in a subject treated by a method provided herein are non-class-switched (e.g., IgM and/or IgD isotype). In some embodiments, the isotype of the repopulating B cells is assessed about 30 days, about 45 days, about 60 days, about 75 days, or about 90 days after administration of the last dose of a composition comprising immune cells expressing an anti-CD19 CAR. In some embodiments, the isotype of the repopulating B cells is assessed about 30 days after administration of the last dose of a composition comprising immune cells expressing an anti-CD19 CAR. In some embodiments, the isotype of the repopulating B cells is assessed about 45 days after administration of the last dose of a composition comprising immune cells expressing an anti-CD19 CAR. In some embodiments, the isotype of the repopulating B cells is assessed about 60 days after administration of the last dose of a composition comprising immune cells expressing an anti-CD19 CAR. In some embodiments, the isotype of the repopulating B cells is assessed about 75 days after administration of the last dose of a composition comprising immune cells expressing an anti-CD19 CAR. In some embodiments, the isotype of the repopulating B cells is assessed about 90 days after administration of the last dose of a composition comprising immune cells expressing an anti-CD19 CAR. In some embodiments, the repopulating B cells are repopulating peripheral B cells.

Among such methods, such as methods of treatment, and uses, are those that involve administering to a subject genetically engineered NK cells, such as a plurality of genetically engineered NK cells, expressing the provided anti-CD19 recombinant receptors (e.g. CARs). Such methods and uses include therapeutic methods and uses, for example, involving administration of the genetically engineered NK cells, or compositions containing the same, to a subject having a B cell-mediated disease, such as an autoimmune disease. Such methods and uses, thus, also include therapeutic methods and uses, for example, involving administration of the genetically engineered NK cells, or compositions containing the same, to a subject having an autoimmune disease (e.g., SLE). In some embodiments, the cell and/or composition is administered in an effective amount to effect treatment of the disease. Such methods and uses also include therapeutic methods and uses, for example, involving administration of the genetically engineered NK cells, or compositions containing the same, to a subject determined to be at risk, or at risk of relapse, of an autoimmune disease. In some embodiments, the subject is determined to be at risk of an autoimmune disease. Thus, in some embodiments, the composition is administered in an effective amount to effect prevention of the disease. In some embodiments, the subject is determined to be at risk of relapse of an autoimmune disease. Thus, in some embodiments, the composition is administered in an effective amount to effect prevention of relapse (e.g., recurrence) of the disease.

Also provided herein are uses of the genetically engineered NK cells in such methods and treatments, and in the preparation of a medicament in order to carry out such therapeutic methods. In some embodiments, the methods are carried out by administering the cells or compositions comprising the same, to the subject having, having had, or suspected of having a B cell-mediated disease (e.g., an autoimmune disease). Thus, in some embodiments, the methods are carried out by administering the cells or compositions comprising the same, to the subject having, having had, or suspected of having the autoimmune disease. In some embodiments, the methods or uses thereby treat the autoimmune disease in the subject. Also provided herein are of use of any of the compositions, such as pharmaceutical compositions provided herein, for the treatment of an autoimmune disease, such as use in a treatment regimen.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, alleviation of symptoms, diminishment of any direct or indirect pathological consequences of the disease, decreasing the rate of disease progression, amelioration or palliation of the disease state, and remission or improved prognosis. In some aspects, desirable effects of treatment include partial renal remission (PRR), complete renal remission (CRR), reduced number of flares and/or increased time between flares. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as SLE). This delay can be of varying lengths of time, depending on the history of the disease and/or subject being treated. In some embodiments, the provided molecules and compositions are used to delay development of a disease or to slow the progression of a disease. A sufficient or significant delay can, in effect, encompass prevention, in that the subject does not develop the disease.

“Preventing,” as used herein, includes providing prophylaxis with respect to the occurrence or recurrence of a disease in a subject that may be predisposed to the disease but has not yet been diagnosed with the disease.

As used herein, to “suppress” a function or activity is to reduce the function or activity when compared to otherwise same conditions except for a condition or parameter of interest, or alternatively, as compared to another condition.

An “effective amount” of an agent, e.g., a pharmaceutical formulation, binding molecule, antibody, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation, binding molecule, antibody, cells, or composition refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount. Alternatively, a prophylactic dose can be used in subjects to prevent a relapse (e.g., a recurrence such as a flare) of disease, in which case the prophylactically effective amount may be similar to, or the same as, a therapeutically effective amount.

As used herein, a “subject” or an “individual” is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject is human. In some embodiments, the subject is a human of at least 18 years of age. In some embodiments, the subject is a human of at least 12 years of age.

Methods for administration of cells for cell therapy are known and may be used in connection with the provided methods and compositions.

The disease or condition that is treated can be any in which expression of an antigen is associated with and/or involved in the etiology of a disease condition or disorder, e.g. causes, exacerbates or otherwise is involved in such disease, condition, or disorder. Non-limiting examples of diseases and conditions include B cell-mediated and/or autoimmune diseases. Non-limiting examples of antigens, which include antigens associated with various diseases and conditions that can be treated, include CD19. Among the diseases to be treated is any autoimmune disease in which CD19 is associated with and/or involved in the etiology of the disease.

In some embodiments, autoimmune diseases include, but are not limited to, lupus, including systemic lupus erythematosus (SLE), lupus nephritis (LN), and CNS lupus, inflammatory bowel disease (IBD, e.g. Crohn's disease or ulcerative colitis), rheumatoid arthritis (RA; e.g., juvenile rheumatoid arthritis), ANCA associated vasculitis, idiopathic thrombocytopenia purpura (ITP), thrombotic thrombocytopenia purpura (TTP), autoimmune thrombocytopenia, Chagas' disease. Grave's disease, Wegener's granulomatosis, polyarteritis nodosa, Sjogren's syndrome, pemphigus vulgaris, scleroderma, multiple sclerosis (MS), psoriasis, IgA nephropathy, IgM polyneuropathies, vasculitis, diabetes mellitus, Reynaud's syndrome, anti-phospholipid syndrome, Goodpasture's disease, Kawasaki disease, autoimmune hemolytic anemia, myasthenia gravis (MG), or progressive glomerulonephritis. In some embodiments, a subject may exhibit symptoms of more than one autoimmune disease. In some embodiments, the autoimmune disease is systemic lupus erythematosus (SLE). In some embodiments, the autoimmune disease is SLE with renal involvement (e.g., lupus nephritis (LN)). In some embodiments, the autoimmune disease is lupus nephritis (LN). In some embodiments, the autoimmune disease is CNS lupus. In some embodiments, the autoimmune disease is IBD. In some embodiments, the autoimmune disease is Crohn's disease. In some embodiments, the autoimmune disease is ulcerative colitis. In some embodiments, the autoimmune disease is vasculitis. In some embodiments, the autoimmune disease is ANCA vasculitis (AAV). In some embodiments, the autoimmune disease is autoimmune encephalitis (AE). In some embodiments, the autoimmune disease is ITP. In some embodiments, the autoimmune disease is TTP. In some embodiments, the autoimmune disease is autoimmune thrombocytopenia. In some embodiments, the autoimmune disease is Chagas' disease. In some embodiments, the autoimmune disease is Graves' disease. In some embodiments, the autoimmune disease is Wegener's granulomatosis. In some embodiments, the autoimmune disease is polyarteritis nodosa. In some embodiments, the autoimmune disease is Sjogren's syndrome. In some embodiments, the autoimmune disease is pemphigus vulgaris. In some embodiments, the autoimmune disease is psoriasis. In some embodiments, the autoimmune disease is IgA nephropathy. In some embodiments, the autoimmune disease is membranous nephropathy (MN). In some embodiments, the autoimmune disease is IgM polyneuropathies. In some embodiments, the autoimmune disease is vasculitis. In some embodiments, the autoimmune disease is diabetes mellitus. In some embodiments, the autoimmune disease is Reynaud's syndrome. In some embodiments, the autoimmune disease is anti-phospholipid syndrome. In some embodiments, the autoimmune disease is Goodpasture's disease. In some embodiments, the autoimmune disease is Kawasaki disease. In some embodiments, the autoimmune disease is autoimmune hemolytic anemia. In some embodiments, the autoimmune disease is myasthenia gravis (MG). In some embodiments, the autoimmune disease is progressive glomerulonephritis. In some embodiments, the autoimmune disease is acquired immunodeficiency syndrome (AIDS). In some embodiments, the autoimmune disease is Addison's disease. In some embodiments, the autoimmune disease is alopecia areata. In some embodiments, the autoimmune disease is celiac disease. In some embodiments, the autoimmune disease is chronic inflammatory demyelinating polyneuropathy (CIDP). In some embodiments, the autoimmune disease is Guillain-Barre syndrome. In some embodiments, the autoimmune disease is Hashimoto thyroiditis. In some embodiments, the autoimmune disease is pernicious anemia. In some embodiments, the autoimmune disease is psoriasis. In some embodiments, the autoimmune disease is psoriatic arthritis. In some embodiments, the autoimmune disease is reactive arthritis. In some embodiments, the autoimmune disease is rheumatoid arthritis (RA). In some embodiments, the autoimmune disease is refractory RA. In some embodiments, RA is refractory to a TNF inhibitor. In some embodiments, the autoimmune disease is multiple sclerosis (MS). In some embodiments, MS is primary progressive MS (PPMS). In some embodiments. MS is secondary-progressive MS (SPMS). In some embodiments, MS is relapsing-remitting MS (RRMS). In some embodiments, the subject does not have antiphospholipid syndrome.

In some embodiments, the autoimmune disease comprises scleroderma. In some embodiments, the autoimmune disease is scleroderma. In some embodiments, the autoimmune disease comprises systemic sclerosis (also known as systemic scleroderma). In some embodiments, the autoimmune disease is systemic sclerosis (also known as systemic scleroderma). In some embodiments, the autoimmune disease comprises localized scleroderma. In some embodiments, the autoimmune disease is localized scleroderma.

In some embodiments, the autoimmune disease comprises myositis (also known as IIM). In some embodiments, the autoimmune disease is myositis (also known as IIM). In some embodiments, the autoimmune disease is selected from the group consisting of anti-synthetase syndrome (ASSD), overlap myopathy (OM), dermatomyositis (DM), clinically amyopathic dermatomyositis, juvenile myositis (JM), necrotizing myopathy (NM; e.g., necrotizing autoimmune myopathy (or immune-mediated necrotizing myopathy), polymyositis (PM), and sporadic inclusion body myositis (sIBM). In some embodiments, the autoimmune disease is ASSD. In some embodiments, the autoimmune disease is OM. In some embodiments, the autoimmune disease is DM. In some embodiments, the autoimmune disease is JM. In some embodiments, the autoimmune disease is NM. In some embodiments, the autoimmune disease is PM. In some embodiments, the autoimmune disease is sIBM.

In some embodiments, the autoimmune disease comprises vasculitis. In some embodiments, the autoimmune disease is vasculitis. In some embodiments, the vasculitis is large-vessel vasculitis. In some embodiments, the vasculitis is medium-vessel vasculitis. In some embodiments, the vasculitis is small-vessel vasculitis. In some embodiments, the vasculitis is anti-neutrophilic cytoplasmic autoantibody (ANCA) vasculitis. In some embodiments the ANCA vasculitis is granulomatosis with polyangiitis (GPA). In some embodiments the ANCA vasculitis is microscopic polyangiitis (MPA). In some embodiments the ANCA vasculitis is eosinophilic granulomatosis with polyangiitis (EGPA).

In some embodiments, the autoimmune disease comprises myasthenia gravis (MG). In some embodiments, the autoimmune disease is MG. In some embodiments, MG is ocular MG. In some embodiments, MG is early-onset generalized MG. In some embodiments, MG is late-onset MG.

In some embodiments, the autoimmune disease is autoimmune encephalitis (AE). In some embodiments, AE comprises an antibody to an intracellular antigen (e.g., anti-Hu or anti-GAD65). In some embodiments, AE comprises an autoantibody to an extracellular epitope of an ion channel, receptor, and/or other associated protein (e.g., anti-NMDA receptor).

In some embodiments, the subject has persistent or relapsed disease, e.g., following treatment with a B cell-targeting agent (e.g., an anti-BAFF, anti-CD19, or anti-CD20 antibody). In some embodiments, the administration effectively treats the subject despite the subject having become resistant to a previous B cell-targeting agent. In some embodiments, the subject has not relapsed but is determined to be at risk for relapse, such as at a high risk of relapse, and thus the composition is administered prophylactically, e.g., to reduce the likelihood of or prevent relapse.

In some embodiments, prior to the initiation of administration of the genetically engineered NK cells, the subject has received one or more prior therapies. In some embodiments, the subject has received at least 1, 2, 3, 4, 5, 6, 7, 8, 9, 10, 11, 12, 13, 14, 15, 16, 17, 18, 19 or 20 or more prior therapies. In some embodiments, the subject has received at least 3, 4, 5, 6, 7, 8, 9, 10 or more prior therapies. In some embodiments, the subject has received at least 1 prior therapy. In some embodiments, the subject has received at least 2 prior therapies. In some embodiments, the subject has received at least 3 prior therapies. In some embodiments, the subject has received at least 4 prior therapies.

In some aspects, the subject has relapsed or has been refractory to the one or more prior therapies. In some aspects, the prior therapies include treatment with a B cell-targeting agent (e.g., an anti-BAFF, anti-CD19, anti-CD20, or anti-CD22 antibody), an immunosuppressive agent, a steroid (e.g., a corticosteroid), a nonsteroidal anti-inflammatory drug (NSAID), an antimalarial agent, or HSCT. In some aspects, the prior therapies include treatment with a B cell-targeting agent. In some aspects, the one or more prior therapies comprises an immunosuppressive agent. In some aspects, the one or more prior therapies comprises a steroid (e.g., a corticosteroid). In some aspects, the one or more prior therapies comprises a nonsteroidal anti-inflammatory drug (NSAID). In some aspects, the one or more prior therapies comprises an antimalarial agent. In some aspects, the one or more prior therapies comprises HSCT.

In some embodiments, the cell therapy is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. Thus, in some embodiments, the cells are allogeneic to the subject to be treated.

In some embodiments, the subject, to whom the cells, cell populations, or compositions are administered, is a primate, such as a human. In some embodiments, the subject, to whom the cells, cell populations, or compositions are administered, is a non-human primate. In some embodiments, the non-human primate is a monkey (e.g., cynomolgus monkey) or an ape. In some embodiments, the subject is a non-primate mammal, such as a rodent (e.g., mouse, rat, etc.). The subject can be male or female and can be any suitable age, including infant, juvenile, adolescent, adult, and geriatric subjects. In some embodiments, the subject is a pediatric subject (e.g., a juvenile or adolescent). For example, in some embodiments, the subject is less than 18 years of age. In some embodiments, the subject is between about 12 years of age and about 18 years of age. In some embodiments, the subject is at least 12 years of age. In some embodiments, the subject is between about 14 years of age and about 18 years of age. In some embodiments, the subject is at least 14 years of age. In some embodiments, the subject is between about 16 years of age and about 18 years of age. In some embodiments, the subject is at least 16 years of age. It is contemplated that pediatric subjects may have worse outcomes, more severe disease, and/or lower compliance with treatment regimens, as compared to adult subjects (e.g., subjects at least 18 years of age).

In some embodiments, the subject is an adult. In some embodiments, the subject is between 18 and 65 years of age. In some embodiments, the subject is a human of at least 18 years of age.

In some embodiments, a subject is selected for treatment based on a disease activity index (e.g., the subject's score on a disease activity index). In some aspects, the method includes selecting a subject for treatment based on a disease activity index (e.g., the subject's score on a disease activity index). The disease activity index can be any of those described in this Section III.

In some embodiments, the subject does not have CNS involvement, such as neuropsychiatric systemic lupus erythematosus (NPSLE) (also known as CNS lupus). In some embodiments, the subject does not have active CNS lupus at the time of administration of the genetically engineered NK cell composition. In some embodiments, the subject has not had active CNS lupus within one year prior to administration of the genetically engineered NK cell composition. In some embodiments, active CNS lupus comprises aseptic meningitis, ataxia, CNS vasculitis, cranial neuropathy, demyelinating syndrome, optic neuritis, psychosis, seizures, transverse myelitis, or any combination thereof. In some embodiments, the subject has CNS involvement, such as NPSLE.

In some embodiments, the subject does not have lupus nephritis (LN). In some embodiments, the subject has SLE with renal involvement (e.g., LN). In some embodiments, the subject has Class III or Class IV LN, e.g., as defined by the History of International Society of Nephrology/Rental Pathology Society (ISN/RPS). Bajema et al., Kidney Int. 2018; 93 (4): 789-96. In some embodiments, the subject has Class III or Class IV LN within about 6 months or within about 7 months prior to administration of the genetically engineered NK cell composition. In some embodiments, the subject has Class III or Class IV LN (with or without Class V LN). In some embodiments, the subject has Class III LN. In some embodiments, the subject has Class IV LN. In some embodiments, the subject does not have Class V LN. In some embodiments, the subject has Class V LN. In some embodiments, LN comprises a urine protein creatinine ratio of greater than or equal to 2000 mg/g (or equivalent) at the time of administration of the genetically engineered NK cell composition to the subject. In some embodiments, active LN is defined as proteinuria greater than or equal to 1500 mg/24 hours when assessed by 24-hour urine collection, e.g., as defined by greater than or equal to 1.5 mg/mg urine protein creatinine ratio (UPCR). In some embodiments, active LN is defined as urinary protein: creatinine ratio (UPCR) of greater than or equal to 7.0 g/g or proteinuria greater than or equal to 1.5 g/day. In some embodiments, the subject has not required induction therapy for the autoimmune disease (e.g., SLE) within about one year prior to administration of the genetically engineered NK cell composition. In some embodiments, the subject does not have histological evidence of diffuse proliferative glomerulonephritis within about 12 weeks prior to administration of the genetically engineered NK cell composition. In some embodiments, the subject has lupus nephritis (LN).

In some embodiments, at the time of administration of the engineered NK cell composition, the subject has a score of 10 or more points on the European League Against Rheumatism (EULAR)/American College of Rheumatology (ACR) 2019 classification criteria for SLE. Aringer et al., Arthritis Rheumatol (2019) 71 (9): 1400-12. In some embodiments, at the time of administration of the engineered NK cell composition, the subject has a score of 10 or more points on the 2012 SLICC criteria for SL. Petri et al., Arthritis Rheum (2012) 64:2677-86. In some embodiments, at the time of administration of the engineered NK cell composition, the subject has a Systemic Lupus Erythematosus Disease Activity Index 2000 (SLEDAI-2K) score of 4 or greater. In some embodiments, at the time of administration of the engineered NK cell composition, the subject has a Systemic Lupus Erythematosus Disease Activity Index 2000 (SLEDAI-2K) score of 6 or greater. Gladman et al., J Rheumatol (2002) 29 (2): 288-91. In some embodiments, at the time of administration of the engineered NK cell composition, the subject is positive for one or more of antinuclear antibodies (ANA), anti-double stranded DNA (anti-dsDNA) antibodies, and anti-Smith (anti-Sm) antibodies. In some embodiments, at the time of administration of the engineered NK cell composition, the subject is positive for ANA. In some embodiments, positivity for ANA is defined by an ANA titer of greater than or equal to 1:80 in a sample (e.g., serum sample) from the subject. In some embodiments, at the time of administration of the engineered NK cell composition, the subject is positive for anti-dsDNA antibodies. In some embodiments, at the time of administration of the engineered NK cell composition, the subject is positive for anti-Sm antibodies. Methods for determining whether a subject is positive for ANA, anti-dsDNA antibodies, and anti-Sm antibodies are known and described in the art.

In some embodiments, the subject has multiple sclerosis (MS), e.g. as defined by the 2017 McDonald diagnostic criteria (Thompson et al., Lancet Neurol. (2018) 17:162-73). In some embodiments, the MS is RRMS. In some embodiments, the MS is SPMS. In some embodiments, the MS is PPMS. In some embodiments, the subject has a one-year history of disability progression and any two of: (1) one or more T2 lesions characteristic of MS in one or more typical brain regions (periventricular, cortical or juxtacortical, infratentorial); (2) two or more T2 lesions in the spinal cord, and (3) the presence of CSF-specific oligoclonal bands. In some embodiments, the prior therapies comprise an anti-CD20 antibody (e.g., ocrelizumab or rituximab). In some embodiments, the prior therapies comprise a selective sphingosine-1-phosphate receptor 1 and 5 modulator (e.g., siponimod). In some embodiments, the prior therapies comprise a DNA intercalating agent (e.g., mitoxantrone). In some embodiments, the prior therapies comprise a purine analog (e.g., cladribine).

In some embodiments, the subject has myasthenia gravis (MG). In some embodiments, the MG is ocular MG. In some embodiments, the MG is generalized MG. In some embodiments, the subject has a score of 4 or greater on MG-ADL. In some embodiments, the subject has a score of 6 or greater on MG-ADL. In some embodiments, the subject has a score of 8 or greater on MG-ADL. In some embodiments, the subject has a score of 10 or greater on MG-ADL. In some embodiments, the subject has MGFA Class II, III, or IV. In some embodiments, the subject has MGFA Class II. In some embodiments, the subject has MGFA Class III. In some embodiments, the subject has MGFA Class IV. In some embodiments, the subject has anti-AChR antibodies. In some embodiments, the subject has anti-MuSK antibodies. In some embodiments, the subject has anti-LRP4 antibodies. In some embodiments, the prior therapies comprise a thymectomy. In some embodiments, the prior therapies comprise an anti-CD20 antibody (e.g., rituximab). In some embodiments, the prior therapies comprise an antimetabolite (e.g., methotrexate). In some embodiments, the prior therapies comprise an anti-complement antibody (e.g., an anti-C5 antibody). In some embodiments, the prior therapies comprise an immune checkpoint inhibitor (e.g., an anti-PD1, -PDL1, or CTLA antibody). In some embodiments, the prior therapies comprise an AchE inhibitor (e.g., edrophonium chloride).

In some embodiments, the subject has IIM (also known as myositis). In some embodiments, the subject has a diagnosis of probably or definite (>55%) idiopathic inflammatory myopathy according to the 2017 ACR/EULAR Classification Criteria (Lundberg. Tjarnlund et al. 2017). In some embodiments, the subject has a MMT score of ≤80. In some embodiments, the myositis is antisynthetase syndrome. In some embodiments, the myositis is dermatomyositis. In some embodiments, the subject has a skin rash. In some embodiments, the subject has proximal upper and lower limb weakness. In some embodiments, the subject has anti-SAE antibodies. In some embodiments, the subject has anti-Mi2 antibodies. In some embodiments, the subject has anti-MDA5 antibodies. In some embodiments, the subject has anti-NXP2 antibodies. In some embodiments, the subject has anti-TIF1 antibodies. In some embodiments, the myositis is juvenile myositis. In some embodiments, the myositis is necrotizing myopathy. In some embodiments, the subject has anti-SRP antibodies. In some embodiments, the subject has anti-HMGCR antibodies. In some embodiments, the myositis is polymyositis. In some embodiments, the subject has anti-PM-Scl antibodies. In some embodiments, the subject has anti-Scl-70 antibodies. In some embodiments, the subject has anti-Ku antibodies. In some embodiments, the subject has anti-RNP antibodies. In some embodiments, the subject has anti-Ro/SSA or anti-La/SSB antibodies. In some embodiments, the myositis is sporadic inclusion body myositis (sIBM). In some embodiments, the subject has anti-CN1A antibodies. In some embodiments, the prior therapies comprise intravenous immunoglobulin (IVIg). In some embodiments, the prior therapies comprise a costimulation modulator (e.g., abatacept).

In some embodiments, the subject has scleroderma. In some embodiments, the scleroderma is systemic scleroderma (also known as systemic sclerosis). In some embodiments, the subject is classified as having systemic sclerosis according to 2013 ACR/EULAR Classification Criteria with a total score of ≥9. In some embodiments, the subject has active disease. In some embodiments, active disease is defined as a MRSS score of ≥15. In some embodiments, the subject has anti-centromere antibodies. In some embodiments, the subject has anti-topoisomerase 1 antibodies. In some embodiments, the subject has anti-RNA polymerase III antibodies. In some embodiments, the scleroderma is localized scleroderma. In some embodiments, the prior therapies comprise an immunosuppressive therapy (e.g., methotrexate, cyclophosphamide, mycophenolate mofetil, cyclosporine, azathioprine). In some embodiments, the prior therapies comprise a calcium channel blocker. In some embodiments, the prior therapies comprise an endothelin receptor antagonist. In some embodiments, the prior therapies comprise a PDE5 inhibitor. In some embodiments, the prior therapies comprise one or more of prokinetic agents, proton pump inhibitors, ACE inhibitors, anticoagulants, prostacyclins, phototherapy, and steroids (e.g., corticosteroids).

In some embodiments, the subject has vasculitis. In some embodiments, the subject has anti-neutrophil cytoplasmic antibodies (ANCA). In some embodiments, the subject has ANCA-associated vasculitis (AAV). In some embodiments, the AAV is GPA. In some embodiments, the AAV is MPA. In some embodiments, the subject has renal limited vasculitis. In some embodiments, the AAV is EGPA. In some embodiments, the subject has anti-proteinase-3 (PR3-ANCA). In some embodiments, the subject has anti-myeloperoxidase (MPO-ANCA). In some embodiments, the prior therapies comprise steroids (e.g., glucocorticoids). In some embodiments, the prior therapies comprise an immunosuppressive agent (e.g., cyclophosphamide, methotrexate, mycophenolate mofetil). In some embodiments, the prior therapies comprise an anti-CD20 antibody (e.g., rituximab). In some embodiments, the prior therapies comprise IVIg.

In some embodiments, the presence or level of an antibody is determined by biopsy, e.g., analysis of a muscular biopsy. In some embodiments, the presence or level of the antibody is determined in a blood sample. In some embodiments, if the antibody is present in a blood sample from the subject, the subject is said to be seropositive for the antibody.

In some embodiments, the subject was diagnosed with the autoimmune disease (e.g., SLE or LN) between at least about 18 weeks and at least about 30 weeks prior to administration of the composition. In some embodiments, the subject was diagnosed with the autoimmune disease at least about 18 weeks, at least about 20 weeks, at least about 22 weeks, at least about 24 weeks, at least about 26 weeks, at least about 28 weeks, or at least about 30 weeks prior to administration of the composition. In some embodiments, the subject was diagnosed with the autoimmune disease between at least about 20 weeks prior to administration of the composition. In some embodiments, the subject was diagnosed with the autoimmune disease between at least about 21 weeks prior to administration of the composition. In some embodiments, the subject was diagnosed with the autoimmune disease between at least about 22 weeks prior to administration of the composition. In some embodiments, the subject was diagnosed with the autoimmune disease between at least about 23 weeks prior to administration of the composition. In some embodiments, the subject was diagnosed with the autoimmune disease between at least about 24 weeks prior to administration of the composition. In some embodiments, the subject was diagnosed with the autoimmune disease between at least about 25 weeks prior to administration of the composition. In some embodiments, the subject was diagnosed with the autoimmune disease between at least about 26 weeks prior to administration of the composition. In some embodiments, the subject was diagnosed with the autoimmune disease between at least about 27 weeks prior to administration of the composition. In some embodiments, the subject was diagnosed with the autoimmune disease between at least about 28 weeks prior to administration of the composition. In some embodiments, the subject was diagnosed with the autoimmune disease between at least about 29 weeks prior to administration of the composition. In some embodiments, the subject was diagnosed with the autoimmune disease between at least about 30 weeks prior to administration of the composition. In some embodiments, the subject has had SLE for at least about 6 months prior to administration of the engineered NK cell composition.

In some embodiments, the subject has not been previously treated with CAR T cells (e.g., CD19 CAR T cells). Thus, in some embodiments, the subject is CAR T cell (e.g., CD19 CAR T cell) naïve. In some embodiments, the subject has been previously treated with CAR T cells (e.g., CD19 CAR T cells). Thus, in some embodiments, the subject is CAR T cell (e.g., CD19 CAR T cell) exposed. In some embodiments, the subject is relapsed/refractory to CAR T cells (e.g., CD19 CAR T cells). In some embodiments, the CAR T cells are CD19 CAR T cells.

In some embodiments, the subject has not been previously treated with CAR NK cells (e.g., CD19 CAR NK cells). Thus, in some embodiments, the subject is CAR NK cell (e.g., CD19 CAR NK cell) naïve. In some embodiments, the subject has been previously treated with CAR NK cells (e.g., CD19 CAR NK cells). Thus, in some embodiments, the subject is CAR NK cell (e.g., CD19 CAR NK cell) exposed. In some embodiments, the subject is relapsed/refractory to CAR NK cells (e.g., CD19 CAR NK cells). In some embodiments, the CAR NK cells are CD19 CAR NK cells.

In some embodiments, the subject has not been previously treated with a CD19-directed therapy. In some embodiments, the subject has not been previously treated with a mesenchymal cell therapy. In some embodiments, the subject has not had a solid organ transplant. In some embodiments, the subject has not had a hematopoietic cell transplant.

The genetically engineered cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells.

In some embodiments, administration of the genetically engineered cell composition or any additional therapies, e.g., a lymphodepleting therapy and/or combination therapy, is carried out via outpatient delivery. In some embodiments, administration of the genetically engineered cell composition is carried out via outpatient delivery. In some embodiments, administration of the first dosing cycle is carried out via outpatient delivery. In some embodiments, administration of a subsequent dosing cycle is carried out via outpatient delivery. In some embodiments, administration of each dosing cycle is carried out via outpatient delivery. For the prevention or treatment of disease, the appropriate dosage of the cell or composition containing the same may depend on the type of disease to be treated, the type of binding molecule or recombinant receptor, the severity and course of the disease, whether the binding molecule or recombinant receptor is administered for preventive or therapeutic purposes, previous therapy, the patient's clinical history and response to the recombinant receptor or cell, and the discretion of the attending physician.

In some embodiments, the dose and/or frequency of administration is determined based on efficacy and/or response. In some embodiments efficacy and/or response is determined by any measure known in the art, including those described in Arora et al., Arthritis Care Res (2020) 72 (S10): 27-46. In some examples, dose and/or frequency of administration is determined by the expansion and persistence of the recombinant receptor or cell in the blood and/or bone marrow. In some embodiments, dose and/or frequency of administration is determined based on the presence of autoantibodies in a sample from a subject.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; and (xi) an enhancement, improvement, supplementation, complementation, or augmentation of the prophylactic or therapeutic effect(s) of another therapy.

Non-limiting examples of the ability of anti-CD19 CAR expressing NK cells as provided herein to exert cytotoxicity against CD19-expressing target cells is described in Morisot et al., J ImmunoTher Canc (2020) 8 (suppl. 3): Abstract 127 and PCT Application Nos. PCT/US2020/020824 and PCT/US2023/069403, each of which are incorporated herein by reference in their entirety.

In some embodiments, the methods comprise administering a dose of the engineered cells or a composition comprising the engineered cells. In some embodiments, the methods comprise administering one, two, or three doses of the engineered cells or a composition comprising the engineered cells. In some embodiments, the engineered cells or compositions containing engineered cells can be used in a treatment regimen, wherein the treatment regimen (e.g., a dosing cycle) comprises administering a dose of the engineered cells or a composition comprising the engineered cells. In some embodiments, the engineered cells or compositions containing engineered cells can be used in a treatment regimen, wherein the treatment regimen comprises administering one, two, or three doses of the engineered cells or a composition comprising the engineered cells. In some embodiments, the dose can contain, for example, a particular number or range of recombinant receptor-expressing immune cells (e.g., NK cells), such as any number of such cells described herein. In some embodiments, a composition containing a dose of the cells can be administered. In some aspects, the number, amount or proportion of CAR-expressing cells in a cell population or a cell composition can be assessed by detection of a surrogate marker, e.g., by flow cytometry or other means, or by detecting binding of a labelled molecule, such as a labelled antigen, that can specifically bind to the binding molecules or receptors provided herein.

Doses of immune cells such as NK cells can be determined for a given subject based on their body mass, disease type and state, and desired aggressiveness of treatment, but range, depending on the embodiments, from about 10⁵ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of NK cells is administered, for example between about 1×10⁶ cells/kg to about 1×10⁸ cells/kg.

In some embodiments, each dose of the dosing cycle comprises between about 1×10⁶ CAR-expressing NK cells/kilogram (kg) and about 1×10⁸ CAR-expressing NK cells/kg. In some embodiments, if the subject weighs less than 50 kilograms, each dose of the dosing cycle comprises between about 1×10⁶ CAR-expressing NK cells/kg and about 1×10⁸ CAR-expressing NK cells/kg. In some embodiments, each dose of the dosing cycle comprises about 1×10⁶ CAR-expressing NK cells/kg. In some embodiments, each dose of the dosing cycle comprises about 2×10⁶ CAR-expressing NK cells/kg. In some embodiments, each dose of the dosing cycle comprises about 3×10⁶ CAR-expressing NK cells/kg. In some embodiments, each dose of the dosing cycle comprises about 4×10⁶ CAR-expressing NK cells/kg. In some embodiments, each dose of the dosing cycle comprises about 5×10⁶ CAR-expressing NK cells/kg. In some embodiments, each dose of the dosing cycle comprises about 6×10⁶ CAR-expressing NK cells/kg. In some embodiments, each dose of the dosing cycle comprises about 7×10⁶ CAR-expressing NK cells/kg. In some embodiments, each dose of the dosing cycle comprises about 8×10⁶ CAR-expressing NK cells/kg. In some embodiments, each dose of the dosing cycle comprises about 9×10⁶ CAR-expressing NK cells/kg. In some embodiments, each dose of the dosing cycle comprises about 1×10⁷ CAR-expressing NK cells/kg. In some embodiments, each dose of the dosing cycle comprises about 2×10⁷ CAR-expressing NK cells/kg. In some embodiments, each dose of the dosing cycle comprises about 3×10⁷ CAR-expressing NK cells/kg. In some embodiments, each dose of the dosing cycle comprises about 4×10⁷ CAR-expressing NK cells/kg. In some embodiments, each dose of the dosing cycle comprises about 5×10⁷ CAR-expressing NK cells/kg. In some embodiments, each dose of the dosing cycle comprises about 6×10⁷ CAR-expressing NK cells/kg. In some embodiments, each dose of the dosing cycle comprises about 7×10⁷ CAR-expressing NK cells/kg. In some embodiments, each dose of the dosing cycle comprises about 8×10⁷ CAR-expressing NK cells/kg. In some embodiments, each dose of the dosing cycle comprises about 9×10⁷ CAR-expressing NK cells/kg. In some embodiments, each dose of the dosing cycle comprises about 1×10⁸ CAR-expressing NK cells/kg.

In some embodiments, a dose of engineered cells comprises between about 300×10⁶ and 3×10⁹ NK cells. In some embodiments, a dose of engineered cells comprises about 300×10⁶ NK cells. In some embodiments, a dose of engineered cells comprises about 500×10⁶ NK cells. In some embodiments, a dose of engineered cells comprises about 1×10⁹ NK cells. In some embodiments, a dose of engineered cells comprises about 1.25×10⁹ NK cells. In some embodiments, a dose of engineered cells comprises about 1.5×10⁹ NK cells. In some embodiments, a dose of engineered cells comprises about 1.75×10⁹ NK cells. In some embodiments, a dose of engineered cells comprises about 2×10⁹ NK cells. In some embodiments, a dose of engineered cells comprises about 2.5×10⁹ NK cells. In some embodiments, a dose of engineered cells comprises about 3×10⁹ NK cells.

In some embodiments, a dose of engineered cells comprises between about 3×10⁸ and 5×10⁹ CAR-expressing NK cells. In some embodiments, a dose of engineered cells comprises between about 1×10⁹ and 3×10⁹ CAR-expressing NK cells. In some embodiments, a dose of engineered cells comprises about 3×10⁸ CAR-expressing NK cells. In some embodiments, a dose of engineered cells comprises about 5×10⁸ CAR-expressing NK cells. In some embodiments, a dose of engineered cells comprises about 1×10⁹ CAR-expressing NK cells. In some embodiments, a dose of engineered cells comprises about 1.25×10⁹ CAR-expressing NK cells. In some embodiments, a dose of engineered cells comprises about 1.5×10⁹CAR-expressing NK cells. In some embodiments, a dose of engineered cells comprises about 1.75×10⁹ CAR-expressing NK cells. In some embodiments, a dose of engineered cells comprises about 2×10⁹ CAR-expressing NK cells. In some embodiments, a dose of engineered cells comprises about 2.5×10⁹ CAR-expressing NK cells. In some embodiments, a dose of engineered cells comprises about 3×10⁹ CAR-expressing NK cells. In some embodiments, a dose of engineered cells comprises about 3.5×10⁹ CAR-expressing NK cells. In some embodiments, a dose of engineered cells comprises about 4×10⁹ CAR-expressing NK cells. In some embodiments, a dose of engineered cells comprises about 4.5×10⁹ CAR-expressing NK cells. In some embodiments, a dose of engineered cells comprises about 5×10⁹ CAR-expressing NK cells.

In several embodiments, multiple doses are used, for example, two, three, four, or more doses within a dosing cycle. In some embodiments, a dosing cycle consists of two doses. In some embodiments, a dosing cycle consists of three doses. In some embodiments, a dosing cycle consists of four doses. In some embodiments, a dosing cycle consists of five doses. Such multi-dose cycles can be repeated one or more times, as needed to treat an autoimmune disease or disease progression.

In some embodiments, all doses of the dosing cycle are administered to the subject within about 10 days, within about 9 days, within about 8 days, within about 7 days, within about 6 days, within about 5 days, or within about 4 days. In some embodiments, all doses of the dosing cycle are administered to the subject within about 10 days. In some embodiments, all doses of the dosing cycle are administered to the subject within about 9 days. In some embodiments, all doses of the dosing cycle are administered to the subject within about 8 days. In some embodiments, all doses of the dosing cycle are administered to the subject within about 7 days. In some embodiments, all doses of the dosing cycle are administered to the subject within about 6 days. In some embodiments, all doses of the dosing cycle are administered to the subject within about 5 days. In some embodiments, all doses of the dosing cycle are administered to the subject within about 4 days. In some embodiments, all doses of a dosing cycle are administered to the subject within about 13 days, within about 12 days, within about 11 days, within about 10 days, within about 9 days, within about 8 days, or within about 7 days of administration of a lymphodepleting therapy to a subject. In some embodiments, all doses of a dosing cycle are administered to the subject within about 13 days of a lymphodepleting therapy to a subject. In some embodiments, all doses of a dosing cycle are administered to the subject within about 12 days of a lymphodepleting therapy to a subject. In some embodiments, all doses of a dosing cycle are administered to the subject within about 11 days of a lymphodepleting therapy to a subject. In some embodiments, all doses of a dosing cycle are administered to the subject within about 10 days of a lymphodepleting therapy to a subject. In some embodiments, all doses of a dosing cycle are administered to the subject within about 9 days of a lymphodepleting therapy to a subject. In some embodiments, all doses of a dosing cycle are administered to the subject within about 8 days of a lymphodepleting therapy to a subject. In some embodiments, all doses of a dosing cycle are administered to the subject within about 7 days of a lymphodepleting therapy to a subject.

In some embodiments, the dosing cycle consists of three doses. In some embodiments, the lymphodepleting therapy does not comprise fludarabine. In some embodiments, the lymphodepleting therapy consists of cyclophosphamide.

In several embodiments, dosing is, for example, 3 doses of between about 0.5×10⁹ NK cells and about 2.5×10⁹ NK cells administered over about 21 to 28 days. In several embodiments, dosing is, for example, 3 doses of about 1.0×10⁹ NK cells or about 1.5×10⁹ NK cells administered over about 21 to 28 days. In several embodiments, a dosing cycle comprises 3 doses of about 1×10⁸ NK cells administered over about 21 to 28 days. In several embodiments, a dosing cycle comprises 3 doses of about 3×10⁸ NK cells administered over about 21 to 28 days. In several embodiments, a dosing cycle comprises 3 doses of about 1.0×10⁹ CAR-expressing NK cells administered over about 21 to 28 days. In several embodiments, a dosing cycle comprises 3 doses of about 1.5×10⁹ NK cells administered over about 21 to 28 days. In several embodiments, a dosing cycle comprises 3 doses of about 2.0×10⁹ NK cells administered over about 21 to 28 days. In several embodiments, a dosing cycle comprises 3 doses of about 2.5×10⁹ CAR-expressing NK cells administered over about 21 to 28 days.

In several embodiments, a dosing cycle comprises 3 doses. In some embodiments, a dosing cycle is about 28 days. In several embodiments, dosing is, for example, 3 doses of between about 3×10⁸ CAR-expressing NK cells and about 5×10⁹ CAR-expressing NK cells administered over a dosing cycle of about 28 days. In several embodiments, dosing is, for example, 3 doses of about 3×10⁸ CAR-expressing NK cells, about 1×10⁹ CAR-expressing NK cells, about 1.5×10⁹ CAR-expressing NK cells, about 2×10⁹ CAR-expressing NK cells, about 2.5×10⁹ CAR-expressing NK cells, about 3×10⁹ CAR-expressing NK cells, about 3.5×10⁹ CAR-expressing NK cells, about 4×10⁹ CAR-expressing NK cells, about 4.5×10⁹ CAR-expressing NK cells, or about 5×10⁹ CAR-expressing NK cells administered over about 28 days. In several embodiments, a dosing cycle comprises 3 doses of about 3×10⁸ NK cells administered over about 28 days. In several embodiments, a dosing cycle comprises 3 doses of about 5×10⁸ NK cells administered over about 28 days. In several embodiments, a dosing cycle comprises 3 doses of about 1×10⁹ NK cells administered over about 28 days. In several embodiments, a dosing cycle comprises 3 doses of about 1.5×10⁹ NK cells administered over about 28 days. In several embodiments, a dosing cycle comprises 3 doses of about 2×10⁹ NK cells administered over about 28 days. In several embodiments, a dosing cycle comprises 3 doses of about 2.5×10⁹ NK cells administered over about 28 days. In several embodiments, a dosing cycle comprises 3 doses of about 3×10⁹ NK cells administered over about 28 days. In several embodiments, a dosing cycle comprises 3 doses of about 3.5×10⁹ NK cells administered over about 28 days. In several embodiments, a dosing cycle comprises 3 doses of about 4×10⁹ NK cells administered over about 28 days. In several embodiments, a dosing cycle comprises 4.5 doses of about 3×10⁹ NK cells administered over about 28 days. In several embodiments, a dosing cycle comprises 3 doses of about 5×10⁹ NK cells administered over about 28 days.

In several embodiments, a dosing cycle comprises 3 doses. In some embodiments, a dosing cycle is about 42 days. In several embodiments, dosing is, for example, 3 doses of between about 3×10⁸ CAR-expressing NK cells and about 5×10⁹ CAR-expressing NK cells administered over a dosing cycle of about 42 days. In several embodiments, dosing is, for example, 3 doses of about 3×10⁸ CAR-expressing NK cells, about 1×10⁹ CAR-expressing NK cells, about 1.5×10⁹ CAR-expressing NK cells, about 2×10⁹ CAR-expressing NK cells, about 2.5×10⁹ CAR-expressing NK cells, about 3×10⁹ CAR-expressing NK cells, about 3.5×10⁹ CAR-expressing NK cells, about 4×10⁹ CAR-expressing NK cells, about 4.5×10⁹ CAR-expressing NK cells, or about 5×10⁹ CAR-expressing NK cells administered over about 42 days. In several embodiments, a dosing cycle comprises 3 doses of about 3×10⁸ NK cells administered over about 42 days. In several embodiments, a dosing cycle comprises 3 doses of about 5×10⁸ NK cells administered over about 42 days. In several embodiments, a dosing cycle comprises 3 doses of about 1×10⁹ NK cells administered over about 42 days. In several embodiments, a dosing cycle comprises 3 doses of about 1.5×10⁹ NK cells administered over about 42 days. In several embodiments, a dosing cycle comprises 3 doses of about 2×10⁹ NK cells administered over about 42 days. In several embodiments, a dosing cycle comprises 3 doses of about 2.5×10⁹ NK cells administered over about 42 days. In several embodiments, a dosing cycle comprises 3 doses of about 3×10⁹ NK cells administered over about 42 days. In several embodiments, a dosing cycle comprises 3 doses of about 3.5×10⁹ NK cells administered over about 42 days. In several embodiments, a dosing cycle comprises 3 doses of about 4×10⁹ NK cells administered over about 42 days. In several embodiments, a dosing cycle comprises 4.5 doses of about 3×10⁹ NK cells administered over about 42 days. In several embodiments, a dosing cycle comprises 3 doses of about 5×10⁹ NK cells administered over about 42 days.

In several embodiments, the first dose is administered on the first day of the dosing cycle (e.g., Day 0). In several embodiments, the second dose is administered about 7 days after administration of the first dose (e.g., Day 7). In several embodiments, the third dose is administered about 7 days after administration of the second dose (e.g., Day 14). In several embodiments, the first dose is administered on the first day of the dosing cycle (e.g., Day 0), the second dose is administered about 7 days after administration of the first dose (e.g., Day 7), and the third dose is administered about 7 days after administration of the second dose (e.g., Day 14).

In some embodiments, about 3×10⁸ NK cells (e.g., CAR-expressing NK cells) are administered on Day 0, about 3×10⁸ NK cells (e.g., CAR-expressing NK cells) are administered on Day 7, and about 3×10⁸ NK cells (e.g., CAR-expressing NK cells) are administered on Day 14. In some embodiments, about 1×10⁹ NK cells (e.g., CAR-expressing NK cells) are administered on Day 0, about 1×10⁹ NK cells (e.g., CAR-expressing NK cells) are administered on Day 7, and about 1×10⁹ NK cells (e.g., CAR-expressing NK cells) are administered on Day 14. In some embodiments, about 1.5×10⁹ NK cells (e.g., CAR-expressing NK cells) are administered on Day 0, about 1.5×10⁹ NK cells (e.g., CAR-expressing NK cells) are administered on Day 7, and about 1.5×10⁹ NK cells (e.g., CAR-expressing NK cells) are administered on Day 14.

In several embodiments, the first dose is administered on the first day of the dosing cycle (e.g., Day 0). In several embodiments, the second dose is administered between 2-4 days after administration of the first dose. In some embodiments, the second dose is administered about 2 days after administration of the first dose (e.g., Day 2). In several embodiments, the second dose is administered about 3 days after administration of the first dose (e.g., Day 3). In several embodiments, the second dose is administered about 4 days after administration of the first dose (e.g., Day 3). In some embodiments, the second dose is administered on Day 2 of the dosing cycle. In some embodiments, the second dose is administered on Day 3 of the dosing cycle. In some embodiments, the second dose is administered on Day 4 of the dosing cycle.

In several embodiments, the third dose is administered between 2-4 days after administration of the second dose (e.g., Day 7). In several embodiments, the third dose is administered about 2 days after administration of the second dose. In several embodiments, the third dose is administered about 3 days after administration of the second dose. In several embodiments, the third dose is administered about 4 days after administration of the second dose (e.g., Day 7). In some embodiments, the third dose is administered on Day 4 of the dosing cycle. In some embodiments, the third dose is administered on Day 5 of the dosing cycle. In some embodiments, the third dose is administered on Day 6 of the dosing cycle. In some embodiments, the third dose is administered on Day 7 of the dosing cycle.

In some embodiments, each dose is separated by between about 24 hours and about 72 hours. In some embodiments, each dose is separated by at least about 24 hours. In some embodiments, each dose is separated by about 24 hours. In some embodiments, each dose is separated by at least about 48 hours. In some embodiments, each dose is separated by about 48 hours. In some embodiments, each dose is separated by at least about 72 hours. In some embodiments, each dose is separated by about 72 hours.

In several embodiments, the first dose is administered on the first day of the dosing cycle (e.g., Day 0), the second dose is administered 2-4 after administration of the first dose, and the third dose is administered 2-4 days after administration of the second dose.

In several embodiments, the first dose is administered on the first day of the dosing cycle (e.g., Day 0), the second dose is administered about 2 days after administration of the first dose (e.g., Day 2), and the third dose is administered about 2 days after administration of the second dose (e.g., Day 4). In some embodiments, about 1.5×10⁹ NK cells (e.g., CAR-expressing NK cells) are administered on Day 0, about 1.5×10⁹ NK cells (e.g., CAR-expressing NK cells) are administered on Day 2, and about 1.5×10⁹ NK cells (e.g., CAR-expressing NK cells) are administered on Day 4. In some embodiments, about 2×10⁹ NK cells (e.g., CAR-expressing NK cells) are administered on Day 0, about 2×10⁹ NK cells (e.g., CAR-expressing NK cells) are administered on Day 2, and about 2×10⁹ NK cells (e.g., CAR-expressing NK cells) are administered on Day 4. In some embodiments, about 2.5×10⁹ NK cells (e.g., CAR-expressing NK cells) are administered on Day 0, about 2.5×10⁹ NK cells (e.g., CAR-expressing NK cells) are administered on Day 2, and about 2.5×10⁹ NK cells (e.g., CAR-expressing NK cells) are administered on Day 4. In some embodiments, about 3×10⁹ NK cells (e.g., CAR-expressing NK cells) are administered on Day 0, about 3×10⁹ NK cells (e.g., CAR-expressing NK cells) are administered on Day 2, and about 3×10⁹ NK cells (e.g., CAR-expressing NK cells) are administered on Day 4.

In several embodiments, the first dose is administered on the first day of the dosing cycle (e.g., Day 0), the second dose is administered about 2 days after administration of the first dose (e.g., Day 2), and the third dose is administered about 3 days after administration of the second dose (e.g., Day 5). In some embodiments, about 1.5×10⁹ NK cells (e.g., CAR-expressing NK cells) are administered on Day 0, about 1.5×10⁹ NK cells (e.g., CAR-expressing NK cells) are administered on Day 2, and about 1.5×10⁹ NK cells (e.g., CAR-expressing NK cells) are administered on Day 5. In some embodiments, about 2×10⁹ NK cells (e.g., CAR-expressing NK cells) are administered on Day 0, about 2×10⁹ NK cells (e.g., CAR-expressing NK cells) are administered on Day 2, and about 2×10⁹ NK cells (e.g., CAR-expressing NK cells) are administered on Day 5. In some embodiments, about 2.5×10⁹ NK cells (e.g., CAR-expressing NK cells) are administered on Day 0, about 2.5×10⁹ NK cells (e.g., CAR-expressing NK cells) are administered on Day 2, and about 2.5×10⁹ NK cells (e.g., CAR-expressing NK cells) are administered on Day 5. In some embodiments, about 3×10⁹ NK cells (e.g., CAR-expressing NK cells) are administered on Day 0, about 3×10⁹ NK cells (e.g., CAR-expressing NK cells) are administered on Day 2, and about 3×10⁹ NK cells (e.g., CAR-expressing NK cells) are administered on Day 5.

In several embodiments, the first dose is administered on the first day of the dosing cycle (e.g., Day 0), the second dose is administered about 3 days after administration of the first dose (e.g., Day 3), and the third dose is administered about 2 days after administration of the second dose (e.g., Day 5). In some embodiments, about 1.5×10⁹ NK cells (e.g., CAR-expressing NK cells) are administered on Day 0, about 1.5×10⁹ NK cells (e.g., CAR-expressing NK cells) are administered on Day 3, and about 1.5×10⁹ NK cells (e.g., CAR-expressing NK cells) are administered on Day 5. In some embodiments, about 2×10⁹ NK cells (e.g., CAR-expressing NK cells) are administered on Day 0, about 2×10⁹ NK cells (e.g., CAR-expressing NK cells) are administered on Day 3, and about 2×10⁹ NK cells (e.g., CAR-expressing NK cells) are administered on Day 5. In some embodiments, about 2.5×10⁹ NK cells (e.g., CAR-expressing NK cells) are administered on Day 0, about 2.5×10⁹ NK cells (e.g., CAR-expressing NK cells) are administered on Day 3, and about 2.5×10⁹ NK cells (e.g., CAR-expressing NK cells) are administered on Day 5. In some embodiments, about 3×10⁹ NK cells (e.g., CAR-expressing NK cells) are administered on Day 0, about 3×10⁹ NK cells (e.g., CAR-expressing NK cells) are administered on Day 3, and about 3×10⁹ NK cells (e.g., CAR-expressing NK cells) are administered on Day 5.

In several embodiments, the first dose is administered on the first day of the dosing cycle (e.g., Day 0), the second dose is administered about 3 days after administration of the first dose (e.g., Day 3), and the third dose is administered about 3 days after administration of the second dose (e.g., Day 6). In some embodiments, about 1.5×10⁹ NK cells (e.g., CAR-expressing NK cells) are administered on Day 0, about 1.5×10⁹ NK cells (e.g., CAR-expressing NK cells) are administered on Day 3, and about 1.5×10⁹ NK cells (e.g., CAR-expressing NK cells) are administered on Day 6. In some embodiments, about 2×10⁹ NK cells (e.g., CAR-expressing NK cells) are administered on Day 0, about 2×10⁹ NK cells (e.g., CAR-expressing NK cells) are administered on Day 3, and about 2×10⁹ NK cells (e.g., CAR-expressing NK cells) are administered on Day 6. In some embodiments, about 2.5×10⁹ NK cells (e.g., CAR-expressing NK cells) are administered on Day 0, about 2.5×10⁹ NK cells (e.g., CAR-expressing NK cells) are administered on Day 3, and about 2.5×10⁹ NK cells (e.g., CAR-expressing NK cells) are administered on Day 6. In some embodiments, about 3×10⁹ NK cells (e.g., CAR-expressing NK cells) are administered on Day 0, about 3×10⁹ NK cells (e.g., CAR-expressing NK cells) are administered on Day 3, and about 3×10⁹ NK cells (e.g., CAR-expressing NK cells) are administered on Day 6.

In several embodiments, the first dose is administered on the first day of the dosing cycle (e.g., Day 0), the second dose is administered about 3 days after administration of the first dose (e.g., Day 3), and the third dose is administered about 4 days after administration of the second dose (e.g., Day 7). In some embodiments, about 1×10⁹ NK cells (e.g., CAR-expressing NK cells) are administered on Day 0, about 1×10⁹ NK cells (e.g., CAR-expressing NK cells) are administered on Day 3, and about 1×10⁹ NK cells (e.g., CAR-expressing NK cells) are administered on Day 7. In some embodiments, about 1.5×10⁹ NK cells (e.g., CAR-expressing NK cells) are administered on Day 0, about 1.5×10⁹ NK cells (e.g., CAR-expressing NK cells) are administered on Day 3, and about 1.5×10⁹ NK cells (e.g., CAR-expressing NK cells) are administered on Day 7. In some embodiments, about 2×10⁹ NK cells (e.g., CAR-expressing NK cells) are administered on Day 0, about 2×10⁹ NK cells (e.g., CAR-expressing NK cells) are administered on Day 3, and about 2×10⁹ NK cells (e.g., CAR-expressing NK cells) are administered on Day 7. In some embodiments, about 2.5×10⁹ NK cells (e.g., CAR-expressing NK cells) are administered on Day 0, about 2.5×10⁹ NK cells (e.g., CAR-expressing NK cells) are administered on Day 3, and about 2.5×10⁹ NK cells (e.g., CAR-expressing NK cells) are administered on Day 7. In some embodiments, about 3×10⁹ NK cells (e.g., CAR-expressing NK cells) are administered on Day 0, about 3×10⁹ NK cells (e.g., CAR-expressing NK cells) are administered on Day 3, and about 3×10⁹ NK cells (e.g., CAR-expressing NK cells) are administered on Day 7.

In particular, methods of treating subjects with dosing regimens as provided herein and described in the Working Examples (e.g., with NK cells engineered to express a CD19 CAR) may result in unexpected efficacy and safety, including an efficacy and safety profile allowing for outpatient administration. It is contemplated that such unexpected effects are due, at least in part, to providing an increased number of genetically engineered NK cells during a time period in which a subject's immune response has not yet fully recovered following lymphodepleting therapy, thereby allowing increased engraftment of adoptively transferred immune cells (e.g., NK cells). Such a time period may include, for example, within about 7 days of administration of the genetically engineered NK cells and/or within about 14 days of administration of a first dose of lymphodepleting therapy. For example, by providing higher doses of genetically engineered NK cells, an increased number of doses, or both, within this time period, the efficacy of genetically engineered NK cells may be improved. Further, the opportunity to provide higher and/or an increased number of doses of genetically engineered NK cells is afforded, inter alia, by the lack of graft vs. host disease and toxicity associated with NK cells. This is in contrast with, for example, cytokine release syndrome (CRS) and neurotoxicity frequently observed with adoptive transfer of engineered T cells, such as CAR T cells.

In several embodiments, a lymphodepletion process is performed prior to the first dose. In several embodiments, the administration of engineered NK cells is preceded by one or more preparatory treatments. In several embodiments, the administration of engineered NK cells is preceded by a lymphodepleting therapy (also referred to as “lymphodepletion”). Thus, also provided herein is a method of preparing a subject having an autoimmune disease for treatment with a composition comprising natural killer (NK) cells genetically engineered to express a chimeric antigen receptor (CAR) that binds to CD19, the method comprising administering a lymphodepleting therapy to the subject prior to administration of the composition to the subject. In some embodiments, the lymphodepleting therapy comprises administration of cyclophosphamide and does not comprise administration of fludarabine. Thus, in some aspects, provided herein is a method of preparing a subject having an autoimmune disease for treatment with a composition comprising natural killer (NK) cells genetically engineered to express a chimeric antigen receptor (CAR) that binds to CD19, the method comprising administering a lymphodepleting therapy consisting of cyclophosphamide to the subject prior to administration of the composition to the subject.

In several embodiments, a combination of chemotherapeutic agents is used for lymphodepletion. In several embodiments, a single chemotherapeutic agent is used for lymphodepletion. In several embodiments, wherein a combination of chemotherapeutic agents is used, agents with different mechanisms of actions are optionally used. In several embodiments, different classes of agents are optionally used. In several embodiments, an antimetabolic agent is used. In several embodiments, the antimetabolic agent inhibits and/or prevents cell replication. In several embodiments, the antimetabolic agent is an altered nucleotide that disrupts DNA replication, making it effective in targeting rapidly dividing tumor cells.

In several embodiments, cyclophosphamide is used. In several embodiments, a dose of between about 100 mg/m² and about 1000 mg/m² cyclophosphamide is administered, including doses of about 100.0 mg/m², about 200 mg/m², about 300 mg/m², about 400 mg/m², about 500 mg/m², about 600 mg/m², about 700 mg/m², about 800 mg/m², about 900 mg/m², about 1000 mg/m², or any dose between those listed. In several embodiments, a dose of about 300 mg/m² of cyclophosphamide is administered. In several embodiments, a dose of about 500 mg/m² of cyclophosphamide is administered. In several embodiments, the dose of cyclophosphamide is given daily for at least about 2 days, about 3 days, about 4 days, about 5 days, about 6 days, or about 7 days. In several embodiments, the dose of cyclophosphamide is given daily for about 3 days. In several embodiments, a dose of about 500 mg/m² of cyclophosphamide is administered on each of 5 days, 4 days, and 3 days prior to administration of the composition comprising NK cells genetically engineered to express a CAR. In several embodiments, a dose of about 300 mg/m² of cyclophosphamide is administered on each of Days −3, −4, and −5. In several embodiments, a dose of about 500 mg/m² of cyclophosphamide is administered on each of Days −3, −4, and −5. In several embodiments, if necessary, the dose can be split and given, for example, twice daily.

In some embodiments, the lymphodepleting therapy comprises administration of cyclophosphamide. In some embodiments, the lymphodepleting therapy comprises administration of a single dose of cyclophosphamide. In some embodiments, the lymphodepleting therapy consists of administration of a single dose of cyclophosphamide. Thus, in some aspects, provided herein is a method of preparing a subject having an autoimmune disease for treatment with a composition comprising natural killer (NK) cells genetically engineered to express a chimeric antigen receptor (CAR) that binds to CD19, the method comprising administering a lymphodepleting therapy consisting of a single dose of cyclophosphamide to the subject prior to administration of the composition to the subject.

In some embodiments, a single dose of cyclophosphamide comprises between about 500 mg/m² and about 1500 mg/m² cyclophosphamide. In some embodiments, a single dose of cyclophosphamide is about 500 mg/m² cyclophosphamide. In some embodiments, a single dose of cyclophosphamide is about 600 mg/m² cyclophosphamide. In some embodiments, a single dose of cyclophosphamide is about 700 mg/m² cyclophosphamide. In some embodiments, a single dose of cyclophosphamide is about 750 mg/m² cyclophosphamide. In some embodiments, a single dose of cyclophosphamide is about 800 mg/m² cyclophosphamide. In some embodiments, a single dose of cyclophosphamide is about 900 mg/m² cyclophosphamide. In some embodiments, a single dose of cyclophosphamide comprises about 1000 mg/m² cyclophosphamide. In some embodiments, a single dose of cyclophosphamide is about 1000 mg/m² cyclophosphamide. In some embodiments, a single dose of cyclophosphamide is about 1100 mg/m² cyclophosphamide. In some embodiments, a single dose of cyclophosphamide is about 1200 mg/m² cyclophosphamide. In some embodiments, a single dose of cyclophosphamide is about 1250 mg/m² cyclophosphamide. In some embodiments, a single dose of cyclophosphamide is about 1300 mg/m² cyclophosphamide. In some embodiments, a single dose of cyclophosphamide is about 1400 mg/m² cyclophosphamide. In some embodiments, a single dose of cyclophosphamide is about 1500 mg/m² cyclophosphamide. In some embodiments, a single dose of cyclophosphamide is administered about 3 days prior to administration of the composition comprising NK cells genetically engineered to express a CAR. In some embodiments, a single dose of about 1000 mg/m² cyclophosphamide is administered about 3 days prior to administration of the composition comprising NK cells genetically engineered to express a CAR. In some embodiments, a single dose of about 1000 mg/m² cyclophosphamide is administered on Day −3.

In several embodiments, an additional agent is used in combination with cyclophosphamide. In several embodiments, the additional agent is also an antimetabolite. In several embodiments, the additional agent inhibits one or more of DNA polymerase alpha, ribonucleotide reductase and/or DNA primase, thus inhibiting DNA synthesis.

In several embodiments, the additional agent is fludarabine. In several embodiments, a dose of between about 5.0 mg/m² and about 200 mg/m² fludarabine is administered, including doses of about 5.0 mg/m², about 10.0 mg/m², about 15.0 mg/m², about 20.0 mg/m², about 25.0 mg/m², about 30.0 mg/m², about 35.0 mg/m², about 40.0 mg/m², about 45.0 mg/m², about 50.0 mg/m², about 60.0 mg/m², about 70.0 mg/m², about 80.0 mg/m², about 90.0 mg/m², about 100.0 mg/m², about 125.0 mg/m², about 150.0 mg/m², about 175.0 mg/m², about 200.0 mg/m², or any dose between those listed. In several embodiments, a dose of about 30 mg/m² of fludarabine is administered. In several embodiments, the dose of fludarabine is given daily for at least about 2 days, about 3 days, about 4 days, about 5 days, about 6 days, or about 7 days. In several embodiments, the dose of fludarabine is given daily for about 3 days. In several embodiments, the dose of fludarabine is given daily for about 5 days. In several embodiments, if necessary, the dose can be split and given, for example, twice daily.

In some embodiments, the lymphodepleting therapy comprises administration of fludarabine. In some embodiments, the lymphodepleting therapy comprises administration of fludarabine at between about 20 mg/m² and about 40 mg/m² daily. In some embodiments, the lymphodepleting therapy comprises administration of fludarabine at about 20 mg/m² daily. In some embodiments, the lymphodepleting therapy comprises administration of fludarabine at about 25 mg/m² daily. In some embodiments, the lymphodepleting therapy comprises administration of fludarabine at about 30 mg/m² daily. In some embodiments, the lymphodepleting therapy comprises administration of fludarabine at about 40 mg/m² daily. In some embodiments, the lymphodepleting therapy comprises administration of fludarabine daily for 2-4 days. In some embodiments, the lymphodepleting therapy comprises administration of fludarabine daily for 2 days. In some embodiments, the lymphodepleting therapy comprises administration of fludarabine daily for 3 days. In some embodiments, the lymphodepleting therapy comprises administration of fludarabine daily for 4 days. In some embodiments, the lymphodepleting therapy comprises administration of 25 mg/m² fludarabine daily for 3 days. In some embodiments, the lymphodepleting therapy comprises administration of 25 mg/m² fludarabine daily on each of 5, 4, and 3 days prior to administration of the composition comprising NK cells genetically engineered to express a CAR. In some embodiments, the lymphodepleting therapy comprises administration of 25 mg/m² fludarabine daily on each of Days −5, −4, and −3. In some embodiments, the lymphodepleting therapy comprises administration of 30 mg/m² fludarabine daily for 3 days. In some embodiments, the lymphodepleting therapy comprises administration of 30 mg/m² fludarabine daily on each of 5, 4, and 3 days prior to administration of the composition comprising NK cells genetically engineered to express a CAR. In some embodiments, the lymphodepleting therapy comprises administration of 30 mg/m² fludarabine daily on each of Days −5, −4, and −3.

In several embodiments, a combination of fludarabine and cyclophosphamide is used with a daily dose of fludarabine of between about 20 mg/m² and 40 mg/m² and a daily dose of cyclophosphamide of between about 200 mg/m² and 600 mg/m². In several embodiments, cyclophosphamide (300 mg/m²) and fludarabine (30 mg/m²) are administered daily for 3 days. In several embodiments, cyclophosphamide (500 mg/m²) and fludarabine (30 mg/m²) are administered daily for 3 days. In some embodiments, fludarabine and cyclophosphamide are each administered daily 5 days, 4 days, and 3 days prior to administration of the engineered NK cells.

In some embodiments, the lymphodepleting therapy comprises administration of a single dose of cyclophosphamide and three daily doses of fludarabine.

In some embodiments, the lymphodepleting therapy comprises administration of a single dose of about 1000 mg/m² cyclophosphamide and three daily doses of about 25 mg/m² fludarabine. In some embodiments, the lymphodepleting therapy comprises administration of a single dose of about 1000 mg/m² cyclophosphamide about 3 days prior to administration of the composition comprises NK cells genetically engineered to express a CAR and administration of a dose of about 25 mg/m² fludarabine on each of 5, 4, and 3 days prior to administration of the composition comprising NK cells genetically engineered to express a CAR. In some embodiments, the lymphodepleting therapy comprises administration of a single dose of about 1000 mg/m² cyclophosphamide on Day-3 and a dose of about 25 mg/m² fludarabine on each of Days −5, −4, and −3.

In some embodiments, the lymphodepleting therapy comprises administration of a single dose of about 1000 mg/m² cyclophosphamide and three daily doses of about 30 mg/m² fludarabine. In some embodiments, the lymphodepleting therapy comprises administration of a single dose of about 1000 mg/m² cyclophosphamide about 3 days prior to administration of the composition comprises NK cells genetically engineered to express a CAR and administration of a dose of about 30 mg/m² fludarabine on each of 5, 4, and 3 days prior to administration of the composition comprising NK cells genetically engineered to express a CAR. In some embodiments, the lymphodepleting therapy comprises administration of a single dose of about 1000 mg/m² cyclophosphamide on Day −3 and a dose of about 30 mg/m² fludarabine on each of Days −5, −4, and −3.

In some embodiments, the lymphodepleting therapy comprises administration of three daily doses of about 500 mg/m² cyclophosphamide and three daily doses of about 30 mg/m² fludarabine. In some embodiments, the lymphodepleting therapy comprises administration of a dose of about 30 mg/m² fludarabine and a dose of about 500 mg/m² cyclophosphamide on each of 5, 4, and 3 days prior to administration of the composition comprising NK cells genetically engineered to express a CAR. In some embodiments, the lymphodepleting therapy comprises administration of a dose of about 500 mg/m² cyclophosphamide on each of Days −3, −4, and −5, and a dose of about 30 mg/m² fludarabine on each of Days −5, −4, and −3.

In several embodiments, the lymphodepletion regimen works synergistically with the engineered NK cells to provide effect reduction and/or elimination of cells that cause or mediate the disease.

In certain embodiments, a dose of a genetically engineered cell(s) described herein or composition thereof is administered to a subject every day, every other day, every couple of days, every third day, once a week, twice a week, three times a week, or once every two weeks. In other embodiments, two, three or four doses of a genetically engineered cell(s) described herein or composition thereof is administered to a subject every day, every couple of days, every third day, once a week or once every two weeks. In some embodiments, a dose(s) of a genetically engineered cell(s) described herein or composition thereof is administered for 2 days, 3 days, 5 days, 7 days, 14 days, or 21 days. In certain embodiments, a dose of a genetically engineered cell(s) described herein or composition thereof is administered for 1 month, 1.5 months, 2 months, 2.5 months, 3 months, 4 months, 5 months, 6 months or more.

In several embodiments, a subject is subject to lymphodepletion at least one time prior to administration of genetically engineered cells as disclosed herein. In several embodiments, lymphodepletion is performed before one or more additional doses of engineered cells are administered. In several embodiments, a dosing cycle is used that comprises lymphodepletion followed by at least two doses of engineered cells as disclosed herein, with the two doses separated by a time interval. In several embodiments, the time interval is 2, 3, 4, 5, 6, 7, 8, 9, 10, 11, 12, 13, 14, 15, 16, 17, 18, 19, 20, 21, or more days (including intervals falling between the time marking a price interval since the last administration, e.g., 84 hours, or 3.5 days). In several embodiments, the dosing cycle itself is approximately 14, 21, 28, 35, 42 or more days. In several embodiments, three doses are administered, ˜1 week apart from each other. In several embodiments, two doses are administered ˜1 week apart from one another. In several embodiments, a subject receives a first dose on day 0 of the cycle, a second dose on day 7 of the cycle and a third dose on day 14 of the cycle. In several such embodiments, a 28-day cycle is used with primary outcome measures evaluated at day 28 (see e.g., FIG. 2). In several embodiments, a subject receives a first dose on day 0 of the cycle and a second dose on day 7 of the cycle. In several such embodiments, a 28-day cycle is used with primary outcome measures evaluated at day 28 (see e.g., FIG. 2A). In several embodiments, a subject receives a first dose on day 0 of the cycle, a second dose on day 7 of the cycle and a third dose on day 14 of the cycle. In several embodiments, a subject receives a first dose on day 0 of the cycle, a second dose on day 3 of the cycle and a third dose on day 7 of the cycle. In several such embodiments, a 42-day cycle is used with primary outcome measures evaluated at day 41 (see e.g., FIGS. 2B-D). In several such embodiments, a 42-day cycle is used with primary outcome measures evaluated at day 41 (see e.g., FIGS. 2B-D).

In several embodiments, lymphodepletion is performed prior to the inception of each dosing cycle, if subsequent dosing cycles are required (e.g., the subject requires further treatment). For example, in several embodiments, a subject undergoes lymphodepletion, receives a plurality of doses of engineered cells according to a cycle, is evaluated at the end of the cycle time and, if deemed necessary undergoes a second lymphodepletion followed by a second dosing cycle. In such embodiments where multiple dosing cycles are used, a lymphodepleting therapy is only administered prior to the first dosing cycle. In such embodiments where multiple dosing cycles are used, a first and a second dosing cycle need not be the same (e.g., a first cycle may have 2 doses, while a second uses three doses). Depending on the subject 1, 2, 3, 4, 5, 6, 7, 8, 9, 10, or more dosing cycles are performed.

In some embodiments, the administration effectively treats the subject despite the subject having become resistant to a prior line of therapy. In some embodiments, at least 30%, at least 35%, at least 40% or at least 50% of subjects treated according to the method achieve a clinical response; and/or at least about 40%, at least about 50%, at least about 60% or at least about 70% of the subjects treated according to the method achieve a clinical response. In some embodiments, at least 30%, at least 35%, at least 40% or at least 50% of subjects treated according to the method achieve a clinical response. In some embodiments, at least about 40%, at least about 50%, at least about 60% or at least about 70% of the subjects treated according to the method achieve a clinical response. In some embodiments, at least 30% of subjects treated according to the method achieve a clinical response. In some embodiments, at least 40% of subjects treated according to the method achieve a clinical response. In some embodiments, at least 50% of subjects treated according to the method achieve a clinical response. In some embodiments, at least 60% of subjects treated according to the method achieve a clinical response. In some embodiments, at least 70% of subjects treated according to the method achieve a clinical response. In some embodiments, at least 80% of subjects treated according to the method achieve a clinical response.

In some embodiment, a clinical response comprises a reduction in disease activity (e.g., as determined by a disease activity index, including any of those known in the art).

In some embodiments, a subject has SLE, and the disease activity index is SLE Responder Index-4 (SRI-4). In some aspects, a response on SRI-4 is defined as achieving each of: ≥4-point reduction in the SELENA-SLEDAI score, no new British Isles Lupus Assessment Group (BILAG) A organ domain score or 2 new BILAG B organ domain scores, and no worsening (<0.30-point increase) in Physicians Global Assessment (PGA) score. In some embodiments, a subject has SLE, and the disease activity index is PGA. In some embodiments, a response on PGA is defined as a decrease in score. In some embodiments, a subject has SLE, and the disease activity index is SELENA-SLEDAI. In some embodiments, a response on SELENDA-SLEDAI is defined as a decrease in score. In some embodiments, a subject has LN, and the disease activity index is Primary Efficacy Renal Response (PERR). In some embodiments, a response on PERR is defined as achieving: urine protein: creatinine ratio (uPCR) ≤0.7 g/g and estimated glomerular filtration rate (cGFR) ≥60 mL/min/1.73 m2 or no decrease in eGFR of >20% from pre-flare value. In some embodiments, a subject has LN, and the disease activity index is SELENA-SLEDAI. In some embodiments, a response on SELENDA-SLEDIA is defined as a decrease in score.

In some embodiments, a subject has IIM (also known as myositis), and the disease activity index is selected from the group consisting of 2017 ULAR/ACR, Disease Activity Score (DAS), Myositis Damage Index (MDI), Short-Form 36 (SF-36), CHQ-PF50, Childhood Myositis Assessment Scale (CMAS), Manual Muscle Testing (MMT), Quantitative Muscle Testing (QMT), IBM Functional Rating Score (IBMFRS), Myositis Disease Activity Assessment Tool (MDAAT), Myositis Functional Index-2 or -3 (FI-2 or FI-3), Total Improvement Score (TIS), Cutaneous Dermatomyositis Disease Area and Severity Index Total Activity Score (CDASI-A), and Dermatomyositis Skin Severity Index (DSSI). In some embodiments, a subject has IIM, and the disease activity index is 2017 ULAR/ACR. In some embodiments, a subject has IIM, and the disease activity index is DAS. In some embodiments, a subject has IIM, and the disease activity index is MDI. In some embodiments, a subject has IIM, and the disease activity index is SF-36. In some embodiments, a subject has IIM, and the disease activity index is CHQ-PF50. In some embodiments, a subject has IIM, and the disease activity index is CMAX. In some embodiments, a subject has IIM, and the disease activity index is MMT. In some embodiments, a subject has IIM, and the disease activity index is QMT. In some embodiments, a subject has IIM, and the disease activity index is IBMFRS. In some embodiments, a subject has IIM, and the disease activity index is MDAAT. In some embodiments, a subject has IIM, and the disease activity index is FI-2 or FI-3. In some embodiments, a subject has IIM, and the disease activity index is TIS. In some embodiments, a subject has IIM, and the disease activity index is CDASI-A. In some embodiments, a subject has IIM, and the disease activity index is DSSI. In some embodiments, the subject has myositis and the disease activity index is Health Assessment Questionnaire Disability Index (HAQ-DI). In some embodiments, the subject has myositis and the disease activity index is PhGA. In some embodiments, the subject has myositis and the disease activity index is PGA.

In some embodiments, a subject has MG, and the disease activity index is selected from the group consisting of Quantitative Myasthenia Gravis (QMG), Myasthenia Gravis Activities of Daily Living (MG-ADL), Myasthenia Gravis Composite (MGC), and MGFA Post-Intervention Status (MGFA-PIS). In some embodiments, a subject has MG, and the disease activity index is QMG. In some embodiments, a subject has MG, and the disease activity index is MG-ADL. In some embodiments, a subject has MG, and the disease activity index is MGC. In some embodiments, a subject has MG, and the disease activity index is MGFA-PIS. In some embodiments, a subject has MG, and the disease activity index is MG Quality of Life 15-items (QOL15).

In some embodiments, the subject has scleroderma, and the disease activity index is selected from the group consisting of 2013 ACR-EULAR Classification of SSc, the Localized Scleroderma Assessment Tool (LoSCAT), Modified Rodnan Skin Score (MRSS). In some embodiments, the subject has scleroderma, and the disease activity index is 2013 ACR-EULAR Classification of SSc. In some embodiments, the subject has scleroderma, and the disease activity index is LoSCAT. In some embodiments, the subject has scleroderma, and the disease activity index is MRSS. In some embodiments, the subject has scleroderma, and the disease activity index is Forced Vital Capacity (FVC). In some embodiments, the subject has scleroderma, and the disease activity index is Diffuse Capacity of the Lungs for Carbon Monoxide (DLCO). In some embodiments, the subject has scleroderma, and the disease activity index is European Sclerosis Trial and Research Activity Index (EUSTAR). In some embodiments, the subject has scleroderma and the disease activity index is The European Scleroderma Clinical Trials Consortium Scleroderma Skin Severity Score-Analog Instrument (EScSG AI). In some embodiments, the subject has scleroderma, and the disease activity index is 6-Minute Walk Distance (6MWD). In some embodiments, the subject has scleroderma, and the disease activity index is Systemic Sclerosis-Associated Interstitial Lung Disease (SSc-ILD). In some embodiments, the subject has scleroderma, and the disease activity index is Physician Global Assessment (PhGA). In some embodiments, the subject has scleroderma, and the disease activity index is Patient Global Assessment (PGA). In some embodiments, the subject has scleroderma and the disease activity index is Health Assessment Questionnaire Disability Index (HAQ-DI). In some embodiments, the subject has scleroderma, and the disease activity index is Combined Response Index in Systemic Sclerosis (CRISS).

In some embodiments, the subject has vasculitis (e.g., ANCA vasculitis), and the disease activity index is selected from the group consisting of Vasculitis Damage Index (VDI), Birmingham Vasculitis Activity Score (BVAS), and the Five Factor Score (FFS). In some embodiments, the subject has vasculitis, and the disease activity index is VDI. In some embodiments, the subject has vasculitis, and the disease activity index is BVAS. In some embodiments, the subject has vasculitis, and the disease activity index is FFS.

In some embodiments, a subject has RA, and the disease activity index is selected from the group consisting of American College of Rheumatology 20% improvement criteria (ACR20), ACR50, and ACR70 response rates, hybrid ACR response, and the 28-joint disease activity score (DAS28). In some embodiments, a subject has RA, and the disease activity index is ACR20. In some embodiments, a subject has RA, and the disease activity index is ACR50. In some embodiments, a subject has RA, and the disease activity index is ACR70. In some embodiments, a subject has RA, and the disease activity index is hybrid ACR response. In some embodiments, a subject has RA, and the disease activity index is DAS28.

In some embodiments, a subject has Sjogren's syndrome and the disease activity index is selected from the group consisting of ESSDAI, ClinESSDAI, ESSPRI, Schirmer/OSS, UWS/SGUS, RF/IgG, or any combination thereof. In some embodiments, a subject has Sjogren's syndrome and the disease activity index is ESSDAI. In some embodiments, a subject has Sjogren's syndrome and the disease activity index is ClinESSDAI. In some embodiments, a subject has Sjogren's syndrome and the disease activity index is ESSPRI.

In some embodiments, a subject has MS, and the disease activity index is the Multiple Sclerosis Disease Activity (MSDA) Test (Octave). In some embodiments, a subject has MS, and the disease activity index is the Expanded Disability Status Scale (EDSS).

In some embodiments, a clinical response comprises a reduction in flare or an increase in time to flare. In some embodiments, a clinical response comprises a reduction in flare. In some embodiments, a clinical response comprises an increase in time to flare. In some aspects, a method or use as provided herein can increase the average time between a subject's flares, thereby decreasing a need for induction therapy and/or a long-term maintenance immunosuppressant regimen.

In some embodiments, the flare is a SLE flare. In some embodiments, the flare is a renal flare. Definitions of flares (including renal flares) are known and described in the art. As a non-limiting example, EULAR defines a SLE flare or relapse as an increase in disease activity requiring more-intensive treatment, and a renal flare as an increase in proteinuria or serum creatinine level, an abnormal urinary sediment or a reduction in creatinine clearance due to active disease (Gordon et al., Lupus (2009) 18:257-63).

In some embodiments, the flare is a MS flare (also known as a relapse, flare-up, or attack). In some embodiments, a MS flare comprises the occurrence of new symptoms or the worsening of old symptoms. In some embodiments, a MS flare comprises the occurrence of new symptoms. In some embodiments, a MS flare comprises the worsening of old symptoms

In some embodiments, a clinical response comprises a partial renal response (PRR). In some embodiments, a clinical response comprises a complete renal response (CRR). In some embodiments, the PRR or CRR is determined by EULAR or ERA-EDTA criteria. In some embodiments, the PRR or CRR is determined by EULAR criteria. In some embodiments, the PRR or CRR is determined by ERA-EDTA criteria. In some aspects, a complete renal response is defined as uPCR <0.5 g/g and eGFR ≥90 mL/min/1.73 m² or no decrease in eGFR of >10% from pre-flare value.

In some embodiments, a clinical response comprises complete remission. In some embodiments, a clinical response comprises remission maintenance.

In some embodiments, at least 30%, at least 35%, at least 40% or at least 50% of subjects treated according to the method achieve a complete clinical response or remission (e.g., complete absence of disease activity as determined by a disease activity index, including any of those known in the art). In some embodiments, at least about 40%, at least about 50%, at least about 60% or at least about 70% of the subjects treated according to the method achieve a complete clinical response or remission (e.g., complete absence of disease activity as determined by a disease activity index, including any of those known in the art).

In some embodiments, the method decreases the presence, level, and/or activity of an autoantibody. In some embodiments, at least 30%, at least 35%, at least 40% or at least 50% of subjects treated according to the method achieve a reduction in an autoantibody associated with the autoimmune disease. In some embodiments, at least about 40%, at least about 50%, at least about 60% or at least about 70% of the subjects treated according to the method achieve a reduction in an autoantibody associated with the autoimmune disease. In some embodiments, the autoantibody is an anti-AChR antibody. In some embodiments, the autoantibody is an anti-MuSK antibody. In some embodiments, the autoantibody is an anti-LRP4 antibody. In some embodiments, the autoantibody is an anti-SAE antibody. In some embodiments, the autoantibody is an anti-Mi2 antibody. In some embodiments, the autoantibody is an anti-MDA5 antibody. In some embodiments, the autoantibody is an anti-NXP2 antibody. In some embodiments, the autoantibody is an anti-TIF1 antibody. In some embodiments, the autoantibody is an anti-SRP antibody. In some embodiments, the autoantibody is an anti-HMGCR antibody. In some embodiments, the autoantibody is an anti-PM-Scl antibody. In some embodiments, the autoantibody is an anti-Scl-70 antibody. In some embodiments, the autoantibody is an anti-Ku antibody. In some embodiments, the autoantibody is an anti-RNP antibody. In some embodiments, the autoantibody is an anti-Ro/SSA antibody. In some embodiments, the autoantibody is an anti-La/SSB antibody. In some embodiments, the autoantibody is an anti-CN-1A antibody. In some embodiments, the autoantibody is an anti-centromere antibody. In some embodiments, the autoantibody is an anti-topoisomerase1 antibody. In some embodiments, the autoantibody is an anti-RNA polymerase III antibody. In some embodiments, the autoantibody is an anti-neutrophilic cytoplasmic antibody.

In some embodiments, at least 30%, at least 35%, at least 40% or at least 50% of subjects treated according to the method achieve a reduction in antiphospholipid antibodies, antinuclear antibodies (ANA), anti-double-stranded DNA antibodies (anti-dsDNA), or anti-Smith (anti-Sm) antibodies. In some embodiments, at least about 40%, at least about 50%, at least about 60% or at least about 70% of the subjects treated according to the method achieve a reduction in antiphospholipid antibodies, antinuclear antibodies (ANA), anti-double-stranded DNA antibodies (anti-dsDNA), or anti-Smith (anti-Sm) antibodies.

In some embodiments, at least 30%, at least 35%, at least 40% or at least 50% of subjects treated according to the method achieve a reduction in level of a complement protein. In some embodiments, the complement protein is one or more of C1, C2, C3, C4, C5, C6, C7, C8, and C9. In some embodiments, the complement protein is C3 or C4. In some embodiments, the complement protein is C3. In some embodiments, the complement protein is C4. In some embodiments, the complement protein is C3 and C4. In some embodiments, at least 30%, at least 35%, at least 40% or at least 50% of subjects treated according to the method achieve a reduction in C3 or C4 level. In some embodiments, at least 30%, at least 35%, at least 40% or at least 50% of subjects treated according to the method achieve a reduction in C3 level. In some embodiments, at least 30%, at least 35%, at least 40% or at least 50% of subjects treated according to the method achieve a reduction in C4 level. In some embodiments, at least 30%, at least 35%, at least 40% or at least 50% of subjects treated according to the method achieve a reduction in each of C3 and C4 level.

In some embodiments, the presence, level, or activity of a complement protein is determined by a complement blood test such as CH50. A CH50 test assesses the presence of level of each of C1, C2, C3, C4, C5, C6, C7, C8, and C9. Thus, in some embodiments, the presence or level of each of C1, C2, C3, C4, C5, C6, C7, C8, and C9 is determined by a CH50 test. Thus, in some embodiments, the level or activity of a complement protein is determined by a CH50 test. In some embodiments, at least 30%, at least 35%, at least 40% or at least 50% of subjects treated according to the method achieve a reduction in CH50.

Responses can be determined based upon any available diagnostic criteria, including the European Consensus Lupus Activity Measure (ECLAM), SLE Responder Index (SRI, e.g., SRI-4, SRI-5, SRI-6), Systemic Lupus Erythematosus Activity Measure (SLAM, e.g. SLAM-R), Systemic Lupus Erythematosus Disease Activity Index (SLEDAI, e.g. SLEDAI 2K), British Isles Lupus Assessment Group (BILAG), and the British Isles Lupus Assessment Group (BILAG)-based Composite Lupus Assessment (BICLA). In some embodiments, the response is based on ECLAM. In some embodiments, the response is based on SRI (e.g., SRI-4). In some embodiments, the response is based on SLAM. In some embodiments, the response is based on SLEDAI (e.g., SLEDAI 2K). In some embodiments, the response is based on BILAG. In some embodiments, the response is based on BICLA.

Responses such as complete renal response (CRR) and partial renal response (PRR) may also be determined based upon European Alliance of Associations for Rheumatology (EULAR) criteria and/or European Renal Association-European Dialysis and Transplant Association (ERA-EDTA) criteria. Outcomes or responses may also be assessed by Safety of Estrogens in Lupus Erythematosus National Assessment-SLE Disease Activity Index (SELENA-SLEDAI) criteria.

In some embodiments, the methods decrease the number of CD19-expressing cells in a tissue of the subject. In some embodiments, the method decreases the number of B cells in a tissue of the subject. In some embodiments, the methods decrease the number of peripheral CD19-expressing cells in the subject. In some embodiments, the method decreases the number of peripheral B cells in the subject.

In some embodiments, at least 40% or at least 50% of subjects treated according to the methods provided herein achieve a response lasting more than at or about 3 months, 6 months, 12 months, 14 months, or 16 months. In some embodiments, at least 40% or at least 50% of subjects treated according to the methods provided herein achieve remission lasting more than at or about 3 months, 6 months, 12 months, 14 months, or 16 months. In some embodiments, the measure of duration of response (DOR) includes the time from documentation of response or remission to re-emergence of symptoms. In some embodiments, at least 30%, at least 35%, at least 40% or at least 50% of subjects treated according to the method do not have detectable autoantibodies immediately after treatment; and/or at least about 40%, at least about 50%, at least about 60% or at least about 70% of the subjects treated according to the method do not have detectable autoantibodies immediately after treatment.

In several embodiments, an additional therapeutic agent is administered at least once during the lymphodepletion and/or the dosing cycle. In several embodiments, an additional therapeutic agent is administered at least once during the lymphodepletion. In several embodiments, an additional therapeutic agent is administered at least once during the dosing cycle. In some embodiments, the cells are administered as part of a combination treatment, such as simultaneously with or sequentially with, in any order, another therapeutic intervention, such as another antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents.

### IV. Combination Agents

In some embodiments, the genetically engineered cells are administered as part of a combination treatment, such as simultaneously with or sequentially with, in any order, another therapeutic intervention, such as an antibody or engineered cell or receptor or agent, such as a cytotoxic or therapeutic agent. The genetically engineered cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, a lymphodepleting therapy is administered prior to the one or more additional therapeutic agents. In some embodiments, the lymphodepleting therapy is administered after the one or more additional therapeutic agents.

Additional therapeutic agents for use in combination with the genetically engineered cells include steroids, immunnosuppresive agents, antimalarial agents, alkylating agents, B cell-targeting agents, and any combination thereof.

In some embodiments, the additional therapeutic agent is a steroid. In some embodiments, the steroid is a corticosteroid, such as a glucocorticoid. In some embodiments, the steroid is a corticosteroid. In some embodiments, the steroid is a glucocorticoid. In some embodiments, the steroid is selected from the group consisting of prednisone, methylprednisone, dexamethasone, and betamethasone. In some embodiments, the steroid is prednisone. In some embodiments, the steroid is methylprednisone. In some embodiments, the steroid is dexamethasone. In some embodiments, the steroid is betamethasone. In some embodiments, the steroid is provided at a dose of between about 1 mg/day and about 50 mg/day of prednisone-equivalent, between about 2.5 mg/day and about 5 mg/day of prednisone-equivalent, or between about 30 mg/day and about 40 mg/day of prednisone-equivalent. In some embodiments, the steroid is provided at a dose of no more than 20 mg/day.

In some embodiments, the additional therapeutic agent is an immunosuppressive agent. In some embodiments, the immunosuppressive agent comprises an antithymocyte globulin (ATG), an inhibitor of mammalian target of rapamycin (mTOR), a calcineurin inhibitor, or any combination thereof. In some embodiments, the immunosuppressive agent is an antithymocyte globulin (ATG). In some embodiments, the immunosuppressive agent is ATG-FRESENIUS®, ATGAM® or THYMOGLOBULIN®. In some embodiments, the immunosuppressive agent is ATG-FRESENIUS®. In some embodiments, the immunosuppressive agent is ATGAM®. In some embodiments, the immunosuppressive agent is THYMOGLOBULIN®. In some embodiments, the mTOR inhibitor is rapamycin (also known as sirolimus or RAPAMUNE®). In some embodiments, the immunosuppressive agent is a calcineurin inhibitor selected from the group consisting of tacrolimus (also known as FK506), cyclosporin A (also known as cyclosporine, ciclosporin, and CsA), and voclosporin (LUPKYNIS®). In some embodiments, the immunosuppressive agent is tacrolimus. In some embodiments, the immunosuppressive agent is cyclosporin A. In some embodiments, the immunosuppressive agent is voclosporin (LUPKYNIS®). In some embodiments, the immunosuppressive agent is selected from the group consisting of mycophenolate mofetil (CELLCEPT®), mycophenolic acid (MYFORTIC®), methotrexate, azathioprine, or any combination thereof. In some embodiments, the immunosuppressive agent is mycophenolate mofetil (CELLCEPT®). In some embodiments, the immunosuppressive agent is mycophenolic acid (MYFORTIC®). In some embodiments, the immunosuppressive agent is methotrexate. In some embodiments, the immunosuppressive agent is azathioprine. In some embodiments, the immunosuppressive agent is azathioprine (IMURAN®).

In some embodiments, the additional therapeutic agent is sulfasalazine.

In some embodiments, the additional therapeutic agent is an antimalarial agent. In some embodiments, the antimalarial agent comprises hydroxychloroquine (PLAQUENIL®), chloroquine (ARALEN®), quinacrine (ATABRINE®), or any combination thereof. In some embodiments, the antimalarial agent is hydroxychloroquine (PLAQUENIL®). In some embodiments, the antimalarial agent is chloroquine (ARALEN®). In some embodiments, the antimalarial agent is quinacrine (ATABRINE®).

In some embodiments, the additional therapeutic agent is an alkylating agent. In some embodiments, the alkylating agent is or comprises cyclophosphamide. In some embodiments, the alkylating agent is cyclophosphamide.

In some embodiments, the additional therapeutic agent is or comprises leflunomide. In some embodiments, the additional therapeutic agent comprises leflunomide. In some embodiments, the additional therapeutic agent is leflunomide.

In some embodiments, the additional therapeutic agent comprises a CTLA-4 fusion protein. In some embodiments, the immunosuppressive agent is a CTLA-4-IgG1 fusion protein. In some embodiments, the immunosuppressive agent comprises abatacept or belatacept. In some embodiments, the immunosuppressive agent comprises abatacept. In some embodiments, the immunosuppressive agent comprises belatacept.

In some embodiments, the additional therapeutic agent is a B cell-targeting agent. In some embodiments, the B cell-targeting agent is an antagonistic of CD19, CD20, CD22, and/or BAFF. In some embodiments, the B cell-targeting agent is an anti-BAFF antibody. In some embodiments, the B cell-targeting agent is belimumab (BENLYSTAR). In some embodiments, the B cell-targeting agent is an anti-CD19 antibody. In some embodiments, the B cell-targeting agent is an anti-CD20 antibody, such as rituximab (RITUXAN®) or ocrelizumab (OCREVUS®). In some embodiments, the B cell-targeting agent is rituximab (RITUXAN®). In some embodiments, the B cell-targeting agent is ocrelizumab. In some embodiments, the B cell-targeting agent is obinutuzumab (GAZYVA®). In some embodiments, the B cell-targeting agent is an anti-CD22 antibody. In some embodiments, the B cell-targeting agent is epratuzumab.

In some embodiments, the additional therapeutic agent is a nonsteroid anti-inflammatory drug (NSAID). In some embodiments, the NSAID comprises ibuprofen. In some embodiments, the NSAID comprises naproxen (e.g., naproxen sodium). In some embodiments, the NSAID comprises aspirin. In some embodiments, the NSAID comprises celecoxib (CELEBREX®). In some embodiments, the NSAID comprises diclofenac (VOLTAREN®). In some embodiments, the NSAID comprises fenoprofen (NALFON®). In some embodiments, the NSAID comprises indomethacin (INDOCIN®). In some embodiments, the NSAID comprises ketorolac (TORADOL®).

In some embodiments, the additional therapeutic agent inhibits IL-1. In some embodiments, the additional therapeutic agent is anakinra.

In some embodiments, the additional therapeutic agent inhibits IL-6. In some embodiments, the additional therapeutic agent is an anti-IL6 antibody. In some embodiments, the additional therapeutic agent is tocilizumab.

In some embodiments, the additional therapeutic agent inhibits TNF-alpha. In some embodiments, the additional therapeutic agent is an anti-TNF-alpha antibody. In some embodiments, the additional therapeutic agent is adalimumab.

In several embodiments, the additional therapeutic agent is an NK cell engager (e.g., a molecule that binds both an antigen expressed by target cells and an antigen expressed by NK cells). In several embodiments, the NK cell engager binds to an activating receptor on an NK cell and an antigen expressed by target cells. In some embodiments, the activating receptor on the NK cell is selected from the group consisting of CD16, NKp30, NKp46, NKG2D, and any combination thereof. In several embodiments, the NK cell engager binds to CD16. In several embodiments, the NK cell engager binds to NKp30. In several embodiments, the NK cell engager binds to NKp46. In several embodiments, the NK cell engager binds to NKG2D.

In several embodiments, the additional therapeutic agent is a plurality of immune cells (e.g., NK cells and/or T cells) engineered to express a CAR that binds to an antigen other than CD19. Accordingly, in some aspects, a method or use as provided herein comprises a combination of NK cells genetically engineered to express a CD19 CAR and a plurality of immune cells engineered to express a CAR that binds to an antigen other than CD19. The NK cells genetically engineered to express a CD19 CAR and the plurality of immune cells can be part of the same composition or can be provided as separate compositions (e.g., concurrently or at different times). For example, in some aspects, the additional therapeutic agent is a plurality of immune cells engineered to express a CAR that binds to an antigen selected from the group consisting of BAFF-R. BCMA, CD20, CD22, CD27, CD28, CD33, CD38, CD45, CD47, CD54, CD56, CD81, CD117, CD138, CD200, FcRH5, GPRC5D, and SLAMF7. In some embodiments, the antigen is BAFF-R. In some embodiments, the antigen is BCMA. In some embodiments, the antigen is CD20. In some embodiments, the antigen is CD22. In some embodiments, the antigen is CD27. In some embodiments, the antigen is CD28. In some embodiments, the antigen is CD33. In some embodiments, the antigen is CD38. In some embodiments, the antigen is CD45. In some embodiments, the antigen is CD47. In some embodiments, the antigen is CD54. In some embodiments, the antigen is CD56. In some embodiments, the antigen is CD81. In some embodiments, the antigen is CD117. In some embodiments, the antigen is CD138. In some embodiments, the antigen is CD200. In some embodiments, the antigen is FcRH5. In some embodiments, the antigen is GPRC5D. In some embodiments, the antigen is SLAMF7.

In some embodiments, the plurality of immune cells comprises NK cells. In some embodiments, the plurality of immune cells comprises T cells. In some embodiments, the plurality of immune cells comprises NK cells and T cells. In some embodiments, the plurality of immune cells are allogeneic to the subject. In some embodiments, the plurality of immune cells are autologous to the subject.

### V. Articles of Manufacture and Kits

Also provided are articles of manufacture or kits containing the provided genetically engineered NK cells and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Non-limiting examples of containers include intravenous solution bags and vials, including those with stoppers pierceable by a needle for injection. The article of manufacture or kit may further include a package insert indicating that the composition can be used to treat a particular condition such as a condition described herein (e.g., an autoimmune disease). Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating a B cell-mediated disease in an individual. The label or package insert may indicate that the composition is used for treating an autoimmune disease (e.g., SLE) in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing a B cell-mediated disease in an individual. The label or package insert may further indicate that the formulation is useful or intended for subcutaneous, intravenous, or other modes of administration for treating or preventing an autoimmune disease (e.g., SLE) in an individual.

The container in some embodiments holds a composition which is by itself or combined with another composition effective for treating, preventing and/or diagnosing the disease. The article of manufacture or kit may include (a) a first container with a composition contained therein (i.e., first medicament), wherein the composition includes the engineered NK cells; and (b) a second container with a composition contained therein (i.e., second medicament), wherein the composition includes a further agent, such as a cytotoxic or otherwise therapeutic agent, and which article or kit further comprises instructions on the label or package insert for treating the subject with the second medicament, in an effective amount.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The terms “full length antibody.” “intact antibody.” and “whole antibody” are used herein interchangeably to refer to an antibody having a structure substantially similar to a native antibody structure or having heavy chains that contain an Fe region as defined herein.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-CD19 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The terms “polypeptide” and “protein” are used interchangeably to refer to a polymer of amino acid residues, and are not limited to a minimum length. Polypeptides, including the antibodies and antibody chains and other peptides, e.g., linkers and CD19-binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into a binding molecule, e.g., antibody, of interest and the products screened for a desired activity, e.g., retained/improved antigen binding, or decreased immunogenicity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative amino acid substitutions will involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising.” “consisting.” and/or “consisting essentially of aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “rheumatoid arthritis” or “RA” refers to a recognized disease state that may be diagnosed according to the 2000 revised American Rheumatoid Association criteria for the classification of RA, or any similar criteria. In some embodiments, the term “rheumatoid arthritis” refers to a chronic autoimmune disease characterized primarily by inflammation of the lining (synovium) of the joints, which can lead to joint damage, resulting in chronic pain, loss of function, and disability. Because RA can affect multiple organs of the body, including skin, lungs, and eyes, it is referred to as a systemic illness.

The term “rheumatoid arthritis” includes not only active and early RA, but also incipient RA, as defined below. Physiological indicators of RA include, symmetric joint swelling which is characteristic though not invariable in RA. Fusiform swelling of the proximal interphalangeal (PIP) joints of the hands as well as metacarpophalangeal (MCP), wrists, elbows, knees, ankles, and metatarsophalangeal (MTP) joints are commonly affected and swelling is easily detected. Pain on passive motion is the most sensitive test for joint inflammation, and inflammation and structural deformity often limits the range of motion for the affected joint. Typical visible changes include ulnar deviation of the fingers at the MCP joints, hyperextension, or hyperflexion of the MCP and PIP joints, flexion contractures of the elbows, and subluxation of the carpal bones and toes. The subject with RA may be resistant to a disease-modifying anti-rheumatic drug (DMARD), and/or a non-steroidal anti-inflammatory drug (NSAID). Nonlimiting examples of “DMARDs” include hydroxycloroquine, sulfasalazine, methotrexate (MTX), leflunomide, etanercept, infliximab (plus oral and subcutaneous MTX), azathioprine, D-penicillamine, gold salts (oral), gold salts (intramuscular), minocycline, cyclosporine including cyclosporine A and topical cyclosporine, staphylococcal protein A (Goodyear and Silverman, J. Exp. Med., 197 (9): 1125-1139 (2003)), including salts and derivatives thereof, etc.

A patient with “active rheumatoid arthritis” means a patient with active and not latent symptoms of RA. Subjects with “early active rheumatoid arthritis” are those subjects with active RA diagnosed for at least 8 weeks but no longer than four years, according to the revised 1987 ACR criteria for the classification of RA.

Subjects with “early rheumatoid arthritis” are those subjects with RA diagnosed for at least eight weeks but no longer than four years, according to the revised 1987 ACR criteria for classification of RA. RA includes, for example, juvenile-onset RA, juvenile idiopathic arthritis (JIA), or juvenile RA (JRA).

Patients with “incipient RA” have early polyarthritis that does not fully meet ACR criteria for a diagnosis of RA, in association with the presence of RA-specific prognostic biomarkers such as anti-CCP and shared epitope. They include patients with positive anti-CCP antibodies who present with polyarthritis, but do not yet have a diagnosis of RA, and are at high risk for going on to develop bona fide ACR criteria RA (95% probability).

The term “lupus” as used herein is an autoimmune disease or disorder that in general involves antibodies that attack connective tissue. The principal form of lupus is a systemic one, systemic lupus erythematosus (SLE), including cutaneous SLE and subacute cutaneous SLE, as well as other types of lupus (including nephritis, extrarenal, cerebritis, pediatric, non-renal, discoid, and alopecia). In certain embodiments, the term “systemic lupus erythematosus” refers to a chronic autoimmune disease that can result in skin lesions, joint pain and swelling, kidney disease (lupus nephritis), fluid around the heart and/or lungs, inflammation of the heart, and various other systemic conditions. In certain embodiments, the term “lupus nephritis” refers to inflammation of the kidneys that occurs in patients with SLE. Lupus nephritis may include, for example, glomerulonephritis and/or interstitial nephritis, and can lead to hypertension, proteinuria, and kidney failure. Lupus nephritis may be classified based on severity and extent of disease, for example, as defined by the International Society of Nephrology/Renal/Pathology Society. Lupus nephritis classes include class I (minimal mesangial lupus nephritis), class II (mesangial proliferative lupus nephritis), class III (focal lupus nephritis), class IV (diffuse segmental (IV-S) or diffuse global (IV-G) lupus nephritis), class V (membranous lupus nephritis), and class VI (advanced sclerosing lupus nephritis). The term “lupus nephritis” encompasses all of the classes.

The term “multiple sclerosis” (“MS”) refers to the chronic and often disabling disease of the central nervous system characterized by the progressive destruction of the myelin. “Demyelination” occurs when the myelin sheath becomes inflamed, injured, and detaches from the nerve fiber. There are four internationally recognized forms of MS, namely, primary progressive multiple sclerosis (PPMS), relapsing-remitting multiple sclerosis (RRMS), secondary progressive multiple sclerosis (SPMS), and progressive relapsing multiple sclerosis (PRMS).

“Primary progressive multiple sclerosis” or “PPMS” is characterized by a gradual progression of the disease from its onset with no superimposed relapses and remissions at all. There may be periods of a leveling off of disease activity. PPMS differs from RRMS and SPMS in that onset is typically in the late thirties or early forties, men are as likely as women to develop it, and initial disease activity is often in the spinal cord and not in the brain. PPMS often migrates into the brain but is less likely to damage brain areas than RRMS or SPMS; for example, people with PPMS are less likely to develop cognitive problems. PPMS is the sub-type of MS that is least likely to show inflammatory (gadolinium enhancing) lesions on MRI scans. The primary progressive form of the disease affects between 10 and 15% of all people with multiple sclerosis. PPMS may be defined according to the criteria in McDonald et al. Ann Neurol 50:121-7 (2001). The subject with PPMS treated herein is usually one with a probable or definitive diagnosis of PPMS.

“Relapsing-remitting multiple sclerosis” or “RRMS” is characterized by relapses (also known as exacerbations) during which time new symptoms can appear and old ones resurface or worsen. The relapses are followed by periods of remission, during which time the person fully or partially recovers from the deficits acquired during the relapse. Relapses can last for days, weeks, or months, and recovery can be slow and gradual or almost instantaneous. The vast majority of people presenting with MS are first diagnosed with RRMS. This is typically when they are in their twenties or thirties, though diagnoses much earlier or later are known. Twice as many women as men present with this sub-type of MS. During relapses, myelin, a protective insulating sheath around the nerve fibers (neurons) in the white matter regions of the central nervous system (CNS), may be damaged in an inflammatory response by the body's own immune system. This causes a wide variety of neurological symptoms that vary considerably depending on which areas of the CNS are damaged. Immediately after a relapse, the inflammatory response dies down and oligodendrocytes sponsor remyelination—a process whereby the myelin sheath around the axon may be repaired. It is this remyelination that may be responsible for the remission. Approximately 50% of patients with RRMS convert to SPMS within 10 years of disease onset. After 30 years, this figure rises to 90%. At any one time, the relapsing-remitting form of the disease accounts around 55% of all people with MS.

“Secondary progressive multiple sclerosis” or “SPMS” is characterized by a steady progression of clinical neurological damage with or without superimposed relapses and minor remissions and plateau. People who develop SPMS will have previously experienced a period of RRMS which may have lasted anywhere from two to forty years or more. Any superimposed relapses and remissions tend to tail off over time. SPMS tends to be associated with lower levels of inflammatory lesion formation than in RRMS but the total burden of disease continues to progress. At any one time, SPMS accounts around 30% of all people with multiple sclerosis.

“Progressive relapsing multiple sclerosis” or “PRMS” is characterized by a steady progression of clinical neurological damage with superimposed relapses and remissions. There is significant recovery immediately following a relapse but between relapses there is a gradual worsening of symptoms. PRMS affects around 5% of all people with multiple sclerosis.

### Non-Limiting Embodiments

Among the embodiments provided herein are:

1. A method of treating systemic lupus erythematosus (SLE), the method comprising administering to a subject having SLE a composition comprising natural killer (NK) cells genetically engineered to express a chimeric antigen receptor (CAR) that binds to CD19, wherein the CAR comprises:
(a) an extracellular antigen-binding domain;
(b) a transmembrane domain; and
(c) an intracellular signaling domain.

2. A method of treating lupus nephritis (LN), the method comprising administering to a subject having LN a composition comprising natural killer (NK) cells genetically engineered to express a chimeric antigen receptor (CAR) that binds to CD19, wherein the CAR comprises:
(a) an extracellular antigen-binding domain;
(b) a transmembrane domain; and
(c) an intracellular signaling domain.

3. The method of embodiment 1 or embodiment 2, wherein the extracellular antigen-binding domain comprises a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively.

4. A method of reducing B cells in a subject, the method comprising administering to a subject having an autoimmune disease a composition comprising natural killer (NK) cells genetically engineered to express a chimeric antigen receptor (CAR) that binds to CD19, wherein the genetically engineered NK cells are allogeneic to the subject.

5. A method of treating an autoimmune disease, the method comprising administering to a subject having an autoimmune disease a composition comprising natural killer (NK) cells genetically engineered to express a chimeric antigen receptor (CAR) that binds to CD19, wherein the CAR comprises:
(a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively;
(b) a transmembrane domain comprising a CD8alpha transmembrane region; and
(c) an intracellular signaling domain comprising an intracellular signaling region of OX40 and a CD3zeta domain.

6. A method of treating an autoimmune disease, the method comprising administering to a subject having an autoimmune disease a composition comprising natural killer (NK) cells genetically engineered to express a chimeric antigen receptor (CAR) that binds to CD19, wherein the genetically engineered NK cells are allogeneic to the subject.

7. The method of embodiment 4 or embodiment 6, wherein the CAR comprises (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively; (b) a transmembrane domain; and (c) an intracellular signaling domain.

8. The method of any one of embodiments 4-7, wherein the autoimmune disease is systemic lupus erythematosus (SLE).

9. The method of any one of embodiments 4-8, wherein the autoimmune disease is or comprises lupus nephritis (LN).

10. The method of any one of embodiments 4-7, wherein the autoimmune disease comprises idiopathic inflammatory myopathy (IIM), multiple sclerosis (MS), myasthenia gravis (MG), rheumatoid arthritis (RA), scleroderma, thyroid disease, type 1 diabetes, vasculitis, or any combination thereof.

11. The method of any one of embodiments 4-7 and 10, wherein the autoimmune disease comprises IIM, optionally wherein the autoimmune disease comprises anti-synthetase syndrome.

12. The method of any one of embodiments 4-7 and 10, wherein the autoimmune disease comprises MS, optionally wherein the autoimmune disease comprises primary progressive MS (PPMS).

13. The method of any one of embodiments 4-7 and 10, wherein the autoimmune disease comprises MG.

14. The method of any one of embodiments 4-7 and 13, wherein the autoimmune disease comprises RA, optionally wherein the autoimmune disease comprises RA that is refractory to a tumor necrosis factor (TNF) inhibitor.

15. The method of any one of embodiments 4-7 and 10, wherein the autoimmune disease comprises scleroderma, optionally wherein the autoimmune disease comprises systemic scleroderma (SSc).

16. The method of any one of embodiments 4-7 and 10, wherein the autoimmune disease comprises thyroid disease.

17. The method of any one of embodiments 4-7 and 10, wherein the autoimmune disease comprises type 1 diabetes.

187. The method of any one of embodiments 4-7 and 10, wherein the autoimmune disease comprises vasculitis, optionally wherein the autoimmune disease comprises ANCA-associated vasculitis (AAV).

19. A method of treating systemic lupus erythematosus (SLE), the method comprising administering to a subject having SLE a composition comprising natural killer (NK) cells genetically engineered to express a chimeric antigen receptor (CAR) that binds to CD19, wherein the CAR comprises:

(a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively;
(b) a transmembrane domain; and
(c) an intracellular signaling domain.

20. A method of treating lupus nephritis (LN), the method comprising administering to a subject having LN a composition comprising natural killer (NK) cells genetically engineered to express a chimeric antigen receptor (CAR) that binds to CD19, wherein the CAR comprises:
(a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively;
(b) a transmembrane domain; and
(c) an intracellular signaling domain.

21. The method of any one of embodiments 1-3 and 7-20, wherein the transmembrane domain comprises a CD8alpha transmembrane region, and the intracellular signaling domain comprises an intracellular signaling region of OX40 and a CD3zeta domain.

22. The method of any one of embodiments 3, 5, and 7-21, wherein the VH comprises the amino acid sequence set forth in SEQ ID NO:35, and the VL comprises the amino acid sequence set forth in SEQ ID NO:36.

23. The method of any one of embodiments 1-3, 5 and 7-22, wherein the extracellular antigen-binding domain is a single-chain variable fragment (scFv) comprising the amino acid sequence set forth in SEQ ID NO:37.

24. The method of any one of embodiments 1-3, 5 and 7-23, wherein the transmembrane domain comprises the amino acid sequence set forth in SEQ ID NO:8.

25. The method of any one of embodiments 5, 8-18, and 21-24, wherein the intracellular signaling region of OX40 comprises the amino acid sequence set forth in SEQ ID NO: 14.

26. The method of any one of embodiments 5, 8-18, and 21-25, wherein the CD3zeta domain comprises the amino acid sequence set forth in SEQ ID NO:16.

27. The method of any one of embodiments 1-26, wherein the CAR comprises the amino acid sequence set forth in SEQ ID NO:38.

28. The method of any one of embodiments 1-27, wherein the NK cells genetically engineered to express a CAR also express a membrane-bound interleukin-15 (mbIL15).

29. The method of embodiment 28, wherein the mbIL15 comprises the amino acid sequence set forth in SEQ ID NO:23.

30. The method of embodiment 28, wherein the mbIL15 comprises the amino acid sequence set forth in SEQ ID NO:40.

31. The method of any one of embodiments 28-30, wherein the CAR and the mbIL15 are bicistronically encoded by the same nucleic acid molecule, optionally wherein the nucleic acid sequences encoding the CAR and the mbIL15 are separated by a nucleic acid sequence encoding a T2A peptide.

32. The method of any one of embodiments 1-31, wherein the composition comprising NK cells genetically engineered to express a CAR is administered to the subject in a dosing regimen comprising a dosing cycle.

33. The method of embodiment 32, wherein the dosing cycle comprises a first dose, a second dose, and a third dose of the composition comprising NK cells genetically engineered to express a CAR.

34. The method of embodiment 33, wherein the second dose is administered to the subject between about 5 days after and about 10 days after the first dose is administered to the subject.

35. The method of embodiment 33 or embodiment 34, wherein the third dose is administered to the subject between about 5 days after and about 10 days after the second dose is administered to the subject.

36. The method of any one of embodiments 33-35, wherein the second dose is administered about 7 days after the first dose is administered to the subject, and the third dose is administered about 7 days after the second dose is administered to the subject.

37. The method of embodiment 33, wherein the second dose is administered to the subject between about 2 days after and about 4 days after the first dose is administered to the subject.

28. The method of embodiment 33 or embodiment 37, wherein the third dose is administered to the subject between about 2 days after and about 4 days after the second dose is administered to the subject.

39. The method of any one of embodiments 33, 37, and 38, wherein the second dose is administered about 3 days after the first dose is administered to the subject, and the third dose is administered about 4 days after the second dose is administered to the subject.

40. The method of any one of embodiments 32-39, wherein the dosing cycle is between about 14 days and about 35 days, or between about 21 days and about 28 days, each inclusive.

41. The method of any one of embodiments 32-40, wherein the dosing cycle is about 28 days.

42. The method of any one of embodiments 32-39, wherein the dosing cycle is between about 35 days and about 42 days, each inclusive.

43. The method of any one of embodiments 32-39 and 42, wherein the dosing cycle is about 42 days.

44. The method of any one of embodiments 33-43, wherein the first dose is administered on about Day 0 of the dosing cycle, the second dose is administered on about Day 7 of the dosing cycle, and the third dose is administered on about Day 14 of the dosing cycle.

45. The method of any one of embodiments 33 and 37-43, wherein the first dose is administered on about Day 0 of the dosing cycle, the second dose is administered on about Day 3 of the dosing cycle, and the third dose is administered on about Day 7 of the dosing cycle.

46. The method of any one of embodiments 33-45, wherein each dose of the dosing cycle comprises between about 1×10⁸ CAR-expressing NK cells and about 1×10¹⁰ CAR-expressing NK cells, or between about 3×10⁸ CAR-expressing NK cells and about 3×10⁹ CAR-expressing NK cells, each inclusive.

47. The method of any one of embodiments 33-46, wherein each dose of the dosing cycle comprises about 3×10⁸ CAR-expressing NK cells, about 1×10⁹ CAR-expressing NK cells, or about 1.5×10⁹ CAR-expressing NK cells.

48. The method of any one of embodiments 33-46, wherein each dose of the dosing cycle comprises about 2×10⁹ CAR-expressing NK cells or about 2.5×10⁹ CAR-expressing NK cells.

49. A method of treating systemic lupus erythematosus (SLE), the method comprising administering to a subject having SLE a composition comprising natural killer (NK) cells genetically engineered to express a chimeric antigen receptor (CAR) that binds to CD19, wherein:
(i) the CAR comprises:
   (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO: 36;
   (b) a transmembrane domain comprising a CD8alpha transmembrane region; and
   (c) an intracellular signaling domain comprising an intracellular signaling region of OX40 and a CD3zeta domain;
(ii) the composition comprising the NK cells genetically engineered to express a CAR is administered to the subject in a dosing regimen comprising a dosing cycle, wherein the dosing cycle comprises a first dose, a second dose, and a third dose of the composition;
(iii) each of the first, second, and third doses of the dosing cycle comprises between about 1×10⁸ CAR-expressing NK cells and 2×10⁹ CAR-expressing NK cells; and
(iv) the second dose is administered to the subject about 7 days after the first dose is administered to the subject, and the third dose is administered to the subject about 7 days after the second dose is administered to the subject.

50. A method of treating lupus nephritis (LN), the method comprising administering to a subject having LN a composition comprising natural killer (NK) cells genetically engineered to express a chimeric antigen receptor (CAR) that binds to CD19, wherein:
(i) the CAR comprises:
   (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO: 36;
   (b) a transmembrane domain comprising a CD8alpha transmembrane region; and
   (c) an intracellular signaling domain comprising an intracellular signaling region of OX40 and a CD3zeta domain;
(ii) the composition comprising the NK cells genetically engineered to express a CAR is administered to the subject in a dosing regimen comprising a dosing cycle, wherein the dosing cycle comprises a first dose, a second dose, and a third dose of the composition;
(iii) each of the first, second, and third doses of the dosing cycle comprises between about 1×10⁸ CAR-expressing NK cells and 2×10⁹ CAR-expressing NK cells; and
   (iv) the second dose is administered to the subject about 7 days after the first dose is administered to the subject, and the third dose is administered to the subject about 7 days after the second dose is administered to the subject

51. A method of treating systemic lupus erythematosus (SLE), the method comprising administering to a subject having SLE a composition comprising natural killer (NK) cells genetically engineered to express a chimeric antigen receptor (CAR) that binds to CD19, wherein:
(i) the CAR comprises:
   (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO: 36;
   (b) a transmembrane domain comprising a CD8alpha transmembrane region; and
   (c) an intracellular signaling domain comprising an intracellular signaling region of OX40 and a CD3zeta domain;
(ii) the composition comprising the NK cells genetically engineered to express a CAR is administered to the subject in a dosing regimen comprising a dosing cycle, wherein the dosing cycle comprises a first dose, a second dose, and a third dose of the composition;
(iii) each of the first, second, and third doses of the dosing cycle comprises about 2×10⁹ CAR-expressing NK cells or about 2×10⁹ CAR-expressing NK cells; and
(iv) the second dose is administered to the subject about 3 days after the first dose is administered to the subject, and the third dose is administered to the subject about 4 days after the second dose is administered to the subject.

52. A method of treating lupus nephritis (LN), the method comprising administering to a subject having LN a composition comprising natural killer (NK) cells genetically engineered to express a chimeric antigen receptor (CAR) that binds to CD19, wherein:
(i) the CAR comprises:
   (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO: 36;
   (b) a transmembrane domain comprising a CD8alpha transmembrane region; and
   (c) an intracellular signaling domain comprising an intracellular signaling region of OX40 and a CD3zeta domain;
(ii) the composition comprising the NK cells genetically engineered to express a CAR is administered to the subject in a dosing regimen comprising a dosing cycle, wherein the dosing cycle comprises a first dose, a second dose, and a third dose of the composition;
(iii) each of the first, second, and third doses of the dosing cycle comprises about 2×10⁹ CAR-expressing NK cells or about 2.5×10⁹ CAR-expressing NK cells; and
   (iv) the second dose is administered to the subject about 3 days after the first dose is administered to the subject, and the third dose is administered to the subject about 4 days after the second dose is administered to the subject.

53. The method of any one of embodiments 1-52, wherein the NK cells genetically engineered to express a CAR are allogeneic to the subject.

54. The method of any one of embodiments 1-53, wherein:
(i) the method comprises administering a lymphodepleting therapy to the subject prior to administration of the composition comprising NK cells genetically engineered to express a CAR; and/or
(ii) prior to administration of the composition comprising NK cells genetically engineered to express a CAR to the subject, the subject has been administered a lymphodepleting therapy.

55. The method of embodiment 54, wherein the lymphodepleting therapy comprises administration of cyclophosphamide.

56. A method of treating or preventing an autoimmune disease, the method comprising administering to a subject having or suspected or having, or determined to be at risk of, an autoimmune disease a composition comprising natural killer (NK) cells genetically engineered to express a chimeric antigen receptor (CAR) that binds to CD19, wherein:
(i) the CAR comprises:
   (a) an extracellular antigen-binding domain;
   (b) a transmembrane domain; and
   (c) an intracellular signaling domain,
(ii) the composition comprising the NK cells genetically engineered to express a CAR is administered to the subject in a dosing regimen comprising a dosing cycle;
(iii) prior to administration of the composition comprising NK cells genetically engineered to express a CAR to the subject, the subject has been administered a lymphodepleting therapy; and
(iv) the lymphodepleting therapy comprises administration of cyclophosphamide and does not comprise administration of fludarabine.

57. The method of any one of embodiments 54-56, wherein the lymphodepleting therapy comprises administration of cyclophosphamide at between about 500 mg/m² and about 1500 mg/m², optionally wherein the lymphodepleting therapy comprises administration of a single dose of about 1000 mg/m² cyclophosphamide.

58. The method of any one of embodiments 54, 55, and 57, wherein the lymphodepleting therapy comprises administration of fludarabine.

59. The method of any one of embodiments 54-58, wherein the lymphodepleting therapy comprises administration of (i) cyclophosphamide at between about 200 and about 600 mg/m² per day, optionally at about 500 mg/m² per day, daily for 2-4 days, optionally for 3 days; and/or (ii) fludarabine at between about 20 and about 40 mg/m² per day, optionally at about 30 mg/m², daily for 2-4 days, optionally for 3 days.

60. The method of any one of embodiments 54, 55, 57, and 59, wherein the lymphodepleting therapy comprises administration of fludarabine at between about 20 and about 40 mg/m² per day, optionally at about 25 mg/m², daily for 3 days.

61. The method of any one of embodiments 54-60, wherein the lymphodepleting therapy comprises administration of about 500 mg/m² of cyclophosphamide on each of Days −3, −4, and −5.

62. The method of any one of embodiments 54, 55, 57, and 59-61, wherein the lymphodepleting therapy comprises administration of about 500 mg/m² of cyclophosphamide and about 30 mg/m² of fludarabine on each of Days −5, −4, and −3.

63. The method of any one of embodiments 54-61, wherein the lymphodepleting therapy comprises administration of about 1000 mg/m² of cyclophosphamide on Day-3.

64. The method of any one of embodiments 54-63, wherein the subject is administered a corticosteroid before, during, and/or after administration of the lymphodepleting therapy, optionally wherein the corticosteroid comprises a glucocorticoid.

65. The method of any one of embodiments 1-64, wherein the subject is administered a corticosteroid before, during, and/or after administration of the composition, optionally wherein the corticosteroid comprises a glucocorticoid.

66. The method of any one of embodiments 1-65, wherein the subject is administered an immunosuppressive agent before, during, and/or after administration of the lymphodepleting therapy.

67. The method of any one of embodiments 1-66, wherein the subject is administered an immunosuppressive agent before, during, and/or after administration of the composition.

68. The method of embodiment 66 or embodiment 67, wherein the immunosuppressive agent comprises an antithymocyte globulin (ATG), an inhibitor of mammalian target of rapamycin (mTOR), a calcineurin inhibitor, or any combination thereof.

69. The method of any one of embodiments 1, 3, 8, 9, 19, 21-49, 51, 53-55, and 57-68, wherein the subject has relapsed following treatment with and/or is refractory to a prior line of therapy for the SLE.

70. The method of any one of embodiments 2, 3, 9, 20-48, 50, 52-55, and 57-68, wherein the subject has relapsed following treatment with and/or is refractory to a prior line of therapy for the LN.

71. The method of any one of embodiments 4-18, 21-48, and 53-68, wherein the subject has relapsed following treatment with and/or is refractory to a prior line of therapy for the autoimmune disease.

72. The method of any one of embodiments 69-71, wherein the prior line of therapy comprises two, three, or four prior lines of therapy.

73. The method of any one of embodiments 69-72, wherein the prior line of therapy comprises a corticosteroid, an immunosuppressive agent, an antimalarial agent, a B cell-targeting agent, hematopoietic stem cell transplant (HSCT), or any combination thereof.

74. The method of any one of embodiments 1-73, wherein the subject does not have lupus nephritis (LN) and/or does not have CNS lupus.

75. The method of any one of embodiments 1-74, wherein the subject does not have CNS lupus.

76. The method of any one of embodiments 32-75, wherein the dosing regimen comprises or consists of two, three, four, or five dosing cycles.

77. The method of any one of embodiments 1-76, wherein, among a plurality of subjects treated according to the method, the number of peripheral B cells in the subjects is reduced by an average of at least about 70%, at least about 80%, at least about 90%, at least about 95%, or at least about 99%, optionally as compared to subjects not treated according to the method.

78. A method of reducing B cells in a subject having a B cell-mediated disease comprising administering to the subject a composition comprising natural killer (NK) cells genetically engineered to express a chimeric antigen receptor (CAR) that binds to CD19, wherein:
(i) the composition comprising the NK cells genetically engineered to express a CAR is administered to the subject in a dosing regimen comprising a dosing cycle; and
(ii) the method reduces peripheral B cells in the subject by at least about 90%; peripheral B cells are significantly reduced in the subject for the duration of the dosing cycle; and/or at least about 75% of repopulating peripheral B cells are non-class-switched B cells.

79. The method of any one of embodiments 1-78, wherein, among a plurality of subjects treated according to the method, the number of peripheral B cells in the subjects is significantly reduced within about 10 days, within about 15 days, or within about 30 days after administration of a first dose of the composition comprising NK cells genetically engineered to express a CAR to the subjects, optionally as compared to subjects not treated according to the method.

80. The method of any one of embodiments 1-79, wherein, among a plurality of subjects treated according to the method, the number of peripheral B cells in the subjects is significantly reduced for at least about 15 days, at least about 1 month, at least about 2 months, at least about 3 months, at least about 6 months, or at least about 9 months following a final dose of the composition comprising NK cells genetically engineered to express a CAR, optionally as compared to subjects not treated according to the method.

81. The method of any one of embodiments 1-80, wherein, at about 3 months, at about 6 months, at about 9 months, and/or at about 12 months after administration of a final dose of the composition comprising NK cells genetically engineered to express a CAR to the subject, at least about 50%, at least about 60%, at least about 70%, or at least about 80% of the peripheral B cells in the subject are naïve B cells.

82. The method of embodiment 81, wherein the naïve B cells are non-class-switched, optionally wherein the naïve B cells are IgM or IgD isotype.

83. A method of preparing a subject having an autoimmune disease for treatment with a composition comprising natural killer (NK) cells genetically engineered to express a chimeric antigen receptor (CAR) that binds to CD19, the method comprising administering a lymphodepleting therapy to the subject prior to administration of the composition to the subject, wherein the lymphodepleting therapy consists of cyclophosphamide.

84. The method of embodiment 83, wherein the lymphodepleting therapy comprises cyclophosphamide at between about 500 mg/m² and about 1500 mg/m², optionally wherein the lymphodepleting therapy comprises a single dose of about 1000 mg/m² cyclophosphamide.

85. The method of embodiment 83 or embodiment 84, wherein the lymphodepleting therapy comprises a single dose of about 1000 mg/m² cyclophosphamide about 3 days before administration of the composition.

86. The method of any one of embodiments 83-85, wherein the NK cells genetically engineered to express a CAR that binds to CD19 also express a membrane-bound interleukin-15 (mbIL15).

87. Use of a composition comprising natural killer (NK) cells genetically engineered to express a chimeric antigen receptor (CAR) that binds to CD19 for treating a subject with systemic lupus erythematosus (SLE), wherein the CAR comprises:
(a) an extracellular antigen-binding domain;
(b) a transmembrane domain; and
(c) an intracellular signaling domain.

88. Use of a composition comprising natural killer (NK) cells genetically engineered to express a chimeric antigen receptor (CAR) that binds to CD19 for treating a subject with lupus nephritis (LN), wherein the CAR comprises:
(a) an extracellular antigen-binding domain;
(b) a transmembrane domain; and
(c) an intracellular signaling domain.

89. Use of a composition comprising natural killer (NK) cells genetically engineered to express a chimeric antigen receptor (CAR) that binds to CD19 for reducing B cells in a subject having an autoimmune disease, wherein the NK cells are allogeneic to the subject.

90. Use of a composition comprising natural killer (NK) cells genetically engineered to express a chimeric antigen receptor (CAR) that binds to CD19 for treating a subject having an autoimmune disease, wherein the genetically engineered NK cells are allogeneic to the subject.

91. The method or use of any one of embodiments 78-90, wherein the CAR comprises: (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively; (b) a transmembrane domain comprising a CD8alpha transmembrane region; and (c) an intracellular signaling domain comprising an intracellular signaling region of OX40 and a CD3zeta domain.

92. Use of a composition comprising natural killer (NK) cells genetically engineered to express a chimeric antigen receptor (CAR) that binds to CD19 for treating a subject having an autoimmune disease, wherein the CAR comprises:
(a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively;
(b) a transmembrane domain comprising a CD8alpha transmembrane region; and
(c) an intracellular signaling domain comprising an intracellular signaling region of OX40 and a CD3zeta domain.

93. The use of any one of embodiments 89-92, wherein the autoimmune disease comprises idiopathic inflammatory myopathy (IIM), multiple sclerosis (MS), myasthenia gravis (MG), rheumatoid arthritis (RA), scleroderma, thyroid disease, type 1 diabetes, vasculitis, or any combination thereof.

94. Use of a composition comprising natural killer (NK) cells genetically engineered to express a chimeric antigen receptor (CAR) that binds to CD19 for treating a subject with systemic lupus erythematosus (SLE), wherein the CAR comprises:
(a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively;
(b) a transmembrane domain; and
(c) an intracellular signaling domain.

95. Use of a composition comprising natural killer (NK) cells genetically engineered to express a chimeric antigen receptor (CAR) that binds to CD19 for treating a subject with lupus nephritis (LN), wherein the CAR comprises:
(a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively;
(b) a transmembrane domain; and
   (c) an intracellular signaling domain.

96. Use of a composition comprising natural killer (NK) cells genetically engineered to express a chimeric antigen receptor (CAR) that binds to CD19 for treating a subject having systemic lupus erythematosus (SLE), wherein:
(i) the CAR comprises:
   (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO: 36;
   (b) a transmembrane domain comprising a CD8alpha transmembrane region; and
   (c) an intracellular signaling domain comprising an intracellular signaling region of OX40 and a CD3zeta domain;
(ii) the composition comprising the NK cells genetically engineered to express a CAR is formulated for administration in a dosing regimen comprising a dosing cycle, wherein the dosing cycle comprises a first dose, a second dose, and a third dose of the composition;
(iii) each of the first, second, and third doses of the dosing cycle comprises between about 1×10⁸ CAR-expressing NK cells and 2×10⁹ CAR-expressing NK cells; and
(iv) the second dose is for administration to the subject about 7 days after the first dose is administered to the subject, and the third dose is for administration to the subject about 7 days after the second dose is administered to the subject.

97. Use of a composition comprising natural killer (NK) cells genetically engineered to express a chimeric antigen receptor (CAR) that binds to CD19 for treating a subject having lupus nephritis (LN), wherein:
(i) the CAR comprises:
   (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO: 36;
   (b) a transmembrane domain comprising a CD8alpha transmembrane region; and
   (c) an intracellular signaling domain comprising an intracellular signaling region of OX40 and a CD3zeta domain;
(ii) the composition comprising the NK cells genetically engineered to express a CAR is formulated for administration in a dosing regimen comprising a dosing cycle, wherein the dosing cycle comprises a first dose, a second dose, and a third dose of the composition;
(iii) each of the first, second, and third doses of the dosing cycle comprises between about 1×10⁸ CAR-expressing NK cells and 3×10⁹ CAR-expressing NK cells; and
(iv) the second dose is for administration to the subject about 7 days after the first dose is administered to the subject, and the third dose is for administration to the subject about 7 days after the second dose is administered to the subject.

98. Use of a composition comprising natural killer (NK) cells genetically engineered to express a chimeric antigen receptor (CAR) that binds to CD19 for treating a subject having systemic lupus erythematosus (SLE), wherein:
(i) the CAR comprises:
   (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO: 36;
   (b) a transmembrane domain comprising a CD8alpha transmembrane region; and
   (c) an intracellular signaling domain comprising an intracellular signaling region of OX40 and a CD3zeta domain;
(ii) the composition comprising the NK cells genetically engineered to express a CAR is formulated for administration in a dosing regimen comprising a dosing cycle, wherein the dosing cycle comprises a first dose, a second dose, and a third dose of the composition;
(iii) each of the first, second, and third doses of the dosing cycle comprises about 2×10⁹ CAR-expressing NK cells or about 2.5×10⁹ CAR-expressing NK cells; and
(iv) the second dose is for administration to the subject about 3 days after the first dose is administered to the subject, and the third dose is for administration to the subject about 4 days after the second dose is administered to the subject.

99. Use of a composition comprising natural killer (NK) cells genetically engineered to express a chimeric antigen receptor (CAR) that binds to CD19 for treating a subject having lupus nephritis (LN), wherein:
(i) the CAR comprises:
   (a) an extracellular antigen-binding domain comprising a heavy chain variable region (VH) comprising the amino acid sequence set forth in SEQ ID NO:35, and a light chain variable region (VL) comprising the amino acid sequence set forth in SEQ ID NO: 36;
   (b) a transmembrane domain comprising a CD8alpha transmembrane region; and
   (c) an intracellular signaling domain comprising an intracellular signaling region of OX40 and a CD3zeta domain;
(ii) the composition comprising the NK cells genetically engineered to express a CAR is formulated for administration in a dosing regimen comprising a dosing cycle, wherein the dosing cycle comprises a first dose, a second dose, and a third dose of the composition;
(iii) each of the first, second, and third doses of the dosing cycle comprises about 2×10⁹ CAR-expressing NK cells or about 2.5×10⁹ CAR-expressing NK cells; and
(iv) the second dose is for administration to the subject about 3 days after the first dose is administered to the subject, and the third dose is for administration to the subject about 4 days after the second dose is administered to the subject.

100. Use of a composition comprising natural killer (NK) cells genetically engineered to express a chimeric antigen receptor (CAR) that binds to CD19 for reducing B cells in a subject having or suspected or having, or determined to be at risk of, an autoimmune disease, wherein:
(i) the CAR comprises:
   (a) an extracellular antigen-binding domain;
   (b) a transmembrane domain; and
   (c) an intracellular signaling domain,
(ii) prior to administration of the composition comprising NK cells genetically engineered to express a CAR to the subject, the subject has been administered a lymphodepleting therapy; and
(iii) the lymphodepleting therapy comprises administration of cyclophosphamide and does not comprise administration of fludarabine.

101. Use of a composition comprising natural killer (NK) cells genetically engineered to express a chimeric antigen receptor (CAR) that binds to CD19 for treating or preventing an autoimmune disease in a subject having or suspected or having, or determined to be at risk of, an autoimmune disease, wherein:
(i) the CAR comprises:
   (a) an extracellular antigen-binding domain;
   (b) a transmembrane domain; and
   (c) an intracellular signaling domain,
(ii) prior to administration of the composition comprising NK cells genetically engineered to express a CAR to the subject, the subject has been administered a lymphodepleting therapy; and
(iii) the lymphodepleting therapy comprises administration of cyclophosphamide and does not comprise administration of fludarabine.

102. The use of embodiment 100 or embodiment 101, wherein:
the extracellular antigen-binding domain comprises a heavy chain variable region (VH) comprising a CDR-1, a CDR-2, and a CDR-3 comprising the amino acid sequences set forth in SEQ ID NOS: 24, 25, and 26, respectively; and a light chain variable region (VL) comprising a CDR-1, a CDR-2, and a CDR-3 comprising the amino acid sequences set forth in SEQ ID NOS: 27, 28, and 29, respectively;
the VH comprises the amino acid sequence set forth in SEQ ID NO:35 and the VL comprises the amino acid sequence set forth in SEQ ID NO:36;
the transmembrane domain comprises a CD8alpha transmembrane region; and/or
the intracellular signaling domain comprises an intracellular signaling region of OX40 and a CD3zeta domain.

103. Use of a composition comprising natural killer (NK) cells genetically engineered to express a chimeric antigen receptor (CAR) that binds to CD19 for reducing peripheral B cells in a subject having a B cell-mediated disease wherein:
(i) the composition comprising the NK cells genetically engineered to express a CAR is for administration to the subject in a dosing regimen comprising a dosing cycle; and
(iii) peripheral B cells are reduced in the subject by at least about 90%; peripheral B cells are significantly reduced in the subject for the duration of the dosing cycle; and/or at least about 75% of repopulating peripheral B cells are non-class-switched B cells.

104. The use of any one of embodiments 87, 88, and 92-103, wherein the genetically engineered NK cells are allogeneic to the subject.

105. The use of any one of embodiments 87-104, wherein, among a plurality of subjects treated with the composition comprising NK cells genetically engineered to express a CAR, the number of peripheral B cells in the subjects is reduced by an average of at least about 70%, at least about 80%, at least about 90%, at least about 95%, or at least about 99%.

106. The use of any one of embodiments 87-105, wherein, among a plurality of subjects treated with the composition comprising NK cells genetically engineered to express a CAR, the number of peripheral B cells in the subjects is significantly reduced for at least about 15 days, at least about 1 month, at least about 2 months, at least about 3 months, at least about 6 months, or at least about 9 months following a final dose of the composition comprising NK cells genetically engineered to express a CAR.

107. Use of a lymphodepleting therapy for the preparation of a subject having an autoimmune disease for treatment with a composition comprising natural killer (NK) cells genetically engineered to express a chimeric antigen receptor (CAR) that binds to CD19, wherein: (i) the lymphodepleting therapy is administered to the subject prior to administration of the composition to the subject; and (ii) the lymphodepleting therapy comprises cyclophosphamide and does not comprise fludarabine.

108. The use of embodiment 107, wherein the lymphodepleting therapy comprises cyclophosphamide at between about 500 mg/m² and about 1500 mg/m², optionally wherein the lymphodepleting therapy comprises a single dose of about 1000 mg/m² cyclophosphamide.

109. The use of embodiment 107 or embodiment 108, wherein the lymphodepleting therapy comprises about 1000 mg/m² of cyclophosphamide about 3 days before administration of the composition.

110. The use of any one of embodiments 107-109, wherein the NK cells genetically engineered to express a CAR that binds to CD19 also express a membrane-bound interleukin-15 (mbIL15).

111. A kit comprising (i) a composition comprising natural killer (NK) cells genetically engineered to express a chimeric antigen receptor (CAR) that binds to CD19; and (ii) instructions for administering the composition to a subject having an autoimmune disease,
wherein the CAR comprises (a) an extracellular antigen-binding domain comprising a heavy chain variable region (VH) comprising a CDR-1, a CDR-2, and a CDR-3 comprising the amino acid sequences set forth in SEQ ID NOS: 24, 25, and 26, respectively; and a light chain variable region (VL) having a CDR-1, a CDR-2, and a CDR-3 comprising the amino acid sequences set forth in SEQ ID NOS: 27, 28, and 29, respectively; (b) a transmembrane domain comprising a CD8alpha transmembrane region; and (c) an intracellular signaling domain comprising an intracellular signaling region of OX40 and a CD3zeta domain.

112. A method of treating systemic lupus erythematosus (SLE), the method comprising administering to a subject having SLE a composition comprising natural killer (NK) cells genetically engineered to express a chimeric antigen receptor (CAR) that binds to CD19, wherein:
(i) prior to administration of the composition comprising NK cells genetically engineered to express a CAR to the subject, the subject has been administered a lymphodepleting therapy; and
(ii) the lymphodepleting therapy comprises administration of cyclophosphamide and does not comprise administration of fludarabine.

113. A method of treating lupus nephritis (LN), the method comprising administering to a subject having LN a composition comprising natural killer (NK) cells genetically engineered to express a chimeric antigen receptor (CAR) that binds to CD19, wherein:
(i) prior to administration of the composition comprising NK cells genetically engineered to express a CAR to the subject, the subject has been administered a lymphodepleting therapy; and
(ii) the lymphodepleting therapy comprises administration of cyclophosphamide and does not comprise administration of fludarabine.

114. A method of treating an autoimmune disease, the method comprising administering to a subject having an autoimmune disease a composition comprising natural killer (NK) cells genetically engineered to express a chimeric antigen receptor (CAR) that binds to CD19, wherein:
(i) prior to administration of the composition comprising NK cells genetically engineered to express a CAR to the subject, the subject has been administered a lymphodepleting therapy; and
(ii) the lymphodepleting therapy comprises administration of cyclophosphamide and does not comprise administration of fludarabine; and
(iii) the autoimmune disease is selected from the group consisting of scleroderma, myositis, and vasculitis.

115. A method of treating an autoimmune disease, the method comprising administering to a subject having an autoimmune disease a composition comprising natural killer (NK) cells genetically engineered to express a chimeric antigen receptor (CAR) that binds to CD19, wherein:
(i) the composition comprising the NK cells genetically engineered to express a CAR is administered to the subject in a dosing regimen comprising a dosing cycle, wherein the dosing cycle comprises a first dose, a second dose, and a third dose of the composition;
(ii) each of the first, second, and third doses of the dosing cycle comprises between about 1×10º CAR-expressing NK cells and about 2.5×10º CAR-expressing NK cells;
(iii) the second dose is administered to the subject about 2-4 days after the first dose is administered to the subject, and the third dose is administered to the subject about 2-4 days after the second dose is administered to the subject; and
(iv) about three days prior to administration of the composition comprising NK cells genetically engineered to express a CAR to the subject, the subject is administered a lymphodepleting therapy consisting of a single dose of about 1000 mg/m² of cyclophosphamide.

### EXAMPLES

The following examples are included for illustrative purposes only and are not intended to limit the scope of the present disclosure.

### Example 1: Administration of Anti-CD19 CAR-Expressing NK Cells to Subjects with Systemic Lupus Erythematosus (SLE) and Lupus Nephritis (LN)

### A. First Dosing Regimen

Therapeutic anti-CD19 CAR-expressing NK cell compositions (CD19 CAR NK cells) are administered to subjects with systemic lupus erythematosus (SLE) in accordance with a non-limiting dosing regimen. Subjects having SLE optionally includes subjects with lupus nephritis (LN).

Primary NK cells are isolated by immunoaffinity-based selection from leukapheresis samples from healthy donors and cultured in the presence of a stimulatory cell line. Isolated NK cells are subsequently transduced with a viral vector (e.g., retroviral vector) encoding a non-limiting example of a CD19-directed CAR (see FIG. 1), expanded in culture, and cryopreserved. The CD19-directed CAR contains an extracellular anti-CD19 scFv (e.g., SEQ ID NO: 37), a CD8alpha hinge (e.g., SEQ ID NO: 6) and transmembrane domain (e.g., SEQ ID NO:8), and an intracellular signaling domain containing an OX40 co-stimulatory signaling region (e.g., SEQ ID NO:14) and a CD3zeta signaling domain (e.g., SEQ ID NO:18). The viral vector further contains a sequence encoding a membrane-bound interleukin-15 (mbIL15; e.g., SEQ ID NO:23 or 40), which is separated from the CAR-encoding sequence by a sequence encoding a T2A ribosomal skip sequence (e.g., SEQ ID NO:20, encoded by SEQ ID NO:19). The in vitro and in vivo ability of NK cells expressing the exemplary CD19-directed CAR to exert cytotoxicity against CD19-expressing target cells has been previously demonstrated (see e.g., Morisot et al., J Immuno Ther Canc (2020) 8 (suppl. 3): Abstract 127; PCT Application Nos. PCT/US2020/020824 and PCT/US2023/069403, each of which is incorporated herein by reference in its entirety). The cryopreserved NK cell compositions are thawed prior to intravenous administration to subjects with SLE (e.g., LN).

Subjects are administered a lymphodepleting therapy of 500 mg/m² cyclophosphamide (Cy) and 30 mg/m² fludarabine (Flu) on each of Days −5, −4, and −3. The NK cell composition is administered beginning on Day 0 of the dosing cycle. In particular, subjects are administered a first dose of 1.5×10⁹ CD19 CAR NK cells on Day 0, a second dose of 1.5×10⁹ CD19 CAR NK cells on Day 7, and a third dose of 1.5×10⁹ CD19 CAR NK cells on Day 14. A schematic of the non-limiting dosing regimen is shown in FIG. 2A. Outcome measures are optionally assessed, such as on Day 27 or 28. Subjects who exhibit clinical benefit (e.g., partial renal response (PRR) or complete renal response (CRR)) from treatment with a dosing cycle may be eligible for an additional dosing cycle to deepen or consolidate the response, respectively. Subjects who exhibit initial clinical benefit from treatment with a dosing cycle and subsequently relapse may be eligible for retreatment with an additional dosing cycle. For example, subjects who achieve CRR with a treatment-free interval of at least six months followed by worsening renal function, may be eligible for retreatment.

Outcomes measures may include any of the following: incidence, nature, and severity of treatment-related adverse events; proportion of subjects experiencing dose-limiting toxicities (DLTs); pharmacokinetic parameters, including but not limited to maximum concentration (Cₘₐₓ), time to reach maximum concentration (Tₘₐₓ), area under the concentration time curve (AUC), half-life (t_{1/2}), and duration of persistence of the CD19 CAR NK cells in the peripheral blood and other target tissues such as bone marrow; detection of autoantibodies; detection of complement objective response rate (ORR); disease control rate (DCR); duration of remission (DOR); and overall survival (OS). Outcome measures may also include levels of complement proteins (e.g., C3 and/or C4), activity of complement proteins (e.g., as assessed by CH50), and clinical response (e.g., PRR or CRR). This is a prophetic example.

### B. Second Dosing Regimen

Therapeutic anti-CD19 CAR-expressing NK cell compositions (CD19 CAR NK cells) are administered to subjects with systemic lupus erythematosus (SLE) or lupus nephritis (LN) in accordance with a non-limiting dosing regimen.

Primary NK cells are isolated by immunoaffinity-based selection from leukapheresis samples from healthy donors and cultured in the presence of a stimulatory cell line. Isolated NK cells are subsequently transduced with a viral vector (e.g., retroviral vector) encoding a non-limiting example of a CD19-directed CAR (see FIG. 1), expanded in culture, and cryopreserved. The CD19-directed CAR contains an extracellular anti-CD19 scFv (e.g., SEQ ID NO: 37), a CD8alpha hinge (e.g., SEQ ID NO: 6) and transmembrane domain (e.g., SEQ ID NO:8), and an intracellular signaling domain containing an OX40 co-stimulatory signaling region (e.g., SEQ ID NO:14) and a CD3zeta signaling domain (e.g., SEQ ID NO:18). The viral vector further contains a sequence encoding a membrane-bound interleukin-15 (mbIL15; e.g., SEQ ID NO:40), which is separated from the CAR-encoding sequence by a sequence encoding a T2A ribosomal skip sequence (e.g., SEQ ID NO:20, encoded by SEQ ID NO:19). The cryopreserved NK cell compositions are thawed prior to intravenous administration to subjects with SLE.

Subjects are administered a lymphodepleting therapy of 1000 mg/m² Cy on Day −3, either alone or in combination with 25 mg/m² Flu on each of Days −5, −4, and −3 (FIG. 2B). The NK cell composition is administered beginning on Day 0 of the dosing cycle. In particular, subjects are administered a dose of 3×10⁸ (Cohort A), 1×10⁹ (Cohort B), or 1.5×10⁹ (Cohort C) CD19 CAR NK cells on each of Days 0, 7, and 14. For subjects weighing less than 50 kilograms (kg), subjects are administered a dose of 6×10⁶, 2×10⁷, or 6×10⁷ CD19 CAR NK cells/kg on each of Days 0, 7, and 14. A schematic of the non-limiting dosing regimens is shown in FIG. 2B. Outcome measures are optionally assessed, such as on Day 41 or 42. Subjects who exhibit clinical benefit from treatment with a dosing cycle may be eligible for an additional dosing cycle to deepen or consolidate the response. Subjects who exhibit initial clinical benefit (e.g., partial renal response (PRR) or complete renal response (CRR)) from treatment with a dosing cycle and subsequently relapse may be eligible for retreatment with an additional dosing cycle. For example, subjects who achieve CRR with a treatment-free interval of at least six months followed by worsening renal function, may be eligible for retreatment.

Outcomes measures may include any of the following: incidence, nature, and severity of treatment-related adverse events; proportion of subjects experiencing dose-limiting toxicities (DLTs); pharmacokinetic parameters, including but not limited to maximum concentration (Cₘₐₓ), time to reach maximum concentration (Tₘₐₓ), area under the concentration time curve (AUC), half-life (t_{1/2}), and duration of persistence of the CD19 CAR NK cells in the peripheral blood and other target tissues such as bone marrow; detection of autoantibodies; objective response rate (ORR); disease control rate (DCR); duration of remission (DOR); and overall survival (OS). Outcome measures may also include levels of complement proteins (e.g., C3 and/or C4), activity of complement proteins (e.g., as assessed by CH50), and clinical response (e.g., PRR or CRR). This is a prophetic example.

### C. Third Dosing Regimen

Therapeutic anti-CD19 CAR-expressing NK cell compositions (CD19 CAR NK cells) are administered to subjects with systemic lupus erythematosus (SLE) or lupus nephritis (LN) in accordance with a non-limiting dosing regimen.

Primary NK cells are isolated by immunoaffinity-based selection from leukapheresis samples from healthy donors and cultured in the presence of a stimulatory cell line. Isolated NK cells are subsequently transduced with a viral vector (e.g., retroviral vector) encoding a non-limiting example of a CD19-directed CAR (see FIG. 1), expanded in culture, and cryopreserved. The CD19-directed CAR contains an extracellular anti-CD19 scFv (e.g., SEQ ID NO: 37), a CD8alpha hinge (e.g., SEQ ID NO: 6) and transmembrane domain (e.g., SEQ ID NO:8), and an intracellular signaling domain containing an OX40 co-stimulatory signaling region (e.g., SEQ ID NO:14) and a CD3zeta signaling domain (e.g., SEQ ID NO:18). The viral vector further contains a sequence encoding a membrane-bound interleukin-15 (mbIL15; e.g., SEQ ID NO:40), which is separated from the CAR-encoding sequence by a sequence encoding a T2A ribosomal skip sequence (e.g., SEQ ID NO:20, encoded by SEQ ID NO:19). The cryopreserved NK cell compositions are thawed prior to intravenous administration to subjects with SLE.

Subjects are administered a lymphodepleting therapy of either 500 mg/m² Cy on each of Days −3, −4, and −5 (FIG. 2C) or 1000 mg/m² Cy on Day −3 (FIG. 2D), the Cy alone or in combination with 25 mg/m² fludarabine (Flu) on each of Days −5, −4, and −3. The NK cell composition is administered beginning on Day 0 of the dosing cycle. In particular, subjects are administered a dose of 2×10⁹ CD19 CAR NK cells on each of Days 0, 3, and 7. A schematic of the non-limiting dosing regimens is shown in FIGS. 2C-D. Outcome measures are optionally assessed, such as on Day 41 or 42. Subjects who exhibit clinical benefit from treatment with a dosing cycle may be eligible for an additional dosing cycle to deepen or consolidate the response. Subjects who exhibit initial clinical benefit (e.g., partial renal response (PRR) or complete renal response (CRR)) from treatment with a dosing cycle and subsequently relapse may be eligible for retreatment with an additional dosing cycle. For example, subjects who achieve CRR with a treatment-free interval of at least six months followed by worsening renal function, may be eligible for retreatment.

Outcomes measures may include any of the following: incidence, nature, and severity of treatment-related adverse events; proportion of subjects experiencing dose-limiting toxicities (DLTs); pharmacokinetic parameters, including but not limited to maximum concentration (Cₘₐₓ), time to reach maximum concentration (Tₘₐₓ), area under the concentration time curve (AUC), half-life (t_{1/2}), and duration of persistence of the CD19 CAR NK cells in the peripheral blood and other target tissues such as bone marrow; detection of autoantibodies; objective response rate (ORR); disease control rate (DCR); duration of remission (DOR); and overall survival (OS). Outcome measures may also include levels of complement proteins (e.g., C3 and/or C4), activity of complement proteins (e.g., as assessed by CH50), and clinical response (e.g., PRR or CRR). This is a prophetic example.

### Example 2: Administration of Anti-CD19 CAR-Expressing NK Cells to Subjects with Other Autoimmune Diseases

### A. First Dosing Regimen

Therapeutic anti-CD19 CAR-expressing NK cell compositions (CD19 CAR NK cells) are administered to subjects with myasthenia gravis (MG), idiopathic inflammatory myopathies (IIM; e.g., anti-synthetase syndrome), scleroderma (e.g., systemic sclerosis), or vasculitis (e.g., ANCA vasculitis) in accordance with a non-limiting dosing regimen. The anti-CD19 CAR-expressing NK cell compositions are generated as described in Example 1. Cryopreserved NK cell compositions are thawed prior to intravenous administration to subjects.

Subjects are administered a lymphodepleting therapy of 1000 mg/m² Cy on Day −3, either alone or in combination with 25 mg/m² Flu on each of Days −5, −4, and −3. The NK cell composition is administered beginning on Day 0 of the dosing cycle. In particular, subjects are administered a dose of 3×10⁸ (Cohort A), 1×10⁹ (Cohort B), or 1.5×10⁹ (Cohort C) CD19 CAR NK cells on each of Days 0, 7, and 14. For subjects weighing less than 50 kilograms (kg), subjects are administered a dose of 6×10⁶, 2×10⁷, or 6×10⁷ CD19 CAR NK cells/kg on each of Days 0, 7, and 14. Outcome measures are optionally assessed, such as at the end of the dosing cycle. Subjects who exhibit clinical benefit from treatment with a dosing cycle may be eligible for an additional dosing cycle to deepen or consolidate the response. Subjects who exhibit initial clinical benefit from treatment with a dosing cycle and subsequently relapse and/or exhibit disease progression may be eligible for retreatment with an additional dosing cycle.

Outcomes measures may include any of the following: incidence, nature, and severity of treatment-related adverse events; proportion of subjects experiencing dose-limiting toxicities (DLTs); pharmacokinetic parameters, including but not limited to maximum concentration (Cₘₐₓ), time to reach maximum concentration (Tₘₐₓ), area under the concentration time curve (AUC), half-life (t_{1/2}), duration of persistence of the CD19 CAR NK cells in the peripheral blood and other target tissues such as bone marrow; detection of the presence and/or level of autoantibodies; disease activity (e.g., as measured by a disease activity index), objective response rate (ORR); disease control rate (DCR); duration of remission (DOR); overall survival (OS). This is a prophetic example.

### B. Second Dosing Regimen

Therapeutic anti-CD19 CAR-expressing NK cell compositions (CD19 CAR NK cells) are administered to subjects with myasthenia gravis (MG), idiopathic inflammatory myopathies (IIM; e.g., anti-synthetase syndrome), scleroderma (e.g., systemic sclerosis), or vasculitis (e.g., ANCA vasculitis) in accordance with a non-limiting dosing regimen. CD19-CAR expressing NK cell compositions are generated as described in Example 1. Cryopreserved CD19 CAR NK cells are thawed prior to intravenous administration to subjects.

Subjects are administered a lymphodepleting therapy of either 500 mg/m² Cy on each of Days −3, −4, and −5 or 1000 mg/m² Cy on Day −3, the Cy alone or in combination with 25 mg/m² fludarabine (Flu) on each of Days −5, −4, and −3. The NK cell composition is administered beginning on Day 0 of the dosing cycle. In particular, subjects are administered a dose of 1×10⁹, 1.5×10⁹, or 2×10⁹ CD19 CAR NK cells on each of Days 0, 3, and 7. Outcome measures are optionally assessed, such as at the end of the dosing cycle. Subjects who exhibit clinical benefit from treatment with a dosing cycle may be eligible for an additional dosing cycle to deepen or consolidate the response. Subjects who exhibit initial clinical benefit from treatment with a dosing cycle and subsequently relapse and/or exhibit disease progression may be eligible for retreatment with an additional dosing cycle.

Outcomes measures may include any of the following: incidence, nature, and severity of treatment-related adverse events; proportion of subjects experiencing dose-limiting toxicities (DLTs); pharmacokinetic parameters, including but not limited to maximum concentration (Cₘₐₓ), time to reach maximum concentration (Tₘₐₓ), area under the concentration time curve (AUC), half-life (t_{1/2}), and duration of persistence of the CD19 CAR NK cells in the peripheral blood and other target tissues such as bone marrow; detection of the presence and/or level of autoantibodies; disease activity (e.g., as measured by a disease activity index), objective response rate (ORR); disease control rate (DCR); duration of remission (DOR); overall survival (OS). This is a prophetic example.

### Example 3: In Vitro, Ex Vivo, and In Vivo Cytotoxic Effects of Anti-CD19 CAR-Expressing NK Cells on CD19-Expressing Target Cells

Anti-CD19 CAR-expressing NK cell compositions were generated as described in Example 1, and the cytotoxic activity of the compositions against CD19-expressing target cells was tested in vitro and in vivo.

### A. In Vitro and Ex Vivo Cytotoxicity

The in vitro cytotoxic activity of the anti-CD19 CAR-expressing NK cells and non-transduced (“control”) NK cells was assessed against CD19-positive (CD19+) and CD19-negative (CD19−) subpopulations of peripheral blood mononuclear cells (PBMCs) from healthy donors. Briefly, CD19 CAR NK cells or control NK cells were incubated with PBMCs for four days at effector-to-target (E: T) ratios between 8:1 and 1:8, and the number of CD19+, CD14+, and CD3+ PBMC subpopulations was assessed by flow cytometric analysis. As shown in FIG. 3A, the increased efficacy between control NK cells and CD19 CAR NK cells was only observed in the CD19+ PBMC subpopulation, indicating that the enhanced efficacy of CD19 CAR NK cells is CD19-specific.

The ex vivo cytotoxicity activity of the CD19 CAR NK cells was also assessed against CD19+ B cells in PBMC samples obtained from donors with autoimmune diseases including systemic lupus erythematosus (SLE; n=3), myositis (n=3), scleroderma (n=3), and myasthenia gravis (MG; n=1). CD19 CAR NK or control NK cells were incubated with donor PBMCs for 24 hours at E: T ratios between 4:1 to 1:32. Target PBMCs were collected after 24 hours, and CD19+ B cells (CD45+/CD19+/CD3−/CD56−/CD14−) were isolated from the collected PBMC populations by flow cytometry and quantified. As shown in FIG. 3B, CD19 CAR NK cells exhibited significant cytotoxicity against CD19+ B cells from all donors across multiple E: T ratios.

In a further experiment, control NK cells, CD19 CAR NK cells, or CD19 CAR T cells were incubated with CD19+ B-ALL (REH) or pre-B ALL (Nalm-6) cell lines at various E: T ratios for 24 or 72 hours. Cytotoxicity was assessed via Incucyte® analysis and calculated as the percent of target cell growth inhibition compared to reference wells containing only target cells (mean±SEM; dotted line indicates EC₅₀; n=3 independent donors/E: T ratio). The in vitro cytotoxicity kinetics of the CD19 CAR NK cells were observed to be more rapid than that of the CD19 CAR T cells in both the 24-hour (FIG. 4, top panels) and 72-hour (FIG. 4, bottom panels) analyses. In addition, the cytokine responses of control NK cells, CD19 CAR NK cells, and CD19 CAR T cells were assessed by a Luminex® cytokine assay (mean±SEM; n=3 independent donors). Effector cells were co-cultured with REH or Nalm-6 target cells for 24 hours at a 1:1 ratio. Despite the CD19 CAR NK cells exhibiting more rapid cytotoxicity than CD19 CAR T cells, the cytokine responses of CD19 CAR NK cells were observed to be limited relative to those of the CD19 CAR T cells (FIG. 5).

### B. In Vivo Cytotoxicity

The in vivo cytotoxic activity of the anti-CD19 CAR-expressing NK cells and non-transduced (“control”) NK cells was assessed in a CD19+ xenograft tumor model. In particular, NOD scid gamma (NSG™) mice were injected intravenously (IV) with 2×10⁵ Nalm6 cells on Day −1 and subsequently injected IV with 2×10⁶ or 5×10⁶ CD19 CAR NK cells on each of Days 0, 7, and 14. As controls, NSG mice were injected IV with phosphate buffered saline (PBS) or control NK cells on each of Days 0, 7, and 14. Tumor burden was assessed over approximately 30 days via bioluminescence imaging (BLI; % baseline). As shown in FIG. 6, PBS- and control NK-treated mice reached tumor burden endpoint on Day 22 or Day 25, whereas both of the CD19 CAR NK-treated groups of mice exhibited substantial tumor control (mean±SEM; n=14-17/group).

Together, the data are consistent with a finding that CD19 CAR NK cells exhibit CD19-specific cytotoxicity against CD19-expressing target cells, as well as faster cytotoxic kinetics and limited production of cytokines as compared to CD19 CAR-expressing T cells.

### Example 4: Effects of Anti-CD19 CAR-Expressing NK Cells on CD19-Expressing Cells in Human Subjects

Anti-CD19 CAR-expressing NK cell compositions were generated as described in Example 1 and administered to subjects with relapsed/refractory CD19+ B cell malignancies in accordance with a non-limiting dosing regimen. Prior to administration of the first dose of CD19 CAR NK cells, subjects were administered a lymphodepleting therapy of cyclophosphamide (either 300 mg/m² or 500 mg/m²) and fludarabine (30 mg/m²) on each of Days −5, −4, and −3. A dose of 3×10⁸, 1×10⁹, or 1.5×10⁹ CD19 CAR NK cells was administered on each of Days 0, 7, and 14. In some cases, subjects were eligible to receive additional dosing cycle(s).

The peak concentration (Cₘₐₓ) of the CD19 CAR NK cells and circulating interleukin 15 (IL15) was assessed in subjects. The Cₘₐₓ of IL15, as assessed after lymphodepletion, was not observed to correlate with the Cₘₐₓ of the CD19 CAR NK cells or clinical responses (FIGS. 7A and 7B, respectively). By contrast, the Cₘₐₓ of the CD19 CAR NK cells was observed to be higher among subjects who exhibited a partial response (PR) or complete response (CR) compared to subjects who exhibited stable disease (SD) or progressive disease (PD) (p=0.047; data not shown). These data are consistent with a finding that lymphodepletion-mediated IL15 is not required for achieving sufficient exposure or clinical response to the CD19 CAR NK cells.

The absolute number of CD19+ B cells per microliter (uL) of whole blood was measured in subjects having non-Hodgkin lymphoma (NHL) out to Day 45. CD19+ B cells were significantly reduced in subjects treated with CD19 CAR NK cells over the period of time analyzed (FIG. 7C; each line represents a different subject).

The recovery of B cell numbers and/or subtypes was also assessed in NHL subjects at the end of treatment (EOT; 30 days after last dose of CD19 CAR NK cells); follow-up one (FUP1; 90±14 days after EOT); follow-up two (FUP2; 180±14 days after EOT); follow-up three (FUP3; 270±14 days after EOT); and follow-up four (FUP4; 360±14 days after EOT).

Specifically, the absolute number of B cells per 800 μL of whole blood was measured in subjects through multiple dosing cycles, as well as at multiple follow-up time points. B cells were observed to be substantially depleted in subjects undergoing one or more dosing cycles, with B cell counts recovering to baseline (e.g., Day −5 of Cycle 1; CID-5) levels or beyond by the FUP time points (FIG. 7D).

Different B cell populations were assessed in a representative NHL subject prior to lymphodepletion, as well as at the FUP1, FUP2, FUP3, and FUP4 time points. In particular, B cell receptor (BCR) heavy chains were assessed via mRNA sequencing. Naïve (non-class-switched) B cells were identified by IgM and IgD BCR isotypes, while B cells that have undergone class switching were identified by IgG and IgA BCR isotypes. Prior to lymphodepletion (pre-LD), the subject exhibited a substantial portion of class-switched BCR isotypes, whereas class-switched BCR isotypes were virtually undetectable at FUP1 (FIG. 8A). A relatively small percentage of class-switched BCR isotypes could be detected between FUP2 and FUP4. The same analysis was carried out at FUP1 for an additional four NHL subjects, and similar results were observed across all subjects (FIG. 8B; n=5). In a related experiment, peripheral blood mononuclear cells (PBMCs) from NHL subjects were subjected to transcriptomic analysis via single cell RNA sequencing (10× Genomics; Pleasanton, CA). B cell subpopulations were identified based on differential gene expression specific to each B cell subpopulation. Consistent with the BCR isotype results described above, most subjects exhibited a predominantly naïve B cell population at FUP1, while memory B cells and plasmablasts were virtually undetectable at this time (FIG. 8C; n=5).

The data are consistent with a finding that CD19 CAR NK cell treatment can achieve a substantial reduction in B cell numbers, and that B cells recover after treatment with a predominantly non-class-switched isotype.

### Example 5: Cyclophosphamide-Only Lymphodepleting Therapy in Combination with Anti-CD19 CAR-Expressing NK Cells

Anti-CD19 CAR-expressing NK cell compositions were generated as described in Example 1 and administered to subjects with relapsed/refractory CD19+ B cell malignancies in accordance with an alternative non-limiting dosing regimen. Prior to administration of the first dose of CD19 CAR NK cells, subjects were administered a lymphodepleting therapy of cyclophosphamide (500 mg/m²) and fludarabine (30 mg/m²) on each of Days −5, −4, and −3. A dose of 1.5×10⁹ CD19 CAR NK cells was administered on each of Days 0, 3, and 7. In some cases, subjects were eligible to receive additional dosing cycles.

In addition, subjects with ongoing cytopenias were eligible to receive a dosing cycle preceded by a lymphodepleting therapy of only 500 mg/m² cyclophosphamide (the lymphodepleting therapy did not contain fludarabine). Two such subjects were administered a first cycle of CD19 CAR NK cells preceded by a lymphodepleting therapy of cyclophosphamide (500 mg/m²) and fludarabine (30 mg/m²), and due to ongoing cytopenias, were administered a second cycle of CD19 CAR NK cells preceded by a lymphodepleting therapy of only cyclophosphamide (500 mg/m²). Each cycle consisted of a dose of 1.5×10⁹ CD19 CAR NK cells on each of Days 0, 3, and 7. The concentration of CD19 CAR NK cells was assessed in the peripheral blood of the subjects at various time points during and between the first and second cycles. As shown in FIG. 9 (assessment time points indicated by dots), both subjects achieved similar CD19 CAR NK concentrations in the first and second cycles, despite the second cycle being preceded by cyclophosphamide-only lymphodepleting therapy. These data are consistent with a finding that a cyclophosphamide-only lymphodepleting therapy achieves sufficient exposure of the CD19 CAR NK cells as provided herein.

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

The section headings used herein are for organizational purposes only and are not to be construed as limiting the subject matter described.

### SEQUENCES

| SEQ | | |
|---|---|---|
| ID | | |
| NO | Sequence | Description |
| 1 | GGGGSGGGGSGGGGS | linker |
| 2 | GGGGSGGGGS | linker |
| 3 | GSTSGSGKPGSGEGSTKG | linker |
| 4 | MALPVTALLLPLALLLHAARP | CD8a signal |
| | | sequence |
| 5 | accacgacgccagcgccgcgaccaccaacaccggcgcccaccatcgcgtcgcagcccctgtccct | Human CD8a |
| | gcgcccagaggcgtgccggccagcggcggggggcgcagtgcacacgagggggctggacttcgcc | hinge (nt) |
| | tgtgat | |
| 6 | TTTPAPRPPTPAPTIASQPLSLRPEACRPAAGGAVHTRGLDFACD | Human CD8a |
| | | hinge (aa) |
| 7 | atctacatctgggcgcccttggccgggacttgtggggtccttctcctgtcactggttatcacc | Human CD8a |
| | | transmembrane |
| | | (nt) |
| 8 | IYIWAPLAGTCGVLLLSLVIT | Human CD8a |
| | | transmembrane |
| | | (aa) |
| 9 | actaccacacccgccccgaggccacctacgccggcaccgactatcgccagtcaacccctctctctgc | Human CD8a |
| | gccccgaggcttgccggcctgcggctggtggggcggtccacacccggggcctggattttgcgtgcg | hinge/ |
| | atatatacatctgggcacctcttgccggcacctgcggagtgctgcttctctcactcgttattacgctgtact | transmembrane |
| | gc | (nt) |
| 10 | TTTPAPRPPTPAPTIASQPLSLRPEACRPAAGGAVHTRGLDFACDI | Human CD8a |
| | YIWAPLAGTCGVLLLSLVIT | hinge/ |
| | | transmembrane |
| | | (aa) |
| 11 | FWVLVVVGGVLACYSLLVTVAFIIFWVRS | Human CD28 |
| | | transmembrane |
| | | (aa) |
| 12 | KRSRLLHSDYMNMTPRRPGPTRKHYQPYAPPRDFAAYRS | Human CD28 |
| | | intracellular |
| | | (aa) |
| 13 | cggagggaccagaggctgccccccgatgcccacaagccccctgggggaggcagtttccggacccc | Human OX40 |
| | catccaagaggagcaggccgacgcccactccaccctggccaagatc | (nt) |
| 14 | RRDQRLPPDAHKPPGGGSFRTPIQEEQADAHSTLAKI | Human OX40 |
| | | (aa) |
| 15 | aaacggggcagaaagaaactcctgtatatattcaaacaaccatttatgagaccagtacaaactactcaa | Human 4-1BB |
| | gaggaagatggctgtagctgccgatttccagaagaagaagaaggaggatgtgaactg | (nt) |
| 16 | KRGRKKLLYIFKQPFMRPVQTTQEEDGCSCRFPEEEEGGCEL | Human 4-1BB |
| | | (aa) |
| 17 | agagtgaagttcagcaggagcgcagacgcccccgcgtaccagcagggccagaaccagctctataac | Human |
| | gagctcaatctaggacgaagagaggagtacgatgttttggacaagagacgtggccgggaccctgaga | CD3zeta (nt) |
| | tggggggaaagccgagaaggaagaaccctcaggaaggcctgtacaatgaactgcagaaagataag | |
| | atggcggaggcctacagtgagattgggatgaaaggcgagcgccggaggggcaaggggcacgatg | |
| | gcctttaccagggtctcagtacagccaccaaggacacctacgacgcccttcacatgcaggccctgccc | |
| | cctcgc | |
| 18 | RVKFSRSADAPAYQQGQNQLYNELNLGRREEYDVLDKRRGRDP | Human |
| | EMGGKPRRKNPQEGLYNELQKDKMAEAYSEIGMKGERRRGKG | CD3zeta (aa) |
| | HDGLYQGLSTATKDTYDALHMQALPPR | |
| 19 | ggctctggcgagggaaggggttccctgcttacttgcggcgacgtcgaagagaatcccggtccg | Human T2A |
| | | (nt) |
| 20 | GSGEGRGSLLTCGDVEENPGP | Human T2A |
| | | (aa) |
| 21 | aactgggtcaacgtgattagcgatttgaagaaaatcgaggaccttatacagtctatgcatattgacgctac | Human IL-15 |
| | actgtatactgagagtgatgtacacccgtcctgtaaggtaacggccatgaaatgctttcttctggagctcc | (nt) |
| | aggtcatcagcttggagtctggggacgcaagcatccacgatacggttgaaaacctcatcatccttgcga | |
| | acaactctctctcatctaatggaaacgttacagagagtgggtgtaaggagtgcgaagagttggaagaaa | |
| | aaaacatcaaagaatttcttcaatccttcgttcacatagtgcaaatgttcattaacacgtcc | |
| 22 | NWVNVISDLKKIEDLIQSMHIDATLYTESDVHPSCKVTAMKCFLL | Human IL-15 |
| | ELQVISLESGDASIHDTVENLIILANNSLSSNGNVTESGCKECEEL | (aa) |
| | EEKNIKEFLQSFVHIVQMFINTS | |
| 23 | NWVNVISDLKKIEDLIQSMHIDATLYTESDVHPSCKVTAMKCFLL | mbIL15 (aa) |
| | ELQVISLESGDASIHDTVENLIILANNSLSSNGNVTESGCKECEEL | |
| | EEKNIKEFLQSFVHIVQMFINTSTTTPAPRPPTPAPTIASQPLSLRPE | |
| | ACRPAAGGAVHTRGLDFACDIYIWAPLAGTCGVLLLSLVITLYC | |
| 24 | GVSLPDYG | CD19 CDR-H1 |
| 25 | IWGSETT | CD19 CDR-H2 |
| 26 | AKHYYYGGSYAMDY | CD19 CDR-H3 |
| 27 | QDISKY | CD19 CDR-L1 |
| 28 | HT | CD19 CDR-L2 |
| 29 | QQGNTLPYT | CD19 CDR-L3 |
| 30 | DYGVS | CD19 CDR-H1 |
| 31 | VIWGSETTYYNSALKS | CD19 CDR-H2 |
| 32 | HYYYGGSYAMDY | CD19 CDR-H3 |
| 33 | RASQDISKYLN | CD19 CDR-L1 |
| 34 | HTSRLHS | CD19 CDR-L2 |
| 35 | QVQLQESGPGLVKPSQTLSLTCTVSGVSLPDYGVSWIRQPPGKGL | CD19 VH |
| | EWIGVIWGSETTYYNSALKSRLTISKDNSKNQVSLKLSSVTAADT | |
| | AVYYCAKHYYYGGSYAMDYWGQGTSVTVSS | |
| 36 | DIQMTQSPSSLSASVGDRVTITCRASQDISKYLNWYQQKPGGTV | CD19 VL |
| | KLLIYHTSRLHSGVPSRFSGSGSGTDFTLTISSLQPEDIATYFCQQG | |
| | NTLPYTFGGGTKLEIT | |
| 37 | DIQMTQSPSSLSASVGDRVTITCRASQDISKYLNWYQQKPGGTV | CD19 scFv |
| | KLLIYHTSRLHSGVPSRFSGSGSGTDFTLTISSLQPEDIATYFCQQG | |
| | NTLPYTFGGGTKLEITGGGGSGGGGSGGGGSQVQLQESGPGLVK | |
| | PSQTLSLTCTVSGVSLPDYGVSWIRQPPGKGLEWIGVIWGSETTY | |
| | YNSALKSRLTISKDNSKNQVSLKLSSVTAADTAVYYCAKHYYY | |
| | GGSYAMDYWGQGTSVTVSS | |
| 38 | DIQMTQSPSSLSASVGDRVTITCRASQDISKYLNWYQQKPGGTV | CD19 CAR |
| | KLLIYHTSRLHSGVPSRFSGSGSGTDFTLTISSLQPEDIATYFCQQG | |
| | NTLPYTFGGGTKLEITGGGGSGGGGSGGGGSQVQLQESGPGLVK | |
| | PSQTLSLTCTVSGVSLPDYGVSWIRQPPGKGLEWIGVIWGSETTY | |
| | YNSALKSRLTISKDNSKNQVSLKLSSVTAADTAVYYCAKHYYY | |
| | GGSYAMDYWGQGTSVTVSSTTTPAPRPPTPAPTIASQPLSLRPEA | |
| | CRPAAGGAVHTRGLDFACDIYIWAPLAGTCGVLLLSLVITLYCR | |
| | RDQRLPPDAHKPPGGGSFRTPIQEEQADAHSTLAKIRVKFSRSAD | |
| | APAYQQGQNQLYNELNLGRREEYDVLDKRRGRDPEMGGKPRR | |
| | KNPQEGLYNELQKDKMAEAYSEIGMKGERRRGKGHDGLYQGL | |
| | STATKDTYDALHMQALPPR | |
| 39 | PPPRLLFFLLFLTPMEVRPEEPLVVKVEEGDNAVLQCLKGTSDGP | human CD19 |
| | TQQLTWSRESPLKPFLKLSLGLPGLGIHMRPLAIWLFIFNVSQQM | (UniProt No. |
| | GGFYLCQPGPPSEKAWQPGWTVNVEGSGELFRWNVSDLGGLGC | P15391) |
| | GLKNRSSEGPSSPSGKLMSPKLYVWAKDRPEIWEGEPPCLPPRDS | |
| | LNQSLSQDLTMAPGSTLWLSCGVPPDSVSRGPLSWTHVHPKGPK | |
| | SLLSLELKDDRPARDMWVMETGLLLPRATAQDAGKYYCHRGN | |
| | LTMSFHLEITARPVLWHWLLRTGGWKVSAVTLAYLIFCLCSLVG | |
| | ILHLQRALVLRRKRKRMTDPTRRFFKVTPPPGSGPQNQYGNVLS | |
| | LPTPTSGLGRAQRWAAGLGGTAPSYGNPSSDVQADGALGSRSPP | |
| | GVGPEEEEGEGYEEPDSEEDSEFYENDSNLGQDQLSQDGSGYEN | |
| | PEDEPLGPEDEDSFSNAESYENEDEELTQPVARTMDFLSPHGSAW | |
| | DPSREATSLGSQSYEDMRGILYAAPQLRSIRGQPGPNHEEDADSY | |
| | ENMDNPDGPDPAWGGGGRMGTWSTR | |
| 40 | NWVNVISDLKKIEDLIQSMHIDATLYTESDVHPSCKVTAMKCFLL | mbIL15 (aa) |
| | ELQVISLESGDASIHDTVENLIILANNSLSSNGNVTESGCKECEEL | |
| | EEKNIKEFLQSFVHIVQMFINTSTTTPAPRPPTPAPTIASQPLSLRPE | |
| | ACRPAAGGAVHTRGLDFACDIYIWAPLAGTCGVLLLSLVIT | |
| 41 | TTTPAPRPPTPAPTIASQPLSLRPEACRPAAGGAVHTRGLDFACDI | Human CD8a |
| | YIWAPLAGTCGVLLLSLVITLYC | hinge/ |
| | | transmembrane |
| | | (aa) |

1-78. (canceled)

79. A method of treating a subject having an autoimmune disease, the method comprising administering to a subject having an autoimmune disease a composition comprising natural killer (NK) cells genetically engineered to express a chimeric antigen receptor (CAR) that binds to CD19, wherein the genetically engineered NK cells are allogeneic to the subject.

80. The method of claim 79, wherein the autoimmune disease comprises systemic lupus erythematosus (SLE), lupus nephritis (LN), systemic sclerosis (SSc), myositis, vasculitis, multiple sclerosis (MS), or myasthenia gravis (MG).

81. The method of claim 79, wherein the autoimmune disease comprises systemic lupus erythematosus (SLE).

82. The method of claim 79, wherein the composition comprising the genetically engineered NK cells is administered to the subject in a dosing regimen comprising a dosing cycle comprising a first dose, a second dose, and a third dose of the composition comprising the genetically engineered NK cells.

83. The method of claim 82, wherein the second dose is administered to the subject between about 5 days after and about 10 days after the first dose is administered to the subject, and the third dose is administered to the subject between about 5 days after and about 10 days after the second dose is administered to the subject.

84. The method of claim 82, wherein the second dose is administered to the subject between about 2 days after and about 4 days after the first dose is administered to the subject, and the third dose is administered to the subject between about 2 days after and about 4 days after the second dose is administered to the subject.

85. The method of claim 82, wherein each dose of the dosing cycle comprises between about 3×10⁸ CAR-expressing NK cells and about 3×10⁹ CAR-expressing NK cells, each inclusive.

86. The method of claim 79, wherein:
(i) the method comprises administering a lymphodepleting therapy to the subject prior to administration of the composition comprising the genetically engineered NK cells; or
(ii) prior to administration of the composition comprising the genetically engineered NK cells to the subject, the subject has been administered a lymphodepleting therapy.

87. The method of claim 86, wherein the lymphodepleting therapy comprises administration of cyclophosphamide and does not comprise administration of fludarabine.

88. The method of claim 86, wherein the lymphodepleting therapy consists of cyclophosphamide.

89. The method of claim 79, wherein the subject has relapsed following treatment with and/or is refractory to a prior line of therapy for the autoimmune disease.

90. A method of preparing a subject having an autoimmune disease for treatment with a composition comprising natural killer (NK) cells genetically engineered to express a chimeric antigen receptor (CAR) that binds to CD19, the method comprising administering a lymphodepleting therapy to a subject prior to administration of the composition comprising the genetically engineered NK cells to the subject, wherein the lymphodepleting therapy consists of cyclophosphamide.

91. The method of claim 90, wherein the autoimmune disease comprises systemic lupus erythematosus (SLE), lupus nephritis (LN), systemic sclerosis (SSc), myositis, vasculitis, multiple sclerosis (MS), or myasthenia gravis (MG).

92. The method of claim 90, wherein the autoimmune disease comprises systemic lupus erythematosus (SLE).

93. The method of claim 90, wherein the lymphodepleting therapy comprises a single dose of cyclophosphamide at between about 500 mg/m² and about 1500 mg/m².

94. The method of claim 93, wherein the single dose of cyclophosphamide is administered to the subject about 3 days before administration of the composition comprising the genetically engineered NK cells.

95. The method of claim 90, wherein the genetically engineered NK cells are allogeneic to the subject.

96. A method of reducing B cells in a subject having an autoimmune disease, the method comprising administering to a subject having an autoimmune disease a composition comprising natural killer (NK) cells genetically engineered to express a chimeric antigen receptor (CAR) that binds to CD19, wherein the genetically engineered NK cells are allogeneic to the subject.

97. The method of claim 96, wherein, among a plurality of subjects treated according to the method, the number of peripheral B cells in the subjects is significantly reduced within about 10 days, within about 15 days, or within about 30 days after administration of a first dose of the composition comprising the genetically engineered NK cells to the subject.

98. The method of claim 96, wherein, among a plurality of subjects treated according to the method, the number of peripheral B cells in the subjects is significantly reduced for at least about 15 days, at least about 1 month, at least about 2 months, at least about 3 months, at least about 6 months, or at least about 9 months following administration of a final dose of the composition comprising the genetically engineered NK cells to the subject.

99. A method of reducing B cells in a subject having an autoimmune disease, the method comprising administering to a subject having an autoimmune disease a composition comprising natural killer (NK) cells genetically engineered to express a chimeric antigen receptor (CAR) that binds to CD19, wherein:
(i) the method reduces peripheral B cells in the subject by at least about 90%;
(ii) the composition comprising the genetically engineered NK cells is administered to the subject in a dosing regimen comprising a dosing cycle, and peripheral B cells are significantly reduced in the subject for the duration of the dosing cycle; or
(iii) at least about 75% of repopulating peripheral B cells are non-class-switched B cells.

100. The method of claim 99, wherein, among a plurality of subjects treated according to the method, the number of peripheral B cells in the subjects is significantly reduced within about 10 days, within about 15 days, or within about 30 days after administration of a first dose of the composition comprising the genetically engineered NK cells to the subject.

101. The method of claim 99, wherein, among a plurality of subjects treated according to the method, the number of peripheral B cells in the subjects is significantly reduced for at least about 15 days, at least about 1 month, at least about 2 months, at least about 3 months, at least about 6 months, or at least about 9 months following administration of a final dose of the composition comprising the genetically engineered NK cells to the subject.

102. The method of claim 99, wherein the genetically engineered NK cells are allogeneic to the subject.

103. A method of reducing the level of an autoantibody in a subject having an autoimmune disease, the methods comprising administering to a subject having an autoimmune disease a composition comprising natural killer (NK) cells genetically engineered to express a chimeric antigen receptor (CAR) that binds to CD19.

104. The method of claim 103, wherein, among a plurality of subjects treated according to the method, the level of an autoantibody in the subject is reduced by an average of at least about 50%, at least about 60%, at least about 70%, at least about 80%, at least about 90%, or at least about 95%.

105. The method of claim 103, wherein the autoimmune disease comprises systemic lupus erythematosus (SLE), lupus nephritis (LN), systemic sclerosis (SSc), myositis, vasculitis, multiple sclerosis (MS), or myasthenia gravis (MG).

106. A kit comprising (i) a composition comprising natural killer (NK) cells genetically engineered to express a chimeric antigen receptor (CAR) that binds to CD19; and (ii) instructions for administering the composition to a subject having an autoimmune disease.

107. The kit of claim 106, wherein the autoimmune disease comprises systemic lupus erythematosus (SLE), lupus nephritis (LN), systemic sclerosis (SSc), myositis, vasculitis, multiple sclerosis (MS), or myasthenia gravis (MG).

108. The kit of claim 106, wherein the genetically engineered NK cells are allogeneic to the subject.


SEQUENCE LISTING (41 sequences)

SEQ ID NO: 1 - protein, 15 aa, synthetic construct
GGGGSGGGGSGGGGS

SEQ ID NO: 2 - protein, 10 aa, synthetic construct
GGGGSGGGGS

SEQ ID NO: 3 - protein, 18 aa, synthetic construct
GSTSGSGKPGSGEGSTKG

SEQ ID NO: 4 - protein, 21 aa, synthetic construct
MALPVTALLLPLALLLHAARP

SEQ ID NO: 5 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 6 - protein, 45 aa, synthetic construct
TTTPAPRPPTPAPTIASQPLSLRPEACRPAAGGAVHTRGLDFACD

SEQ ID NO: 7 - DNA, 63 bp, synthetic construct
  source 1..63: mol_type = other DNA
atctacatctgggcgcccttggccgggacttgtggggtccttctcctgtcactggttatcacc

SEQ ID NO: 8 - protein, 21 aa, synthetic construct
IYIWAPLAGTCGVLLLSLVIT

SEQ ID NO: 9 - DNA, 207 bp, synthetic construct
  source 1..207: mol_type = other DNA
actaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 10 - protein, 66 aa, synthetic construct
TTTPAPRPPTPAPTIASQPLSLRPEACRPAAGGAVHTRGLDFACDIYIWAPLAGTCGVLLLSLVIT

SEQ ID NO: 11 - protein, 29 aa, synthetic construct
FWVLVVVGGVLACYSLLVTVAFIIFWVRS

SEQ ID NO: 12 - protein, 39 aa, synthetic construct
KRSRLLHSDYMNMTPRRPGPTRKHYQPYAPPRDFAAYRS

SEQ ID NO: 13 - DNA, 111 bp, synthetic construct
  source 1..111: mol_type = other DNA
cggagggaccagaggctgccccccgatgcccacaagccccctgggggaggcagtttccggacccccatccaagaggagcaggccgacgcccactccaccctggccaagatc

SEQ ID NO: 14 - protein, 37 aa, synthetic construct
RRDQRLPPDAHKPPGGGSFRTPIQEEQADAHSTLAKI

SEQ ID NO: 15 - DNA, 126 bp, unidentified
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 16 - protein, 42 aa, synthetic construct
KRGRKKLLYIFKQPFMRPVQTTQEEDGCSCRFPEEEEGGCEL

SEQ ID NO: 17 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 18 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 63 bp, unidentified
  source 1..63: mol_type = other DNA
ggctctggcgagggaaggggttccctgcttacttgcggcgacgtcgaagagaatcccggtccg

SEQ ID NO: 20 - protein, 21 aa, synthetic construct
GSGEGRGSLLTCGDVEENPGP

SEQ ID NO: 21 - DNA, 342 bp, synthetic construct
  source 1..342: mol_type = other DNA
aactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtcc

SEQ ID NO: 22 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 23 - protein, 183 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 24 - protein, 8 aa, synthetic construct
GVSLPDYG

SEQ ID NO: 25 - protein, 7 aa, synthetic construct
IWGSETT

SEQ ID NO: 26 - protein, 14 aa, synthetic construct
AKHYYYGGSYAMDY

SEQ ID NO: 27 - protein, 6 aa, synthetic construct
QDISKY

SEQ ID NO: 28 - skipped

SEQ ID NO: 29 - protein, 9 aa, synthetic construct
QQGNTLPYT

SEQ ID NO: 30 - protein, 5 aa, synthetic construct
DYGVS

SEQ ID NO: 31 - protein, 16 aa, synthetic construct
VIWGSETTYYNSALKS

SEQ ID NO: 32 - protein, 12 aa, synthetic construct
HYYYGGSYAMDY

SEQ ID NO: 33 - protein, 11 aa, synthetic construct
RASQDISKYLN

SEQ ID NO: 34 - protein, 7 aa, synthetic construct
HTSRLHS

SEQ ID NO: 35 - protein, 120 aa, synthetic construct
QVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSS

SEQ ID NO: 36 - protein, 107 aa, synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEIT

SEQ ID NO: 37 - protein, 242 aa, synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSS

SEQ ID NO: 38 - protein, 460 aa, synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 555 aa, Homo sapiens
PPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLGSQSYEDMRGILYAAPQLRSIRGQPGPNHEEDADSYENMDNPDGPDPAWGGGGRMGTWSTR

SEQ ID NO: 40 - protein, 180 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVIT

SEQ ID NO: 41 - protein, 69 aa, synthetic construct
TTTPAPRPPTPAPTIASQPLSLRPEACRPAAGGAVHTRGLDFACDIYIWAPLAGTCGVLLLSLVITLYC